SEQUENCE LISTING (66 sequences)

SEQ ID NO: 1 - DNA, 500 bp, Saccharomyces cerevisiae
  source 1..500: mol_type = other DNA
tatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacaca

SEQ ID NO: 2 - DNA, 500 bp, Saccharomyces cerevisiae
  source 1..500: mol_type = other DNA
ataggaaaaaaccgagcttcctttcatccggcgcggctgtgttctacatatcactgaagctccgggtattttaagttatacaagggaaagatgccggctagactagcaagttttaggctgcttaacattatggataggcggataaagggcccaaacaggattgtaaagcttagacgcttctggttggacaatggtacgtttgtgtattaagtaaggcttggctggggatagcaacattgggcagagtatagaagaccacaaaaaaaaggtatataagggcagagaagtctttgtaatgtgtgtaacttctcttccatgtgtaatcagtatttctacttacttcttaaatatacagaagtaagacagataaccaacagcctttcccagatatacatatatatctttatttcagcttaaacaataattatatttgtttaactcaaaaataaaaaaaaaaaaccaaactcacgcaactaattattccataataaaataacaac

SEQ ID NO: 3 - DNA, 500 bp, Saccharomyces cerevisiae
  source 1..500: mol_type = other DNA
ccattgggccgatgaagttagtcgacggatagaagcggttgtcccctttcccggcgagccggcagtcgggccgaggttcggataaattttgtattgtgttttgattctgtcatgagtattacttatgttctctttaggtaaccccaggttaatcaatcacagtttcataccggctagtattcaaattatgacttttcttctgcagtgtcagccttacgacgattatctatgagctttgaatatagtttgccgtgattcgtatctttaattggataataaaatgcgaaggatcgatgacccttattattatttttctacactggctaccgatttaactcatcttcttgaaagtatataagtaacagtaaaatataccgtacttctgctaatgttatttgtcccttatttttcttttcttgtcttatgctatagtacctaagaataacgactattgttttgaactaaacaaagtagtaaaagcacataaaagaattaagaaa

SEQ ID NO: 4 - DNA, 500 bp, Saccharomyces cerevisiae
  source 1..500: mol_type = other DNA
atttattgaaaagtaaatatctcgtaacccggatgctttgggcggtcgggttttgctactcgtcatccgatgagaaaaactgttcccttttgccccaggtttccattcatccgagcgatcacttatctgacttcgtcactttttcatttcatccgaaacaatcaaaactgaagccaatcaccacaaaattaacactcaacgtcatctttcactaccctttacagaagaaaatatccatagtccggactagcatcccagtatgtgactcaatattggtgcaaaagagaaaagcataagtcagtccaaagtccgcccttaaccaggcacatcggaattcacaaaacgtttctttattatataaaggagctgcttcactggcaaaattcttattatttgtcttggcttgctaatttcatcttatcctttttttcttttcacacccaaatacctaacaattgagagaaaactcttagcataacataacaaaaagtcaacgaaaa

SEQ ID NO: 5 - DNA, 500 bp, Saccharomyces cerevisiae
  source 1..500: mol_type = other DNA
caaaaaaacaatggaagaacaaagaaaatttagcggaagtaaaaataacagccgaaagccaaattcaggcttatcttgcctactctttcttttatcgaattcctttaggccgttgcaatagaaaagtaataaaaacgcatatacgtaagttgtagtcagtgtaattgcaatctattatgcgcatcaggtgcgcatactacatccattggtgcacaaaaaaaggaacgcagacaagaaaattattcagtttgctgttcgtgatgagccatccctgaatatgactaatgttaatgttcaatttgggatcttatttttttttgtgcagtaataagaatctttgaaaaaaaactatataagcctatatagtttgtaagatataagacaaaacacacctgcttttccactacacattttcgttattatataaaaaagacagccaagtatacttgtcaacaaaataaactcatagcaattacactataaaaacaatagcatcaaaa

SEQ ID NO: 6 - DNA, 500 bp, Saccharomyces cerevisiae
  source 1..500: mol_type = other DNA
tggcaatcccctccgatcgtccgcggcaaaatggtcgtcaatcggacaaagggggatgatgggatctggtaatagaagaaaatatggactaaaggtagccgctaaagcgatccaggcatgtgttgccaatgatgtaagtcaagcgaaggaaatggttcagtaatatgatagacagactgcacttcaagggtgcgccccctcccccgcgcatatgcttacaacgcaaaataattgacgtttaatgtggatacttatcgtaatcgctgcattatagatttcgagtcatgttcacttaaccccacatatttatatagaacgcatcttcaaagtacttataaagtttagttttacatttttctgctttctatttcttctttttcggttcttcttcatgccagttggcatggcttaagagctttacttgtcgcttttatttaaaaccttctctcgggagaagacaattgttgatatacagtaattgtatttgcattatcactgct

SEQ ID NO: 7 - DNA, 344 bp, Saccharomyces cerevisiae
  source 1..344: mol_type = other DNA
aacgtctatctatttatttttataactccgggatgtcattgccggtggtccgaaaatcggcaaataaggaaataagggaagaatatgcagtagtcaaatcatcagtgttctctttgatacctttcagggctaggaatagtgggggtggagtataatatcaaaaaccggacttaacattattggttcggttggaattggctataggcaaactagtctccggcatgatatataaatgacagcctgcaattgtatgttactacactcttgacttgtcgactacagtcgctgctcaggcacgagaataggaggtaagaaggtaacgtacgtatatatataaaatcgta

SEQ ID NO: 8 - DNA, 500 bp, Saccharomyces cerevisiae
  source 1..500: mol_type = other DNA
gagctccgtggaataggcgagcggctgagtggttctccaagctacggtttttacgtgtagccccatgtgagcaagccaaacaagggcccttaaaggcgtgactacaaaaaggggcgggttggaaggtcatctgcagcgagatacgaaaagattttttgccagatttgcggttgggcggctatttcggtattgttggggtaacaaacgttggggaagactgcattttcttacagctttttttcgttatcgcgggttgggcggctatggcgccttctcctctgtactccaacctgtcagagacaccaagctgtatataaagcaccttggttggatcgtatttccctgagatcttgctataggttcattttatatatcgtccaatagcaataacaatacaacagaaactactagcatctgtttataagaaaaaggcaaatagtcgacagctaacacagatataactaaacaaccacaaaacaactcatatacaaacaaataat

SEQ ID NO: 9 - DNA, 500 bp, Saccharomyces cerevisiae
  source 1..500: mol_type = other DNA
ccctatctttttttttttctcgcaatctggggaaagcttttctcatgcttatacgtgatttgttatataagggattgctatttcaggcatcattcacctccttttgtatccttagtttcactgcatttgatatatatatatacgtatctgtagtttccttccattacataacgcataatatactatttccatagtctatcttacatcttttttcttacttttgttaaggaacggataacgataaaacaaaaagagagatttaagattacttctgtaacttttttgatccattaccaaaactatattttttttcttttctctcctctggcattaaacacagttattgctacagctaatcatcgatataataatacatcacattaactgtctataagaggctggtacttagtagatggtgagaattttttatttttgtattttaacttcatttttgtaaacaagttggaactggaacttactatagaacaagagcttaaacc

SEQ ID NO: 10 - DNA, 499 bp, Saccharomyces cerevisiae
  source 1..499: mol_type = other DNA
gttgtatcctattggatcacgggcgacggacaagacccgaagtgcggaccggcatggtcagcttgcacggaagctttaagggtttcccttgtttcggcattagaagaggcatttcgcacgttttaccgggtcagaaacttcgaggaagctgtgacaattggaaaaaaaggcaaaactaaatgcaatgtatccggttgcccatgcattatttgtgatgttttcggatgtagttcgctgcgctccgcggcgatatatcctctagcgagaggcatatgtataaatatatatatatatatctaacaaaagcattcaagtttctttctctggtgttacgtctttgttcgactttctctgcttacagccctgtatgaccaaagaaaaaataaaaagacagctacataccagcagaaattttttatagtattacactatacatccaagttttttcacaattatttattgtttttctcacatagaaaattccgcatactgcgattat

SEQ ID NO: 11 - DNA, 500 bp, Saccharomyces cerevisiae
  source 1..500: mol_type = other DNA
gaaagcttattactgagttttgcggagcatcgctcggagcggcggaattgaatcgaaccgccgtgctattaccgaacaaaaaaattcgaaagcataaactcagtagtgaaaaacttgagaattttcagatgagtggcgactttccagtccttgcggttttgtcaccttagtcagctagtaaggaggccgtgtgggttagagtggctacaatcctcaaagggcacttctagaacccacggtgaattttttttggcatgataaatcggtagaatcggtgaagtaattacccaaaaaaggatcgggattgtgtttctcgtaattccgtattattgccgatggcatcgactacttcttttttcagaaaccccaacaagggtctattgtaattgtatataaacctttttgtaatggatatatacatgtggtactatttctcctcatcctgctccatcgaaaatcctcatacgaagagttaggaaagcaaagaaaacaacaaaaac

SEQ ID NO: 12 - DNA, 412 bp, Saccharomyces cerevisiae
  source 1..412: mol_type = other DNA
agacacaatgcgaaaaatcgcgcagggacataatttttgttttcattattctttcgcttattccctccgttagctccaccgcttttttgattggaatttcctttcggcaatggctttccggttaccacgcctcgggtttcgcatcccgaaaagcatatctacacaagaaaaatgaatgataaacaattgatgagtggcgctatttcccttatcatctcattattgtacttagtatcgtctattatcaggagaaatcgcatgaactaagcccattttctcacccttctgccttcttatataaagcttgctgggaaccgaacacaaactccacaagtccgtagcagctcttctcttttgtcttttatatatcataaacatcgctacatagtaataacactaacgcacgctagaa

SEQ ID NO: 13 - DNA, 500 bp, Saccharomyces cerevisiae
  source 1..500: mol_type = other DNA
aggggtagcggctttttcatcaactcgattattaccctttagagaccttccctaaagtgagcggcaattatttccggatgttagtagggtaatatggttacggatttgtgacacaaaagggcttttcaacagtcggtctgggttgaaggattttcaggatgacgaagctttcaataagagggactggactgttaacgcggggaattataggttactttccttgatctggctctggctctggctctgattttggctcttgtactcctcggacttcttgacttgtaacgaaatacgtcttttgtccttctcttcttcttccatagtaggggcgaatgaggggagcatagtggatccttctaaccatctagaatggggtggacaacatataaaagaagagcaatcttgcagcgcagtcatatttatgctaagtatatcattatttcttgctagcgtaagtcataaaaaataggaaataatcacatatatacaagaaattaaat

SEQ ID NO: 14 - DNA, 500 bp, Saccharomyces cerevisiae
  source 1..500: mol_type = other DNA
agcctagtcccggtaaaccgcaaacggaccttaattgtgacgaagggcccaaatttgatgggtcggtgttaatgattagtcctcattgtcataataaagtgtgatgatggaggcaatgatgatatacggtagtactactgctcgaggtgctatcttttaaccaatcctttgagattcttgtcgccacggagttactaccttttacaaaccgtaatgtcacattttgcatatatcttatgtataaatatatagttcacttactacttgttctcgttttgttaactttcttgttgtagttcttcttgttcttggcgtttccccctttgttttctatctgcttcataagtaaagtgcaaagcattttggaagatattatcaattgagtcattgaaagaaacttggcatcttccctattactaaaactaagaatacttgattcaagaaagaagtttatattagttttagccgtaagataacataacaaagaagaagaaagaaaa

SEQ ID NO: 15 - DNA, 500 bp, Saccharomyces cerevisiae
  source 1..500: mol_type = other DNA
tcgatcagctccaattaaatgaagactattcgccgtaccgttcccagatgggtgcgaaagtcagtgatcgaggaagttattgagcgcgcggcttgaaactatttctccatctcagagccgccaagcctaccattattctccaccaggaagttagtttgtaagcttctgcacaccatccggacgtccataattcttcacttaacggtcttttgcccccccttctactataatgcattagaacgttacctggtcatttggatggagatctaagtaacacttactatctcctatggtactatcctttaccaaaaaaaaaaaaaaaaaaaaaaaaaaaaaaatcagcaaagtgaagtaccctcttgatgtataaatacattgcacatcattgttgagaaatagttttggaagttgtctagtccttctcccttagatctaaaaggaagaagagtaacagtttcaaaagtttttcctcaaagagattaaatactgctactgaaaat

SEQ ID NO: 16 - DNA, 500 bp, Saccharomyces cerevisiae
  source 1..500: mol_type = other DNA
acatagtactgtacgattactgtacgattaatctatccacttcagatgttcaacaattccttttggcattacgtattaatacttcataggatcggcaccctcccttaagcctcccctaaatgctttcggtacccctttaagacaactatctcttaaccttctgtatttacttgcatgttacgttgagtctcattggaggtttgcatcatatgtttaggtttttttggaaacgtggacggctcatagtgattggtaaatgggagttacgaataaacgtatcttaaagggagcggtatgtaaaatggatagatgatcatgaatacagtacgaggtgtaaagaatgatgggactgagagggcaattatcatccctcagaatcaacatcacaaacatatataaagctcccaattctgccccaaagttttgtccctaggcatttttaatctttgtatctgtgctctttactttagtagaaaggtatataaaaaagtatagtcaag

SEQ ID NO: 17 - DNA, 488 bp, Saccharomyces cerevisiae
  source 1..488: mol_type = other DNA
aagttcttgactacccctatctcacactagtacgtaattcaatgtatcattcgtattgtaagtagatagagacgcaatacaggaaagctgaccttccttccaatcaccacggctgaaatgctttgttgaccaattacggacgcttaagagcggacgcggctggaacggctccatcctaaatcggcggagggagaactccgataccagccgacatggcaataatagtgacagtagatgctaccagccccgcaataatttcacagtagatcatcaacagtctcctcatttctggaaatgatcagcaacttcgacggatttaactctcaagcagttacgcactccgagaacagccgtgatcatctttgaacaagcaaaatatataaagcaggagaactgtcctacctagagctagaatagccataactaactatgtaacattctacagatcaatcaaaaacaatcttcaatcacagaaaaaaataaaaggc

SEQ ID NO: 18 - DNA, 329 bp, Saccharomyces cerevisiae
  source 1..329: mol_type = other DNA
ttttctagttcttcttctgcaatattgccttttgggaagaaggatcgaaagtagccatttgcagacacgtttttactatatttactgtatcttcgattgcgcggctaaagttgccatattattattatattgcagctcaaccccgcatttccggagttttctttttttttatttggggtaatttggaggtcggcggctattggtgggccggaaatggtgacacacttgtaatatataaggaggaaatcctacatgtgtataagcgaaatcacaaggataataatgtattgctaaacaccctcaagaaagaaaataatcataacgaaatc

SEQ ID NO: 19 - DNA, 500 bp, Saccharomyces cerevisiae
  source 1..500: mol_type = other DNA
cggatggaatcgccgcttttgaattcacctccggggtattattattattcttagtagtcgcggtcgtgcggacacccggagttatgcgggcccgaaagctcattatgtagtaaagctaggtaatgttaagggcgtaagagccaacgcaaggcagcaatagcctggtattcccacatatcaagaaagcttaaaaagttgagacagggaatttgaaggcgaagattgccgaactggccaatacccactactttttttttggtttgcttggtttcttcctgtcgcttgccaacttgtggcatcttccccacactatattataaggatcgtcctatgtataggcaatattatccatttcactcgctaacaaatgtacgtatatatatggagcaacaagtagtgcaattacagacgtgtattttgtcttgatcttgctttttgtatgataggcctaagaataacagtgcgaacatataagaaacatccctcatactaccacacat

SEQ ID NO: 20 - DNA, 442 bp, Saccharomyces cerevisiae
  source 1..442: mol_type = other DNA
agaccttttttttctttttctgctttttcgtcatccccacgttgtgccattaatttgttagtgggcccttaaatgtcgaaatattgctaaaaattggcccgagtcattgaaaggctttaagaatataccgtacaaaggagtttatgtaatcttaataaattgcatatgacaatgcagcacgtgggagacaaatagtaataatactaatctatcaatactagatgtcacagccactttggatccttctattatgtaaatcattagattaactcagtcaatagcagattttttttacaatgtctactgggtggacatctccaaacaattcatgtcactaagcccggttttcgatatgaagaaaattatatataaacctgctgaagatgatctttacattgaggttattttacatgaattgtcatagaatgagtgacatagatca

SEQ ID NO: 21 - DNA, 500 bp, Saccharomyces cerevisiae
  source 1..500: mol_type = other DNA
tccgaagagcgtgctaccaattcttcatctcgttaacaaactggttctccgttaaaaattgtgctatatgtcctataagccaactctatctatatcttttcttttagtcctactttggatactgttaccaccattttagattgctttttcttttgccgctagccttacaatatttggcaaactttttttttttagccgccgagactcttgatctatggccgggcgaaagggcaaatgactgcttatccccgccatcacttccccccgcccaagggtttagaattggggattaagtaaaaacgaatgactattcctctcaaagtcatccttgttcgacaaaaagaatggaatataacatattggaacaatttcatcctcttttccccattttcgcatataagagcaactaaacgccggtgagtaaagtgcccttccctacagactcttttactcaggtatatatatatatatatcccttaaaaactaaaaagaaagcactc

SEQ ID NO: 22 - DNA, 310 bp, Saccharomyces cerevisiae
  source 1..310: mol_type = other DNA
agcggttgttctaaccactatttaaagccgcaattagtaatgcaaaaagttggccggaattagccgcgcaagttggtggggtcccttaatccgaaaaaggacggctttaacaaatataaactccgaaaatccccacagtgacagaattggagaaacaaccagttttgatatcgccatacatataaagagatgtagaaagcattcttcactgtaatgtccaaatcgtacatttgaatttcttgtaggtttatttaaaaggtaagttaaataaatataatagtacttacaaataaatttggaaccctagaag

SEQ ID NO: 23 - DNA, 340 bp, Saccharomyces cerevisiae
  source 1..340: mol_type = other DNA
aatttttattttctccttccatatgagcgacagcggttactagccgctgtcctcaggttaatgatccaagtccgagatccgggccgaatatgcttgcggggaaagaaataaaagtgcattggagaagaaaaggatatgctcttcaattagaagcgccgaaacactaacatcatgctagcgatatcatacgtacactatataatgtaaaaaatgggcttaagaataactctcttatttcttaacttttgttgcggttgaagagcttataaaagtactagtggcctaaagaagctacagcgccgataataatatcgatttcgacttttctagtatttcgccg

SEQ ID NO: 24 - DNA, 500 bp, Saccharomyces cerevisiae
  source 1..500: mol_type = other DNA
tgtgcacatacgtccagaatgatatcaagataaatggcacgtgtatgtacggctgtgtaaatatgataatcatctcggacgaacggcgtagcactctccatcccctaaaaatgttcacgtgtgactgctccatttcgccggatgtcgagatgacccccccccctcaaaaggcactcacctgttgacatgccgtggcaaatgattggggtcatcctttttttctgttatctctaagatccaaagaaaagtaaaaaaaaaaggttggggtacgaattgccgccgagcctccgatgccattattcaatgggtattgcagttggggtacagttcctcggtggcaaatagttctcccttcattttgtatataaactgggcggctattctaagcatatttctcccttaggttatctggtagtacgttatatcttgttcttatattttctatctataagcaaaaccaaacatatcaaaactactagaaagacattgcccactgtgct

SEQ ID NO: 25 - DNA, 500 bp, Saccharomyces cerevisiae
  source 1..500: mol_type = other DNA
aatataaataaaattccatacagcatgtctaatcatagctaatttatacatattcatcatgaaaacatataggggaaaatatggtcggttaacacacctatcaaaaaattattcagcaattccaatctcgttagtaaaatatattcttattttttttttttttctctgattgtattatttctggagttttgacttatttttttaccacatcgcgcttttcgtccccaatctctctgatatatgatgctgtctataggtagccacttccccgatgtcggacctcgggccgtttacaaactttattgagatgaccttatttctccacattctagtcattcaacttttaccctcatatgtttaccttcactaatgtgaaagcatgaccaaagaaagtgtataaggtatataaatctgccataatgtatgtataacttattaggactttctcaaatagtattttggtattttctactgttctctgatgatcgagagcaaacaga

SEQ ID NO: 26 - DNA, 500 bp, Saccharomyces cerevisiae
  source 1..500: mol_type = other DNA
aagtacgatatggtataactgtaacattgaaggactgaaggactgaaggactgaaggactatagtcaagggccaatggggaaggtcccttccaggccatttgcccgatagtttgtccttctcttgcttttccgacggcccgattgcatgtggcggggcagcactggataaaaaaacgtggggggagtgattaaatttatacgcttattgtgtcaacacggaaaccttatagttatcattactaacatcgcaacaagctgcttttttactcgtttttagccacaccataccccctttaattaactaataatgcataaaatagttattgcttcttgagttgcagcttcttcctggacgtactgttatatatggcatgtcttcgcatgtccgtcaaatttagcgttgtctcgaaacttaggctgtcgttcttgctgtctgtcttctgataaaataatatattggaataagaaaaaaaaaataggaacaagaaagtgtgtgaga

SEQ ID NO: 27 - DNA, 304 bp, Saccharomyces cerevisiae
  source 1..304: mol_type = other DNA
atattattcagttgaaagacaaaaaaacataaatatttctatgagcaaacaatttgaacagaaaaataaaattggggaagtgacacaccatggtagcggttctaaagcgaaatcggcaaagcggctaaatagcagttttgatgacttactccacactgaaaatggatgaccttaaataggagataaagctttttcatccctatgtatttaagatgactggcttgtcaagcattctaatcataaaaaaaagatcgtatttgatcaagaatttatacatagacgccgctaaataattgaatacaaa

SEQ ID NO: 28 - DNA, 500 bp, Saccharomyces cerevisiae
  source 1..500: mol_type = other DNA
ctcgtttgccgttacattgcattgatggtacaataaagggcatgctttatatcgagatgtttcagtgtatatgaggggaaacagaaaagagtcattcctgccattttttggtcactgctttttctgctatgagtaatggtgaagttccttgtggctacacgcttaatgtcatcgggttactgctcctaatatccgcatataagctttatgcagggatcagttgggcggctatttatctacacccagtcatccggcgtgactggatctccacttgccgcaataagtcggtggacaaatggagatttaagagtaaagatgcatgatggtataattcctttagtcgaaatagatatatttcaagcgcatatataggcagacgcttgtactgtagaaatagccgatattcaattgcgctctatgtgtgtttttattccaggttttccttggattctacgtattgtacgactttcttatcctccacaaacgtcatcgtgtcagta

SEQ ID NO: 29 - DNA, 500 bp, Saccharomyces cerevisiae
  source 1..500: mol_type = other DNA
cccaacagatttcaagtctgtcgccttaaccactcggccatagtgcctaaaacaatgtaggttatttaagcaagtattgtagatacttttcgtaataaactacaatgcacccacgactcgcggtgtaatgatggcatgaaatcattgaacgaagttttgcggctatacggctgaaggacgagactaaagggacaggaattattaatgcggggtataatttgaatagtattaacgggcactgccgtttagccatcaaatgctattgttggggtattctctctactttttgttcttggcttgaaccttttcggcggttggcaatcgtccgtatataagcatcggctgtcccaatcctctattgcccttttcccttgcacctccttctcaattcttcgtatctttcgcgtaaaggtagatcttgattcacctatctgtcgaaacacgattaagtgcaaacgaaacaacgtacagtatataacaaagtattttaaataataaga

SEQ ID NO: 30 - DNA, 500 bp, Saccharomyces cerevisiae
  source 1..500: mol_type = other DNA
gctatgacgtttgggtggcctagccggttcgcgtgtgcctgtcgcttttgtcgcttttcaacttctgcccgatatttcctatcaaaggaaaatgggacgttttcaacccctcgctatcatcgtgcctgcactctgcctatcgccaactacaccggggttttatctgcttcacccctccatccagtgctgataacaagaagaaccttgcagggtagggcaggacctacggccaaaatactaattatgtctgtttatgtacatgccccaatctgaatattccatgaatgtaggcacagcatatctccatccatgtactgatacagacgcataaacatatatgtatatacatacttatacactcgaatatttgtagactgatgtacttctatatatatatagggggtttgtgttcctcttcctttcctttttttttctctcttcccttccagtttcttttattctttgctgtttcgaagaatcacaccatcaatgaataaatc

SEQ ID NO: 31 - DNA, 500 bp, Saccharomyces cerevisiae
  source 1..500: mol_type = other DNA
tagatgcgccatctccgagaaaaaatctagacaataacagcgacaattaacctaaagaggatagaagatcgagcaaaaaaatttttttaatatggggtcagtggcgatattatactataggagttaaagagtaagttgagtgtaaggtggtagaattatgattgaactccgaaactaagcgccgattatgggtggcaaagcggacagcttttgatatataatcgatcgctctcgtagttgatatcctctctcttgcttatcttttcctgtatatagtatatgtgtacatacagatacgaatatacctcagttagtttgttttaacattaaatattcaacagttgccagtagcaaaaagaatatatccattcatttcgagctttttcgtctcattactgatatccaactaacagtctcctcatagacggtaccttactttcctttaatattataatactagtatagtcgcacatacttaactcgtctctctctaacacata

SEQ ID NO: 32 - DNA, 500 bp, Saccharomyces cerevisiae
  source 1..500: mol_type = other DNA
ctacgtcgcctgttcgagcggctctgttcgttgcatgaaactaaaataagcggaaagtgtccagccatccactacgtcagaaagaaataatggttgtacactgtttctcggctatataccgtttttggttggttaatcctcgccaggtgcagctattgcgcttggctgcttcgcgatagtagtaatctgagaaagtgcagatcccggtaagggaaacacttttggttcacctttgatagggctttcattggggcattcgtaacaaaaaggaagtagatagagaaattgagaaagcttaagtgagatgttttagcttcaattttgtccccttcaacgctgcttggccttagagggtcagaattgcagttcaggagtagtcacactcatagtatataaacaagccctttattgattttgaataattattttgtatacgtgttctagcatacaagttagaataaataaaaaatagaaaaatagaacatagaaagttttagacc

SEQ ID NO: 33 - DNA, 500 bp, Saccharomyces cerevisiae
  source 1..500: mol_type = other DNA
gagctatagtcttttgcgctttcaatacgtgtagcggtgtaccaaaagttgcacaaaaatgtagttgtcaatgaaagcgcactacgtatataatgactattttttttttcctgggttgcatgggtaatttgttgttaatatgcgattttcttggggaaaagggtgtcatagcgccaaaaactgccgtgcggcacagtatgtatgtttttgagtcgcggcgtttaagggcttggcataaaaagtggttcaagcgagtgataagttgggcgaatgtcgtcttttttgtaaccatgtctttcctgaaaacaacctgtaggcagctccactccacataagggctttctccaatggcaatgggagctcggaacaccggagtagaaatttttataatgtgtattgtataaaacttgcttgttatgcagtttttgttttttttgttactcttccgtagcacaatagacatatattagcggcaaaattgtagtgttgcgattattgcc

SEQ ID NO: 34 - DNA, 500 bp, Saccharomyces cerevisiae
  source 1..500: mol_type = other DNA
gtgtagtattgatcttgttggtattgctagaaatgcttcagcaatactgtataaaatatggaaacgttgccatggcaagacaaaagaagtgatcttgagtgaaataatagagcccggatggccgggtaaattcaaccgctcgtaccgtttataatacgcataaacgccgaaaatgtctctattttagtcattccccagagtgcggtattgcgtacacctgtcatgcgttccttagtgccgatagatatactaatatcgatgcgtcacagtagcagatcatctctgacacttgtttccccatttttttttttcattttttaaagggtttctctacagcctacaggcctcccctaataagtcagcccctccctttggagtgcgctgttgacctgcgtatataagaggtatatcagtgccagtaggtaaacccatcttgcggggattgtaccaggaacatagtagaaagacaaaaacaaccaccgtacttgccattcgtatag

SEQ ID NO: 35 - DNA, 500 bp, Saccharomyces cerevisiae
  source 1..500: mol_type = other DNA
catcaattagggcaaacttgaatagtcagctaggtcatatatttaaaatcaattagccctatgactacattaggtttattgttaggtctttacggctgcatatttgctttcgccgttcggcggggtcctgcgacgatttctgcgcggtcttgtatgggtggagttgacagttaaccctccggaccccctaccccggtgtgcccccggtccatctatccattttgcggtaacccctttgcgcgacagctgcttatcaaggtacctggatcgagccataaaaattgatctacacagatgagatggggcattgggatatattattagtcggagtatcattatagttattcagttttatgcaggttactggccaaacgtttttcttcatttggaataatcgtttaggagctactgttccggtataaagtaacaagcacagtagcagagtaatacgcagtgacgataatagagactagtaaaacagtcgagttgtcggacctaaa

SEQ ID NO: 36 - DNA, 500 bp, Saccharomyces paradoxus
  source 1..500: mol_type = other DNA
tagtcttatctaaaaattgcctttatagtccgtctctccagtcacggcctgtgtaactgattaatcctgcctttctaatcaccattctactgtttaattaagggattttgtcttcatcaacggcttccgcccaaaaaaaagtatgacgttttgcccgcaggcgtgaagctgcccatcttcacgggcctgacctcctctgccggaacaccggccatctccaactcataaattggagaaataagagaatttcagattttcagaggatgaaaaaaaaaaggtagagagcataaaaatggggttcactttttggcaaagttacagtatgcttattacatataaatagagtgccgataatggctttttttcatcttcgaaatacgcttgctactgctcttccagcgtttttattacttctttcttgtttctccttagtatataaaatatcaagctacaacaagcatacaatcaactgtcaactgtcaattatattataatacact

SEQ ID NO: 37 - DNA, 496 bp, Saccharomyces kudriavzevii
  source 1..496: mol_type = other DNA
ctctcaaatcttttagcgccaaggactccaactaattgtatcttgaatttgcctttacgatccgtttgtccagtcacggcatgtatatcttattaatcctgcctttctaatcacgtattctaatgttcaattaagggattttatcttcatcaacggctcccacgcaaaaaatgacgttttgcacacagacacgaaatacaccttccaccggaacaacggccatctccaacttataagttggggaaataagacaatttcagacttcagagaatgaaaaaaaaaaaaggtacatcacagatggggttcaggtttgctacaattgcagggagcctgtcacatataaatagacctccagtgatgatatctttcagtcttcaaacgtctcttgtcacagttctggtcgttctatatcacatctctcttggttctacttattgtctataatatcaagctacagcaagcatacaatcaactatctaccataccataatacaca

SEQ ID NO: 38 - DNA, 441 bp, Saccharomyces bayanus
  source 1..441: mol_type = other DNA
gatccagttctccagtgacacagcctttatctggtcaaacctttctttctaatcacctatgctgatgcttaattaagggatttttgtctccatcaacggcatgcgcccaaaaatgacgttttttttaacccatagacacgaaactacccattttccaccggcctgacctaccaccggaacaacggccatctccaacttgcaagttggggaaattaagagcatcgcaggtttaatggaagaaaaaaaaaaggtacagcacagcgcaaatggagttagttcccttatgtcacacactcacacacagtcggtcagatcaagcatactgggtgcgtataaatagagtggccattgccaccctgtttatctcaaaatctgtcttgttagtggtcttctccctttttcaggttacaattctcttgtttctacttagtatataagtat

SEQ ID NO: 39 - DNA, 480 bp, Saccharomyces mikitae
  source 1..480: mol_type = other DNA
tttcccaaaaagtattatttttaagtgataattgataaaaggggcaaaacgtagacgcaaataaaacggaaataatgattctcagaccttttagcgtcaagaactgcaactaatcttatcttaaaattatctttataatccgtttctcccgtcacagtctgtgtatctgattaatcctgcctttctaatcacctattctaatgttcaattaagggattttgtcttcaccaacggcttccacccaaaagtaaaaaatgacgttgtacccacagacatcttcaccggcctgacctgccaccggaacaacggccatctccaactcataaattggagaaataagagaatttcagattctggaggatgaaaaaaaaaaaggtacagcataaatggggttttatgtgggtacaattacactaggactatcacatataaatagacgggcaatgtaggttcttttccacccttgagacagagttattc

SEQ ID NO: 40 - DNA, 500 bp, Saccharomyces castellii
  source 1..500: mol_type = other DNA
tgtcgtggacgaaatacgccacaattttgccgagaaggtcattagtatgtccaagaaaccctaggtgtaaagtcgggaaatccgaatctccgattttggaggggcccatgccctactttttttcgccaggggtgaaattccaaacccgtgcgcgttcttggaatttgacagcgcattgagtatgtgctgcgtattcccactatcatgacgcgccctttatctgggaaaaatggaactggatgctgaaatatttcactctcagatcacatatcccaaatcctgtgagtgaattgtttggtcaggcgaccaaacaggaatatggaatagattctattctctggattctacaattatccattgttagcaaaacaaaaaaaactggtggtatatatattcagagcctaaaatttaaaggttggatctcaattttaaaagttttcattctgttttgtttttgtttcttcttagctcacgaataaccaaacaaaaaacaatcaata

SEQ ID NO: 41 - DNA, 500 bp, Saccharomyces paradoxus
  source 1..500: mol_type = other DNA
caataggaaaaaaccaagcttcctttcatccggcacggctgtgttgtacatatcactgaagctccgggtattttaagttatacaagagaaatatgcgggctagactagcaagattctggactgtataacgttgtggataggcggataaagggcccaaacaggattgtaaagcttagacgcctctggttgggcaatggcatgtttgtgtattaagtaagacttggctgcgggatagcaaaactgagcagaatatagaaggccacaaaaaaaaggtatataagggcagcaaagtctttataatatatgtagattctcttctctgtgtaattcattcttgtgcttaccactcaaatatacagaagtaagacagataaccaacagcctttcccagatatacatatatctcattgtttcagtttaaacaataatcatatttgtttaactcaaaaataaaaaaaaactaaactcactcaatcaatcattccataaaaaaaaacaat

SEQ ID NO: 42 - DNA, 500 bp, Saccharomyces kudriavzevii
  source 1..500: mol_type = other DNA
cttcctttcatccggcacggctgtgtccccacatctccctaaagctccgggtattttaagttatacaagggaaatatacgggctggactacaacttgcaggttgcacagcgttatggataggcggataaagggcccaagcaagatcgtgaagcttggacgcgtctggttggacaatggtgactttttgtgtattagataatgcttgactggagaatatcaggactgagcagagttaggaagaccacaaaaaaggtatataagggcaacaaagtctccgtgatatggataggctcttctctctggttacaattcattatttcagttgtttgctagatatagagatataatacatctaataaacagtcacttccagagatatatatatatacatatatctatctcctcctcccagcttaaataataactatatttgtttaactcgaagaaaaaaaaaattcaaatttactctatcaattcaattacctcataaaaaacaata

SEQ ID NO: 43 - DNA, 500 bp, Saccharomyces bayanus; identical to SEQ ID NO: 42
  source 1..500: mol_type = other DNA

SEQ ID NO: 44 - DNA, 500 bp, Saccharomyces paradoxus
  source 1..500: mol_type = other DNA
cgataccacacggtccattgggccggtggtgttagtcgacggatatatgcatctgtcccctttcccggcgagccggcagtcgggccgaggttcggataaatttttgcattgtattagtttctgtcatgagtattacttatggttcctttagagctaatcattagctcggtaccggctgttatgcaatttatgacttttcttctacagtgtcagccttgtgacgattatctatgaactttggatgtagcgcatcgagattcgtatctttcattggatagtaaatgggaaggatcgatgacccttattacattctttcctatacttaatatccatttaatctatcttcttgaaagtatataagtaacggtaaatttaccatacttatgctattctcatttatcccctaattttcttttaacttctcgccctacagtaactaagaataacggctactgtttcgaaattaagcaaagtagtaaagcacataaaagaataaagaa

SEQ ID NO: 45 - DNA, 500 bp, Saccharomyces kudriavzevii
  source 1..500: mol_type = other DNA
agaccgaagcgggtaatggacggaattaagcaattgtcccctctcccggggagccgacagtcggaccgagcttcggataaatttctgtattgtttttgtttccgtcatgggtattattttcgggatccttttgccaaccccatagtcaatcgttaacatttaccggccaatatgtaggattatgactattctcctgcatgatcagcggaagtgacgattatctattaattttgaacttctacttcgtgatccggaatttaattggataataatgtgtccgaaggatcgagtgacccttatattctgtagttttttgttactggccatccaattcgtgttcttggaagtatataagttacagtcgattgacctttctcaagctattttcatctttctcctacatttacgtttctcttcttcaatacagcagctagaagttacgattactcctgtgaagataaacaaagtaatagtagcccacaaaaagagagaaagtaaaa

SEQ ID NO: 46 - DNA, 500 bp, Saccharomyces bayanus
  source 1..500: mol_type = other DNA
gtagcagtccggaaataagcaaatgtcccctttcccgagctaaccaacggtcgggccgagcctcggataaatttttgctttgtttttgtttctgtcatgggtattatacatcatttatttagttaacccctagactaattagccggccattagtatgtaagattatgactatagtttgtaccggaaccctggtagcaactactcatgaactttgggctcagtatttcgcaatcccggttttaattggatagcctatcgcgaaggatcgatggatgacccttagaattgtctcttttgttactactcattcaatgcgtgtgctcttgcaagtatataagtcactctaaattagtttatacttgagctttttacatttctcccttgattgtttctttctcttttccccttgttctggtttattgtaatagctaagtgcaacgattaccgctgttaagttaaagaagagagacaagtaataatagtacacagcaaggaaaaaa

SEQ ID NO: 47 - DNA, 500 bp, Saccharomyces paradoxus
  source 1..500: mol_type = other DNA
ttactaaataggctggcatcagctaacccggatggttgaatccggcttttgctacttgttgtccgatgaaaaggagcggcttcccttttgccccagatttccattcatccgagaggtcgcttatcagacttcgtcatttctcatttcatccgagatgatcaaaattgaagccaatcaccacaaaactaacacttaacgtcatgttacactaccctttacagaagaaaatatccatagtccggactaacattccagtatgtgactcaatattggtgcaaatgagaaaatcatagcagtcagcccaagtccgccctttaccagggcaccgtaattcacgaaacgtttctttattatataaaggagctactttactagcaaaattcttgtaattcctcttcccttgctaacttcttcttgttttcttttcctttttacacacagatatataacaattgagagaaaaactctagtataacataacaaaaaagtcaacgaaaaaa

SEQ ID NO: 48 - DNA, 500 bp, Saccharomyces kudriavzevii
  source 1..500: mol_type = other DNA
gttacggtgccgcgccggtggccggtggtcttccggtaaacaaaaaaagctgcctccctttcgccccagatttccattcatccgagggcaccgcttgtcagactttatcgttttcctcatttcatccgagaagatcaattcaaaggcaatgaccacaaaagcaactcctaacgttgtgttacgctaccctttacacaaaatattcataacccgtaatgaatcctaaggtatgtgactcaattttggtgtagaaaatgaggaaaacgtaatactaagttaaagctcgccctttaaagtgaatattccttgaccatttgcgcaggcacacccgaattcacaaacgtttctttattatataaaggaccagctctgctagtcaaatttttataactgcttgttcagttgctgcttctttcttgtcaatttatttcttgtactgttcaactacataaagcaaagagaaaactctcagaataacataacaaagaagtcaacgaaaa

SEQ ID NO: 49 - DNA, 500 bp, Saccharomyces bayanus
  source 1..500: mol_type = other DNA
acgaggctcggcgtttactgctgaatttccggaaagaaagggaaggttccctttaccccagatttccattcatccgaaggactgcttatcagaatttgacatttttctcattttatccgagaagatcaatttaaggctagtgaccacaaaactaactctcatgctgcgctaccgcaagtttcgctcacagaaagaaagcaagcacccatagtccggactacatccttgtatgtgactcaaatttttggcgttgccaattaaactgaagtgtaaagattacttcaagctcaccctttaaagtagaattccttaacggttttaaatagacacaccgaaattaataaacactttctttattatataaaggacagagtttattactggaattctcttaacgccttcctcccttactattgtatcttttcctttcacataatcgctacataactacatagagaaaactctcagattaacacagtaacaacgaagaaaacaaaaaa

SEQ ID NO: 50 - DNA, 500 bp, Saccharomyces cerevisiae
  source 1..500: mol_type = other DNA
acagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 51 - DNA, 412 bp, Saccharomyces cerevisiae
  source 1..412: mol_type = other DNA
atagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 52 - DNA, 500 bp, Saccharomyces cerevisiae
  source 1..500: mol_type = other DNA
tgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaa

SEQ ID NO: 53 - DNA, 800 bp, Saccharomyces cerevisiae
  source 1..800: mol_type = other DNA
catgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaaa

SEQ ID NO: 54 - DNA, 430 bp, Saccharomyces cerevisiae
  source 1..430: mol_type = other DNA
tatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaa

SEQ ID NO: 55 - DNA, 466 bp, Saccharomyces cerevisiae
  source 1..466: mol_type = other DNA
gcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcgcatgccagcattgaaatgatcgaagttcaatgatgaaacggtaattcttctgtcatttactcatctcatctcatcaagttatataattctatacggatgtaatttttcacttttcgtcttgacgtccaccctataatttcaattattgaaccctcac

SEQ ID NO: 56 - DNA, 382 bp, Saccharomyces cerevisiae
  source 1..382: mol_type = other DNA
acaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaaggaggatatcaggtcctatttctgacaaacaatatacaaatttagtttcaaagatgaatcagtgcgcgaaggacataactca

SEQ ID NO: 57 - DNA, 400 bp, Saccharomyces cerevisiae
  source 1..400: mol_type = other DNA
agtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataactacatggatgataagaaaacatggagtacagtcactttgagaaccttcaatcagctggtaacgtcttcgttaattggatactcaaaaaagatggatagcatgaatcacaagatggaaggaaatgcgggccacgaccacagtgatatgcatatgggagatggagatgatacct

SEQ ID NO: 58 - DNA, 500 bp, Saccharomyces cerevisiae
  source 1..500: mol_type = other DNA
ggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctat

SEQ ID NO: 59 - DNA, 1041 bp, Aspergillus tubingensis
  source 1..1041: mol_type = other DNA
atgctgggattcccaatgttcaacccagctacgcctgatgtctggaagatgaataccccttactttccatttgttacaccggggttatttcctgcctcagcacccccatcgcccaccaacgtagatgccgaagctgccagttcccaacagtcggaagcaagctatctggataaggagaaaattgttcgagggccacttgattatcttctcaaatcccctggaaaagacattcgtcggaaattcattcacgcgttcaatgaatggctgcgcattcctgaggacaagttgaatattatcacggaaattgttggattgcttcacacggcctcccttctaatcgacgatattcaggacaattccaagcttcgacgcggcctcccagtggcccatagcatatttggtattgcgcagacaattaactctgccaattatgcgtactttctagcccaggaaaggctccgcgaactgaatcatcctgaagcgtacgaaatatacacagaggaactgcttcgtctgcaccgcggtcaaggtatggacttgtactggcgggactgcctaacctgtcccacagaggaggactatattgagatgatcgccaacaagactggtggcctatttcgactggcgattaagcttatgcagttggaaagcactttgtgcagcaatgtcattgaactagcagacttgttgggcgtgatctttcagattcgggatgattaccaaaacttacagagtggactatacgccaagaacaagggattttgcgaggatttgacggagggaaaattttcctttctgattatccacagtattaacagtaacccgaacaatcaccatctgctaaatatactacggcagcggagcgaggacgattcggtgaagaagtatgctgttgattatatcgactcgacggggagttttgactactgccgggaacggctcgcttccttattggaagaggcggatcaaatggttaagaagttggaaaatgaggggggacaatcaaaggggatctacgatattctgagctttctgtcgtga

SEQ ID NO: 60 - DNA, 732 bp, Aspergillus tubingensis
  source 1..732: mol_type = other DNA
atggatgggttcgaccattctactgctccaccaggatataacgagctaaaatggctcgccgatatcttcgtcatcggaatggctgttggctgggttgctcactatatggagatgattcacacgtcgttcaaggaccaaacatactgcatgaccatcgggggcctttgcatcaattttgcctgggaaatcatattctgcacaatgtatcctgccaaaggatttgtcgagcgggttgcctttctcatgggcatttctctcgaccttggggttatttacgcgggaatcaagaacgccccaaatgaatggcaccactctgcaatggtgagggaccatatgccccttgtcttcgcagcaacgacactttgttgtctgagcggtcatatggctcttactgcccaggttggtcccgcacaagcctatacgtggggggcaattgcatgccagctctttatcagcatagggaatgtgtttcaattgttgagtcggggaaacacacgaggggcgtcatggacgctatggacctccaggttttttggatcaacatcagccattggctttgctcttgttcgatatattcgctggtgggaggccttttcttggttgaactgcccgcttgtgatatggtccgtggccatgttctttctgtttgaaacactctatggagccctattctattctgtcaagcgacaagaagggagatcccagcgtggaatcaagcacaaagagaggtag

SEQ ID NO: 61 - DNA, 1008 bp, Aspergillus tubingensis
  source 1..1008: mol_type = other DNA
atggcggcacttccggacgttgcctccattcccatccctctggtggcaaccctaggcattgcccctctaattttctatctcgtccttgatagaattagccccttgtggccaaattccaaagctttcctgattggcaagaagaaaccggagaccgtgacatcgttcgagtgcccatatgcctacatccgtcagatctatgggaagtatcactgggagccattcgtacagaagctgtctccgaggcttaaggatgaggatccggccaaatataagatggttctggagataatggatgcaatccacctgtgtctgatgctagttgacgatataactgacaatagcgactatcgaaaaggcaagccagcagcccaccggatatatggcccttcagagacagcaaatcgcgcttactaccgagtcacccagattctaaacaagaccgtgcaaaagttccccaagctggccaagttcctgcttcagaatctggaagaaattctcgaaggccaagacctgtcactaatctggcgacgggatggactgggtagcctttcgactgttcctgatgagcgagttgcagcctatcgcaagatggcgtcattgaaaactggggcgttattccggctgctggggcaattggtgatggaggaccaatcgatggacgggacgatgactactcttgcgtggtgctctcagctgcagaatgactgcaagaatgtctactcatctgaatatgctaaggccaaaggggcgcttgccgaagacctccgaaatcgagagctctcatttccaattatcctcgcgctggaagctcctgaagggcattgggtcgccagtgctttggagaccagctcaccgcgcaacattcgcaaggcgcttgctgtgattcagagtgagagagtgcgcaatgcttgtttcaaggagctcaagtcggcgagtgcttcggtccaggactggttggctatttggggacggaacgagaaaatgaacttgaagagccagcagacgtag

SEQ ID NO: 62 - DNA, 1416 bp, Aspergillus tubingensis
  source 1..1416: mol_type = other DNA
atggccaatgcccagcaacccccctttcgcatccttattgtgggcggttctgtcgcaggcctcatccttgcgcactgtctcgaacgcgccaatatagagtacctcatactcgaaaaaggagaagatgttgctccacaagttggtgcctcgataggtatcatgccaaatggcggacggatcctcgagcaactgggcctatttggggagattgagcgtgtgatcgagccgttgcatcaggcgaatatcagctatccagatgggttctgctttagtaacgtctatcctaaggttcttggcgacaggttcggatacccggttgcattcttggaccggcagaagttcctgcagattgcatatgaggggctgagaaagaagcagaatgttctcaccggtaaaagggtagttggactgcgacagtcggatcaagggactgctgtttctgtggctgacgggacagagtatgaggcggatctcgtggttggtgctgatggagtacatagtcgggtgagaagtgagatttggaagatggcggaagagaatcagcctgcatcagtttcgacacgtgaaagaagaagcatgactgttgaatatgtctgcgttttcgggatttcatcagccatcccagggctcgagataagcgaacagatcaacggtattttcgaccatctatccattctaacaatccatggcagacatggtcgcgtgttctggttcgtgatccagaagctggataggaagtacgtctatcctgatgtcccgcgattctcagacgaggatgccgtacagctcttcgatcgggtcaaacacgtgcggttctggaaaaacatctgtgtgggggacttgtggaagaacagagaggtgtcctcgatgacagcgctggaggagggagtgttcgagacatggcatcatgataggatggttttgattggagatagcgttcacaagatgacgcccaactttggccaaggagctaattcagccatcgaggatgctgccgcgctctcttcccttctacatgatctcgtcaacgcccgtggagtttgcaagccatcgaatgtccagattcagcatctcctcaagcagtatcgggagacccgatacactcgcatggtaggcatgtgtcgcaccgcggcttcagtctctcggattcaggcccgagatggcatcctcaacaccgtctttggacgatattgggcaccttatgctggcaacctgcctgctgacctggcatcaaaagtgatggcagatgcagaggttgttacttttctgcccttgccagggcgctcaggaccgggctgggagatgtacagacgaaaggggaagggagggcaggtgcaatgggtgcttataatcttaagcttacttacgattggtggattgtgcatctggctacaaagcaatgcgttgagtagataa

SEQ ID NO: 63 - DNA, 7050 bp, Hypomyces subiculosis
  source 1..7050: mol_type = other DNA
atgccttctaccagcaatccatctcacgtccctgtggccatcatcggcctggcatgccgattcccaggcgaggccacctcaccatcaaaattctgggatcttcttaagaatggacgagatgcctactcaccaaataccgatcgatataacgctgatgccttttaccatcccaaggcaagcaaccgccaaaacgtgctggcaactaagggcggccacttcctcaaacaggacccatacgtttttgacgccgctttctttaacatcacagccgctgaggccatctcctttgaccccaagcagcgaattgccatggaagttgtctacgaggctctagaaaatgccggaaagacactacccaaggtggcgggcacacaaactgcttgctatatcggctcttccatgagtgattaccgagacgctgttgtgcgtgactttggaaacagccccaagtatcatatcctgggaacatgcgaggagatgatttcaaatcgtgtgtcccatttcttggatattcacggccccagtgccaccattcatacagcctgctcatcaagtcttgttgctacacacttggcttgccaaagtttgcaatctggagagtcagaaatggccatcgctggtggtgttggtatgatcatcacccctgatggtaatatgcatcttaacaacttgggattcttgaaccccgagggccactcccggtcatttgatgagaatgctggtggttacggtcgtggtgagggttgcggtatcctcatcctcaagcggctagacagagctctcgaagatggtgattccattcgcgccgtcattcgagcctctggtgtcaactctgatggctggacacagggtgtcaccatgccctccagccaagcccagtctgcccttatcaaatacgtatacgaatcgcatggcctggattatggtgcgactcaatacgttgaggctcacggtactggtaccaaagccggtgatcccgcagagattggcgccctccaccgcacaattggacagggcgcgtccaagtctcgaaggctttggattggcagtgtcaagccaaacattggccatcttgaagccgccgccggtgtggctggtatcattaagggcgtcctgtccatggaacacggcatgattcctccaaacatttacttctccaagcccaaccctgccatccctcttgacgagtggaacatggccgtgcctaccaagttgactccctggcccgccagccaaactggtcgccgtatgagtgtcagcggtttcggtatgggtggtaccaacggccacgtcgtccttgaggcctacaagccccaaggaaagctcaccaacggccataccaacggcatcaccaatggaatccacaagactcgccacagcggcaagaggcttttcgtcctcagcgcccaggatcaagctggcttcaagcgtttgggtaacgccctggtggagcatctcgatgccctgggccctgccgctgccacccctgagttcctcgccaacctctcccacactcttgccgttggcagatctggcttggcttggaggtccagcatcatcgctgagagcgcccctgatcttcgggagaagctggcaactgatccgggtgagggagccgctcgttcttcaggcagcgagccccgtattggattcgtcttcacgggtcaaggtgctcagtgggcccgcatgggcgttgagttgttggagcgccccgtcttcaaggcttccgtgattaagtccgcggagactttgaaggagctcggctgtgaatgggaccctatcgttgagctttccaagcctcaagctgagtctcgacttggtgttcctgaaatctcacagcccatctgcacagtcctacaagtcgccttggttgatgagttgaagcactggggtgtatcaccttccaaggtggtcggtcactccagtggtgaaatcggtgccgcatacagcattggcgctctttctcaccgtgacgctgtcgccgctgcttacttcaggggcaagtcttccaacggagccaagaagcttggtggtggtatgatggctgttgggtgctctcgtgaggacgctgacaagctcctctctgagaccaagctcaagggcggtgttgctaccgtcgcatgtgtcaactccccctccagcgtgaccatctcaggcgatgccactgctctcgaggaactccgagttattctcgaggagaagagtgtgtttgctcgaagactcaaggtcgacgttgcctaccactctgcccacatgaacgctgtctttgccgaatactctgctgcgattgcccacattgagcccgctcaggcagttgaaggtggaccgattatggtctccagtgtcactggtagcgaagtcgactctgagcttctcggcccttactactggacccgtaacttgatctctcccgtcttattcgccgacgctgtcaaggaattggttacccctgctgatggcgacggccaaaacaccgtcgatctcctgattgagattggtcctcacagcgctcttggtggccctgttgagcagattctgtcccataacggcatcaagaatgttgcttacagatctgctcttactcgtggcgagaacgctgttgactgcagcctcaagcttgctggcgagctcttccttctcggcgtgccctttgagttgcaaaaggccaacggtgactctggttctcgcatgctcactaacctacctccttatccttggaaccactccaagtcattccgtgccgactctcgtctccaccgtgagcatctggagcagaaattccctactaggagtctcatcggtgcacctgtccccatgatggcagagagcgagtacacatggcgcaacttcatccgtctcgctgacgagccttggctccgtggtcacactgtcggtaccaccgttctgtttcctggtgccggtatcgtgagcatcatcttggaagctgctcaacagctggtggataccggcaagaccgttcggggcttccgaatgcgcgatgtcaacctcttcgccgccatggctctccccgaggacctggctactgaggttatcatccacatccgacctcaccttatctctactgttggatcaaccgcccccggtggatggtgggagtggactgtttcctcctgcgtcggaactgaccagctgcgagacaatgctcgcggtctggtagccattgactacgaagagagccgcagcgagcagatcaacgccgaggacaaagcgttggttgcttctcaggtcgcggactaccacaagatcctcagcgaatgccctgagcattatgctcatgacaagttctaccagcacatgaccaaggcctcttggagctacggcgagctcttccagggtgtggagaatgtccgtcctggatacggaaagaccatctttgacatcagagtcattgacattggtgagacctttagcaagggacaacttgagcgacctttcctcatcaacgctgccactctcgatgctgtattccagagctggctcggcagtacctacaacaacggtgctttcgagtttgacaagcccttcgttcccacctctattggcgagttggaaatctctgtcaacattcccggtgatggcgactacctcatgccaggccactgccgctctgagcgatacggcttcaacgagttgtctgctgatattgccatcttcgacaaggatctgaagaatgtgttcctttcagtgaaggatttccgaacttccgagcttgatatggattccggcaagggagacggagatgccgctcacgtcgaccctgccgatatcaactcggaggttaagtggaactacgctcttggcctcctcaagtccgaggaaatcaccgagctggtcaccaaggtcgccagcaatgacaagctcgccgagcttctccgtctgacacttcacaacaaccctgctgccactgtcatcgagcttgtttctgatgagagcaagatctctggcgcatcttctgccaagctgtccaagggccttatcctccccagccagatccgttacgtagttgtcaaccctgaggcagcggacgccgactctttcttcaaattcttctcccttggtgaggatggtgcccctgtcgctgctgaaaggggccccgccgaactgttgatcgcctccagcgaagtcactgacgcggctgtccttgagcgcctgattaccttggccaagcctgatgccagcattcttgttgctgtcaacaacaagactaccgccgctgccctctcagccaaggcgttccgtgttgtcaccagcatccaggacagcaagtccattgctctctacactagcaagaaggcgcctgccgccgacacctccaagctcgaggccatcatcctcaagccaaccactgctcaacctgccgcccagaatttcgcctccatcctccagaaggcactcgagctccagggctactctgtcgtttctcagccatggggcaccgacatcgacgtcaacgatgccaagggaaagacctacatttctctgttggagcttgagcagcctctgctcgacaacctctccaagtccgacttcgagaacctccgcgcagtcgttttgaactgcgagcgtctcctgtgggtcacagcaggtgacaacccatctttcggcatggttgatggtttcgctcgctgcatcatgagcgaaattgccagcaccaagttccaggtcctgcatttgagcgctgcaactggtctgaagtacggatcttctctcgccacccgcattctccagtcggatagcaccgacaacgagtaccgggaggtcgatggtgctctccaggtggcccgtatcttcaagagctacaacgagaacgagagtctccgccaccacctcgaggataccaccagcgttgtgactcttgctgaccaggaggatgctctgcgcctcactattggcaagcctggtcttttggatactttgaagtttgtccccgatgagcgtatgctcccacctctccaggatcacgaggttgaaatccaggtcaaggctactggtctgaacttccgagacatcatggcttgcatgggtcttattcctgttcgatctctgggccaggaggccagtggcatcgtcctcagaaccggtgcgaaggctaccaacttcaagcctggcgaccgtgtttgcaccatgaacgtcggaacacatgccaccaagatccgagccgactaccgtgtcatgacaaagatccccgactccatgacctttgaagaagctgcctcggttgctgttgttcacaccaccgcctactacgccttcatcaccatcgccaagcttcgcaagggccagtccgtcttgatccacgccgccgctggtggtgttggccaagcagccattcagttggccaagcatctcggcctcatcacctatgttaccgtaggtactgaagacaagcgccagctcattcgggagcagtatggcattcccgacgagcacatcttcaactcccgtgatgccagcttcgtcaagggtgtccagcgtgttaccaacggtcgcggtgtcgactgcgttctcaactctctatccggtgagctcctgcgtgcttcttggggatgccttgctacctttggtcatttcatcgaaattggtctccgtgatatcaccaacaacatgcgtcttgacatgcgacctttccgcaagagcacctccttcacattcatcaacacccacactctcttcgaggaagaccccgctgcgttgggagatattctcaacgagtccttcaagctcatgttcgctggcgcccttaccgctcctagccccttgaatgcctatcccattggccaggtcgaggaggccttccgaaccatgcagcagggcaagcaccgcggtaagatggtgctgtccttctccgatgacgcaaaggctcccgtgttgcgcaaagcgaaggattccttgaaactggaccctgacgccacttacctctttgttggtggtcttggtggtctgggtcgcagtcttgccaaggagtttgttgcgtctggcgcccgcaacattgccttcttatcccgatccggtgacactaccgcccaggccaaggctatcgtggacgaattggctggccagggtatccaggtcaaggcctatcgtggtgatatcgccagcgaggcatccttcctccaggctatggagcaatgctctcaggatctcccgcccgtaaagggtgtgatccagatggccatggttctccgcgatatcgtctttgagaagatgtcgtacgatgagtggaccgtccccgttggccccaaggtccaaggttcatggaacttgcacaagtacttcagtcatgagcgacctcttgacttcatggtcatctgctcctcaagctccggtatctacggttatcccagtcaggctcaatacgccgctggcaacacttaccaggatgccttggctcactaccgtcgctctcagggcctgaacgccatctccgtcaacttgggtatcatgcgagatgtcggtgtcctggctgagacgggtaccactggtaacatcaagctctgggaagaggtcttgggcatccgcgagcctgccttccacgctctcatgaagagcttgatcaaccatcagcagcgtgggtctggggactacccggcgcaggtctgcactggtcttggtactgctgacattatggctactcacggcctggcccggcccgagtatttcaatgacccccgttttggaccccttgccgtcaccactgtcgcgaccgatgcttcagctgacggccagggctctgctgtctcgctcgcctctaggctctccaaggtttccaccaaggatgaagctgccgagatcattaccgatgctctggtcaacaagacggcagacatcctgcagatgcccccctctgaagtcgaccccggccgacctctgtaccgttatggtgttgactcccttgtggcgcttgaggtgcgaaactggatcacaagggagatgaaggcgaacatggcgctgctggagattctggcagccgtccccattgagagcttcgctgtcaagattgctgagaagagcaagttggttactgtttaa

SEQ ID NO: 64 - DNA, 6150 bp, Hypomyces subiculosis
  source 1..6150: mol_type = other DNA
atggtgactgtaccacagactatcctctactttggagatcagacagactcctgggttgattccctcgatcagctatacagacaagccgctacgataccatggctacagacgtttctcgacgaccttgtaaaggtcttcaaggaagagtcccggggcatggatcatgcgttacaagacagtgttggtgaatactctacactactcgacttggcggatagataccgccatggcaccgacgagattggtatggtgcgtgctgtcttgctacatgccgcgagaggaggcatgctattacaatgggtgaagaaagaatcacagcttgtggacctcaatggctccaagcctgaagcactcggtatctctggaggactcaccaacctcgcagcactggcgatatccacagacttcgagtctctatatgacgcagtcattgaggctgcgagaatatttgtcagattatgccgttttacttcggtacgatcaagagctatggaggaccgacctggcgtttggggctgggcagtgctgggaattacaccagaggaactgagcaaagtgcttgagcagttccaatccagcatggggattcctgccatcaagagagctaaggttggcgtaacaggagaccgatggagcaccgttattgggccaccctcagtcttggacctattcatccaccagtgtcccgctgtgcgcaacctccccaagaatgaattgagcatccacgcccttcagcacacagtcacagtcacagaggctgacctcgacttcattgtcgggagtgctgagcttcttagtcaccccattgtgccagacttcaaagtctggggaatggatgatcctgtggcatcctaccagaactggggagaaatgctaagagcaatcgtcactcaagttttgtccaagcctttggacattaccaaggtgattgcgcaactcaacactcacctcggccctcgtcatgtcgacgtccgagtcatcggacctagcagccacaccccctacttggcgagttcgctcaaagctgctggcagcaaggctattttccagaccgataagactcttgagcagttacagccgaagaaactccccccgggccgcatcgccattgtcggtatggctggccgtggtcctggctgcgagaatgttgatgagttctgggacgtcattatggcgaagcaggatcgttgtgaagagattcccaaagatcgcttcgacatcaatgagttctactgtaccgagcacggggagggttgcaccaccaccacaaaatacggctgcttcatgaacaagcctggaaactttgactcccgcttcttccacgtgtcgcctcgtgaggcgctgttgatggaccccggtcacaggcagttcatgatgagcacttatgaagctcttgagacggcaggatactctgatggccagactagggacgttgatcctaataggatcgcggcgttctatggccagtccaacgatgattggcatatggtgagccattataccctgggttgtgatgcctacaccctgcagggggcgcaaagagccttcggcgctggtcgcatcgccttccacttcaagtgggagggcccaacatactcgctcgattctgcatgtgcctccacctcctctgctattcacctggcctgcgtgagtcttctatccaaagatgtggacatggctgttgtgggtgctgccaacgtcgtcgggtatcctcactcctggacaagtcttagcaagtctggtgtcttgtccgacactggaaactgcaaaacctactgcgatgatgctgatggttactgccgagcagactttgtcggctcagttgtgctgaagcgtctcgaagatgctgtcgagcaaaacgacaacatcttggctgtcgtggctggttcaggcagaaaccactccggcaactcttcatccatcaccacgtcggatgccggtgcccaggagagactgtttcacaagattatgcacagcgccagagtctctcctgatgagatctcatatgttgagatgcacggcactggaactcagattggcgatccggccgagatgagtgctgttaccaatgtcttcaggaagaggaaggcgaataaccccctaactgttggtggaatcaaagcgaacgtcgggcatgctgaagcttctgctggcatggcctccctgctcaaatgcatacagatgttccagaaagatattatgccccctcaggctcgaatgccccatactctcaacccaaagtatccgagtctttctgagcttaacattcatatcccctccgagccgaaggagttcaaggctatcggcgagcggccacgacgcatcctccttaataactttgacgcagcaggtggcaacgcctctctcattctggaagacttcccctccaccgtcaaggaaaatgcggaccccaggccaagccatgtcatcgtttcctctgccaaaacacaatcctcatatcacgcgaataagcgtaacctcctgaagtggctacgcaagaacaaagatgctaaactcgaagatgttgcatacacaaccaccgcccgcagaatgcaccaccccctcagattctcttgcagtgcctccacaacggaggagctcatttccaagcttgaggcagacacggcagatgcaactgcgtctcggggctcgcccgttgtcttcgtattcacgggacagggctctcactacgccggcatgggtgccgagttgtacaagacatgccctgctttccgcgaggaagtcaacctctgtgccagcatctctgaggagcacgggttccccccgtacgtggatatcatcaccaacaaagatgttgacataaccaccaaggacaccatgcagacacagctcgctgttgtcacgctggagatcgccctcgccgcattctggaaggcgtctggtatccagccgtcagcagtcatgggtcactccctgggcgagtatgtggctctccaggtcgcaggggtcctatctctagctgatctgctctacctcgtcggcaatcgggcccgtctcctgctggagcgctgcgaagccgacacctgcgctatgttggcagtatcaagctctgctgcctccatccgcgagctcatcgaccagcgcccgcagtcatccttcgagattgcatgcaagaatagccccaatgccacggttatcagcggcagcactgatgagatttctgagctccagtcatccttcacggcatcacgagccagggctctgtctgtgccctatggatttcactccttccagatggatcccatgctcgaggattacatcgttcttgcgggtggtgtaacctactcgccaccaaagattccagttgcttcaaccctgctcgcttcgattgtggagtcttcaggggtcttcaacgcttcctacctcggtcagcaaacccgccaagctgtcgacttcgtcggtgctcttggcgccttgaaggagaagtttgctgaccctctctggctggagatcggacccagccaaatctgcagctcctttgtccgggcgactctctcaccctcgccgggcaaaatcttgtccactttggaggcaaataccaacccctgggcatccatttccaagtgcctcgccggcgcgtacaaggatggtgtcgcagttgactggttggcggtgcatgctccattcaagggcggcttgaagctcgtgaagttgcccgcctatgcatgggacctcaaggacttctggattgtctactctgaggccaacaaggctgctcgagctttggctcccgctccctcgttcgaaacacagaggatttctacatgtgctcaacagattgttgaagaatcatcatcacccagcctccatgtctctgcccgagctgctatctccgatcctggcttcatggccttggtcgacggtcatcgcatgcgcgatgtgtccatctgccccggaagtgtcttctgcgaggcaggccttgccgtctccaagtacgcactgaagtacagtggccgaaaggataccgtggaaacaagacttacaatcaacaacctgtctctcaagcgcccgctcacaaagtctcttgtaggcaccgatggcgagcttctcaccacggttgttgcagacaaggcctccagcgataccttgcaggtttcatggaaggcttcttcctctcatgcatcatacgatcttggtagctgcgagatcaccatttgtgatgcccagactcttcaaactagctggaacagaagctcatacttcgtcaaggctcgtatgaacgagttgatcaagaatgtcaagagcggaaatggtcaccgcatgctccccagtatcctctacactctcttcgctagcacagttgattatgaccctaccttcaagtctgtcaaggaggccttcatctcaaatgagtttgacgaagctgctgcggaggtggtgcttcagaagaacccggctggaactcagttctttgcgtccccttactggggtgagagcgtagttcatcttgccggtttcctcgtgaactccaaccctgcccgcaagactgcttctcagacgaccttcatgatgcagagtcttgagagcgtcgagcagaccgctgatctcgaggctggacgcacttactacacctatgctcgcgttttgcatgaggaagaagacacagtcagctgtgacttgttcgtcttcgactcggagaagatggtaatgcagtgctcgggactctcattccatgaggtcagcaacaatgttctggacagacttcttggaaaggcatcaccgcctgtgaagcaagtttcccaccagaaggcgccagtgcttgtgcccgcagagtcaaaaccggccctgaaagctgctgtcgaggcggctcccaaggcgcctgagcctgtgaagacagaggtgaagaagatctcttcgtcggagagcgaattgttccacactattcttgaaagcatcgccaaggagactggcactcaggtctctgacttcactgatgacatggaactggctgaacttggcgttgattccatcatgggtattgagatcgctgccggcgtcagcagcagaaccggcctcgatgttctcctcccctcttttgtcgtagattatcccaccattggagatctgcgaaacgaatttgcgcgctcctctacatctacacctcccagcaagaccttttccgagttctccatcgtcgatgccactccagagtctacgcgcagctcgagtcgagcgccttctgagaagaaggagcctgctccggcttcagagaagtctgaggagctggtgatcgttccgtccgcggttgtcgaggattcctctcccctccccagtgccagaatcaccttgatccagggtcgatcttcgagtggaaagcagcctttctacttgatcgccgatggagctggtagcattgctacgtatatccacctggctcccttcaaggacaagagaccggtttatggcattgattcgcctttcctccgttgccccagcaggctgaccacccaggtgggcattgaaggcgtcgcaaagatcatctttgaggcgttgattaagtgccagcctgagggtccctttgacttgggaggattctctggcggagctatgctcagctatgaggtgtctcgccaactcgctgccgccggtcgcgtcgtctccagtcttctcctcatcgatatgtgttctccccgtcctttgggtgttgaggacacaatcgaggtcggctggaaggtctacgagaccatcgcttcccaagataagctctggaacgcctcaagtaacacccagcagcatctcaaggccgtcttcgcctgcgtcgcagcctaccaccctcctcccatgactcccgctcaacgacccaagcgaacagctatcatctgggctaaaaagggcatggtcgaccgttgttctcgcgacgagaaggtgatgaagttcctggccgacaagggcatccccaccgagtcgtacccagggttcatggaggaccccaagctgggtgccgtggcgtggggccttccgcacaagtccgctgcggacttgggacccaacggatgggacaagttccttggcgagactctgtgcctgtctatcgattcggaccacttggatatgccgatgccggggcatgtgcacttgcttcaggcggcgatggaggagtcgttcaaatatttcagcgaggcaaattag

SEQ ID NO: 65 - DNA, 14802 bp, synthetic construct
  misc_feature 1..14802: note = synthesized in lab
  source 1..14802: mol_type = other DNA
tatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacacaatgctgggattcccaatgttcaacccagctacgcctgatgtctggaagatgaataccccttactttccatttgttacaccggggttatttcctgcctcagcacccccatcgcccaccaacgtagatgccgaagctgccagttcccaacagtcggaagcaagctatctggataaggagaaaattgttcgagggccacttgattatcttctcaaatcccctggaaaagacattcgtcggaaattcattcacgcgttcaatgaatggctgcgcattcctgaggacaagttgaatattatcacggaaattgttggattgcttcacacggcctcccttctaatcgacgatattcaggacaattccaagcttcgacgcggcctcccagtggcccatagcatatttggtattgcgcagacaattaactctgccaattatgcgtactttctagcccaggaaaggctccgcgaactgaatcatcctgaagcgtacgaaatatacacagaggaactgcttcgtctgcaccgcggtcaaggtatggacttgtactggcgggactgcctaacctgtcccacagaggaggactatattgagatgatcgccaacaagactggtggcctatttcgactggcgattaagcttatgcagttggaaagcactttgtgcagcaatgtcattgaactagcagacttgttgggcgtgatctttcagattcgggatgattaccaaaacttacagagtggactatacgccaagaacaagggattttgcgaggatttgacggagggaaaattttcctttctgattatccacagtattaacagtaacccgaacaatcaccatctgctaaatatactacggcagcggagcgaggacgattcggtgaagaagtatgctgttgattatatcgactcgacggggagttttgactactgccgggaacggctcgcttccttattggaagaggcggatcaaatggttaagaagttggaaaatgaggggggacaatcaaaggggatctacgatattctgagctttctgtcgtgagcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcgcatgccagcattgaaatgatcgaagttcaatgatgaaacggtaattcttctgtcatttactcatctcatctcatcaagttatataattctatacggatgtaatttttcacttttcgtcttgacgtccaccctataatttcaattattgaaccctcacgatccagttctccagtgacacagcctttatctggtcaaacctttctttctaatcacctatgctgatgcttaattaagggatttttgtctccatcaacggcatgcgcccaaaaatgacgttttttttaacccatagacacgaaactacccattttccaccggcctgacctaccaccggaacaacggccatctccaacttgcaagttggggaaattaagagcatcgcaggtttaatggaagaaaaaaaaaaggtacagcacagcgcaaatggagttagttcccttatgtcacacactcacacacagtcggtcagatcaagcatactgggtgcgtataaatagagtggccattgccaccctgtttatctcaaaatctgtcttgttagtggtcttctccctttttcaggttacaattctcttgtttctacttagtatataagtatatcaagctatattaagcatactatcaactgtcaactctatcctcaaaatacaatacaaaatggatgggttcgaccattctactgctccaccaggatataacgagctaaaatggctcgccgatatcttcgtcatcggaatggctgttggctgggttgctcactatatggagatgattcacacgtcgttcaaggaccaaacatactgcatgaccatcgggggcctttgcatcaattttgcctgggaaatcatattctgcacaatgtatcctgccaaaggatttgtcgagcgggttgcctttctcatgggcatttctctcgaccttggggttatttacgcgggaatcaagaacgccccaaatgaatggcaccactctgcaatggtgagggaccatatgccccttgtcttcgcagcaacgacactttgttgtctgagcggtcatatggctcttactgcccaggttggtcccgcacaagcctatacgtggggggcaattgcatgccagctctttatcagcatagggaatgtgtttcaattgttgagtcggggaaacacacgaggggcgtcatggacgctatggacctccaggttttttggatcaacatcagccattggctttgctcttgttcgatatattcgctggtgggaggccttttcttggttgaactgcccgcttgtgatatggtccgtggccatgttctttctgtttgaaacactctatggagccctattctattctgtcaagcgacaagaagggagatcccagcgtggaatcaagcacaaagagaggtagacaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaaggaggatatcaggtcctatttctgacaaacaatatacaaatttagtttcaaagatgaatcagtgcgcgaaggacataactcaataggaaaaaaccgagcttcctttcatccggcgcggctgtgttctacatatcactgaagctccgggtattttaagttatacaagggaaagatgccggctagactagcaagttttaggctgcttaacattatggataggcggataaagggcccaaacaggattgtaaagcttagacgcttctggttggacaatggtacgtttgtgtattaagtaaggcttggctggggatagcaacattgggcagagtatagaagaccacaaaaaaaaggtatataagggcagagaagtctttgtaatgtgtgtaacttctcttccatgtgtaatcagtatttctacttacttcttaaatatacagaagtaagacagataaccaacagcctttcccagatatacatatatatctttatttcagcttaaacaataattatatttgtttaactcaaaaataaaaaaaaaaaaccaaactcacgcaactaattattccataataaaataacaacatggcggcacttccggacgttgcctccattcccatccctctggtggcaaccctaggcattgcccctctaattttctatctcgtccttgatagaattagccccttgtggccaaattccaaagctttcctgattggcaagaagaaaccggagaccgtgacatcgttcgagtgcccatatgcctacatccgtcagatctatgggaagtatcactgggagccattcgtacagaagctgtctccgaggcttaaggatgaggatccggccaaatataagatggttctggagataatggatgcaatccacctgtgtctgatgctagttgacgatataactgacaatagcgactatcgaaaaggcaagccagcagcccaccggatatatggcccttcagagacagcaaatcgcgcttactaccgagtcacccagattctaaacaagaccgtgcaaaagttccccaagctggccaagttcctgcttcagaatctggaagaaattctcgaaggccaagacctgtcactaatctggcgacgggatggactgggtagcctttcgactgttcctgatgagcgagttgcagcctatcgcaagatggcgtcattgaaaactggggcgttattccggctgctggggcaattggtgatggaggaccaatcgatggacgggacgatgactactcttgcgtggtgctctcagctgcagaatgactgcaagaatgtctactcatctgaatatgctaaggccaaaggggcgcttgccgaagacctccgaaatcgagagctctcatttccaattatcctcgcgctggaagctcctgaagggcattgggtcgccagtgctttggagaccagctcaccgcgcaacattcgcaaggcgcttgctgtgattcagagtgagagagtgcgcaatgcttgtttcaaggagctcaagtcggcgagtgcttcggtccaggactggttggctatttggggacggaacgagaaaatgaacttgaagagccagcagacgtagagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataactacatggatgataagaaaacatggagtacagtcactttgagaaccttcaatcagctggtaacgtcttcgttaattggatactcaaaaaagatggatagcatgaatcacaagatggaaggaaatgcgggccacgaccacagtgatatgcatatgggagatggagatgatacctccattgggccgatgaagttagtcgacggatagaagcggttgtcccctttcccggcgagccggcagtcgggccgaggttcggataaattttgtattgtgttttgattctgtcatgagtattacttatgttctctttaggtaaccccaggttaatcaatcacagtttcataccggctagtattcaaattatgacttttcttctgcagtgtcagccttacgacgattatctatgagctttgaatatagtttgccgtgattcgtatctttaattggataataaaatgcgaaggatcgatgacccttattattatttttctacactggctaccgatttaactcatcttcttgaaagtatataagtaacagtaaaatataccgtacttctgctaatgttatttgtcccttatttttcttttcttgtcttatgctatagtacctaagaataacgactattgttttgaactaaacaaagtagtaaaagcacataaaagaattaagaaaatggccaatgcccagcaacccccctttcgcatccttattgtgggcggttctgtcgcaggcctcatccttgcgcactgtctcgaacgcgccaatatagagtacctcatactcgaaaaaggagaagatgttgctccacaagttggtgcctcgataggtatcatgccaaatggcggacggatcctcgagcaactgggcctatttggggagattgagcgtgtgatcgagccgttgcatcaggcgaatatcagctatccagatgggttctgctttagtaacgtctatcctaaggttcttggcgacaggttcggatacccggttgcattcttggaccggcagaagttcctgcagattgcatatgaggggctgagaaagaagcagaatgttctcaccggtaaaagggtagttggactgcgacagtcggatcaagggactgctgtttctgtggctgacgggacagagtatgaggcggatctcgtggttggtgctgatggagtacatagtcgggtgagaagtgagatttggaagatggcggaagagaatcagcctgcatcagtttcgacacgtgaaagaagaagcatgactgttgaatatgtctgcgttttcgggatttcatcagccatcccagggctcgagataagcgaacagatcaacggtattttcgaccatctatccattctaacaatccatggcagacatggtcgcgtgttctggttcgtgatccagaagctggataggaagtacgtctatcctgatgtcccgcgattctcagacgaggatgccgtacagctcttcgatcgggtcaaacacgtgcggttctggaaaaacatctgtgtgggggacttgtggaagaacagagaggtgtcctcgatgacagcgctggaggagggagtgttcgagacatggcatcatgataggatggttttgattggagatagcgttcacaagatgacgcccaactttggccaaggagctaattcagccatcgaggatgctgccgcgctctcttcccttctacatgatctcgtcaacgcccgtggagtttgcaagccatcgaatgtccagattcagcatctcctcaagcagtatcgggagacccgatacactcgcatggtaggcatgtgtcgcaccgcggcttcagtctctcggattcaggcccgagatggcatcctcaacaccgtctttggacgatattgggcaccttatgctggcaacctgcctgctgacctggcatcaaaagtgatggcagatgcagaggttgttacttttctgcccttgccagggcgctcaggaccgggctgggagatgtacagacgaaaggggaagggagggcaggtgcaatgggtgcttataatcttaagcttacttacgattggtggattgtgcatctggctacaaagcaatgcgttgagtagataaggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagattaattaa

SEQ ID NO: 66 - DNA, 14644 bp, synthetic construct
  misc_feature 1..14644: note = synthesized in lab
  source 1..14644: mol_type = other DNA
atagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatgctgggattcccaatgttcaacccagctacgcctgatgtctggaagatgaataccccttactttccatttgttacaccggggttatttcctgcctcagcacccccatcgcccaccaacgtagatgccgaagctgccagttcccaacagtcggaagcaagctatctggataaggagaaaattgttcgagggccacttgattatcttctcaaatcccctggaaaagacattcgtcggaaattcattcacgcgttcaatgaatggctgcgcattcctgaggacaagttgaatattatcacggaaattgttggattgcttcacacggcctcccttctaatcgacgatattcaggacaattccaagcttcgacgcggcctcccagtggcccatagcatatttggtattgcgcagacaattaactctgccaattatgcgtactttctagcccaggaaaggctccgcgaactgaatcatcctgaagcgtacgaaatatacacagaggaactgcttcgtctgcaccgcggtcaaggtatggacttgtactggcgggactgcctaacctgtcccacagaggaggactatattgagatgatcgccaacaagactggtggcctatttcgactggcgattaagcttatgcagttggaaagcactttgtgcagcaatgtcattgaactagcagacttgttgggcgtgatctttcagattcgggatgattaccaaaacttacagagtggactatacgccaagaacaagggattttgcgaggatttgacggagggaaaattttcctttctgattatccacagtattaacagtaacccgaacaatcaccatctgctaaatatactacggcagcggagcgaggacgattcggtgaagaagtatgctgttgattatatcgactcgacggggagttttgactactgccgggaacggctcgcttccttattggaagaggcggatcaaatggttaagaagttggaaaatgaggggggacaatcaaaggggatctacgatattctgagctttctgtcgtgagcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcgcatgccagcattgaaatgatcgaagttcaatgatgaaacggtaattcttctgtcatttactcatctcatctcatcaagttatataattctatacggatgtaatttttcacttttcgtcttgacgtccaccctataatttcaattattgaaccctcactgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatggatgggttcgaccattctactgctccaccaggatataacgagctaaaatggctcgccgatatcttcgtcatcggaatggctgttggctgggttgctcactatatggagatgattcacacgtcgttcaaggaccaaacatactgcatgaccatcgggggcctttgcatcaattttgcctgggaaatcatattctgcacaatgtatcctgccaaaggatttgtcgagcgggttgcctttctcatgggcatttctctcgaccttggggttatttacgcgggaatcaagaacgccccaaatgaatggcaccactctgcaatggtgagggaccatatgccccttgtcttcgcagcaacgacactttgttgtctgagcggtcatatggctcttactgcccaggttggtcccgcacaagcctatacgtggggggcaattgcatgccagctctttatcagcatagggaatgtgtttcaattgttgagtcggggaaacacacgaggggcgtcatggacgctatggacctccaggttttttggatcaacatcagccattggctttgctcttgttcgatatattcgctggtgggaggccttttcttggttgaactgcccgcttgtgatatggtccgtggccatgttctttctgtttgaaacactctatggagccctattctattctgtcaagcgacaagaagggagatcccagcgtggaatcaagcacaaagagaggtagacaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaaggaggatatcaggtcctatttctgacaaacaatatacaaatttagtttcaaagatgaatcagtgcgcgaaggacataactcaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggcggcacttccggacgttgcctccattcccatccctctggtggcaaccctaggcattgcccctctaattttctatctcgtccttgatagaattagccccttgtggccaaattccaaagctttcctgattggcaagaagaaaccggagaccgtgacatcgttcgagtgcccatatgcctacatccgtcagatctatgggaagtatcactgggagccattcgtacagaagctgtctccgaggcttaaggatgaggatccggccaaatataagatggttctggagataatggatgcaatccacctgtgtctgatgctagttgacgatataactgacaatagcgactatcgaaaaggcaagccagcagcccaccggatatatggcccttcagagacagcaaatcgcgcttactaccgagtcacccagattctaaacaagaccgtgcaaaagttccccaagctggccaagttcctgcttcagaatctggaagaaattctcgaaggccaagacctgtcactaatctggcgacgggatggactgggtagcctttcgactgttcctgatgagcgagttgcagcctatcgcaagatggcgtcattgaaaactggggcgttattccggctgctggggcaattggtgatggaggaccaatcgatggacgggacgatgactactcttgcgtggtgctctcagctgcagaatgactgcaagaatgtctactcatctgaatatgctaaggccaaaggggcgcttgccgaagacctccgaaatcgagagctctcatttccaattatcctcgcgctggaagctcctgaagggcattgggtcgccagtgctttggagaccagctcaccgcgcaacattcgcaaggcgcttgctgtgattcagagtgagagagtgcgcaatgcttgtttcaaggagctcaagtcggcgagtgcttcggtccaggactggttggctatttggggacggaacgagaaaatgaacttgaagagccagcagacgtagagtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaataactacatggatgataagaaaacatggagtacagtcactttgagaaccttcaatcagctggtaacgtcttcgttaattggatactcaaaaaagatggatagcatgaatcacaagatggaaggaaatgcgggccacgaccacagtgatatgcatatgggagatggagatgataccttatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaatggccaatgcccagcaacccccctttcgcatccttattgtgggcggttctgtcgcaggcctcatccttgcgcactgtctcgaacgcgccaatatagagtacctcatactcgaaaaaggagaagatgttgctccacaagttggtgcctcgataggtatcatgccaaatggcggacggatcctcgagcaactgggcctatttggggagattgagcgtgtgatcgagccgttgcatcaggcgaatatcagctatccagatgggttctgctttagtaacgtctatcctaaggttcttggcgacaggttcggatacccggttgcattcttggaccggcagaagttcctgcagattgcatatgaggggctgagaaagaagcagaatgttctcaccggtaaaagggtagttggactgcgacagtcggatcaagggactgctgtttctgtggctgacgggacagagtatgaggcggatctcgtggttggtgctgatggagtacatagtcgggtgagaagtgagatttggaagatggcggaagagaatcagcctgcatcagtttcgacacgtgaaagaagaagcatgactgttgaatatgtctgcgttttcgggatttcatcagccatcccagggctcgagataagcgaacagatcaacggtattttcgaccatctatccattctaacaatccatggcagacatggtcgcgtgttctggttcgtgatccagaagctggataggaagtacgtctatcctgatgtcccgcgattctcagacgaggatgccgtacagctcttcgatcgggtcaaacacgtgcggttctggaaaaacatctgtgtgggggacttgtggaagaacagagaggtgtcctcgatgacagcgctggaggagggagtgttcgagacatggcatcatgataggatggttttgattggagatagcgttcacaagatgacgcccaactttggccaaggagctaattcagccatcgaggatgctgccgcgctctcttcccttctacatgatctcgtcaacgcccgtggagtttgcaagccatcgaatgtccagattcagcatctcctcaagcagtatcgggagacccgatacactcgcatggtaggcatgtgtcgcaccgcggcttcagtctctcggattcaggcccgagatggcatcctcaacaccgtctttggacgatattgggcaccttatgctggcaacctgcctgctgacctggcatcaaaagtgatggcagatgcagaggttgttacttttctgcccttgccagggcgctcaggaccgggctgggagatgtacagacgaaaggggaagggagggcaggtgcaatgggtgcttataatcttaagcttacttacgattggtggattgtgcatctggctacaaagcaatgcgttgagtagataaggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagattaattaa

(19) United States
(12) Patent Application Publication
(10) Pub. No.: US 2024/0102030 A1
(43) Pub. Date: Mar. 28, 2024

(54) Inducible Production-Phase Promoters for Coordinated Heterologous Expression in Yeast

(71) Applicant: The Board of Trustees of the Leland Stanford Junior University, Stanford, CA (US)

(72) Inventors: Colin Harvey, Mountain View, CA (US); Ulrich Schlecht, Sunnyvale, CA (US); Maureen Elizabeth Hillenmeyer, Palo Alto, CA (US)

(73) Assignee: The Board of Trustees of the Leland Stanford Junior University, Stanford, CA (US)

(21) Appl. No.: 18/462,158

(22) Filed: Sep. 6, 2023

Related U.S. Application Data
(63) Continuation of application No. 16/796,851, filed on Feb. 20, 2020, now Pat. No. 11,795,464.
Application No. 16/796,851 is a continuation of application No. 15/469,452, filed on Mar. 24, 2017, now Pat. No. 10,612,032.
(60) Provisional application No. 62/313,108, filed on Mar. 24, 2016.

(51) Int. Cl. C12N 15/81; C12N 15/63

(52) U.S. Cl. CPC C12N 15/81; C12N 15/635

(57) ABSTRACT

Inducible promoters for the coordinated expression of at least one heterologous gene in yeast and methods of using them are disclosed. In particular, the invention relates to sets of inducible promoters derived from S. cerevisiae and related species that can be induced in the presence of nonfermentable carbon sources.

### CROSS REFERENCE TO RELATED APPLICATIONS

This current application is a continuation of U.S. patent application Ser. No. 16/796,851, filed Feb. 20, 2020, entitled “Inducible Production-Phase Promoters for Coordinated Heterologous Expression in Yeast” to Harvey et al., which is a continuation of U.S. patent application Ser. No. 15/469,452, filed Mar. 24, 2017, entitled “Inducible Production-Phase Promoters for Coordinated Heterologous Expression in Yeast” to Harvey et al., which claims priority under 35 U.S.C. 119(e) to U.S. Provisional Application Ser. No. 62/313,108, filed Mar. 24, 2016, the disclosures of which are each incorporated herein by reference in its entirety.

### STATEMENT REGARDING FEDERALLY SPONSORED RESEARCH OR DEVELOPMENT

This invention was made with Government support under contract GM110706 awarded by the National Institutes of Health. The Government has certain rights in the invention.

### REFERENCE TO A SEQUENCE LISTING SUBMITTED ELECTRONICALLY VIA EFS-WEB

The instant application contains a Sequence Listing which has been filed electronically in ASCII format and is hereby incorporated by reference in its entirety. Said ASCII copy, created on Jun. 3, 2020, is named “05041 CONseqlist_ST25.txt” and is 104 bytes in size.

### FIELD OF THE INVENTION

The invention is generally directed to systems and constructs for heterologous expression in yeast, and more specifically to a set of inducible promoters that can be combined for coordinated expression of multiple genes and methods related to their construction and use.

### BACKGROUND

Saccharomyces (S.) is a genus of fungi composed of different yeast species. The genus can be divided into two further subgenera S. sensu stricto and S. sensu lato. The former have relatively similar characteristics, including the ability to interbreed, exhibiting uniform karyotype of sixteen chromosomes, and their use in the fermentation industry. The later are more diverse and heterogeneous. Of particular importance is the S. cerevisiae species within the S. sensu stricto subgenus, which is a popular model organism used for genetic research.

The yeast S. cerevisiae is a powerful host for the heterologous expression of biosynthetic systems, including production of biofuels, commodity chemicals, and small molecule drugs. The yeast's genetic tractability, ease of culture at both small and large scale, and a suite of well-characterized genetic tools make it a desirable system for heterologous expression. Occasionally, production systems require coordinated expression of two or more heterologous genes. Coordinated expression systems in bacteria (e.g., E. coli) has long exploited the operon structure of bacterial gene clusters (e.g., lac operon), allowing a single promoter to control the expression of multiple genes. The construction of synthetic operons therefore allows a single inducible promoter to control the timing and strength of expression of an entire synthetic system. In yeast, many heterologous-expression systems do not rely on the operon system, but instead rely on a one-promoter, one-gene paradigm. Accordingly, multi-gene heterologous expression in most yeast strains is performed using multiple expression cassettes with a well-characterized promoter and terminator, each on a single expression vector (e.g., plasmid DNA) (See D. Mumberg, R. Muller, and M. Funk Gene 156:119-22, 1995, which is incorporated herein by reference). With traditional restriction-ligation cloning, it is also possible to recycle a promoter on a single plasmid by the serial cloning of multiple genes (M. C. Tang, et al., J Am Chem Soc 137:13724-27, 1995).

### SUMMARY OF THE INVENTION

Many embodiments of the invention are directed to a DNA molecule composition comprising at least one exogenous DNA vector comprising at least two different production-phase promoters; wherein the two production-phase promoters are each capable of repressing heterologous expression of an exogenous gene in a Saccharomyces cerevisiae cell when the S. cerevisiae cell predominantly exhibits anaerobic energy metabolism; and wherein the two production-phase promoters are each also capable of inducing heterologous expression of the exogenous gene in the S. cerevisiae cell when the S. cerevisiae cell predominantly exhibits aerobic energy metabolism.

In further embodiments the at least one exogenous DNA vector further comprising a heterologous gene; wherein the heterologous gene Sequence is derived from a species other than S. cerevisiae; and wherein the heterologous gene is situated proximately downstream of one of the two production promoters such that the heterologous gene expression can be repressed and induced by the production promoter that is proximately upstream from the heterologous gene.

In more embodiments, the anaerobic energy metabolism is defined by the catabolism of a fermentable carbon source.

In further more embodiments, the fermentable carbon source is glucose or dextrose.

In even further more embodiments, the aerobic energy metabolism is defined by the catabolism of a nonfermentable carbon source.

In even further more embodiments, the nonfermentable carbon source is ethanol or glycerol.

In even further more embodiments, the DNA molecule compositions further comprise a S. cerevisiae cell, wherein the exogenous DNA vector exists within the S. cerevisiae cell.

In even further more embodiments, at least one of the at least two production phase promoters comprises a sequence of an endogenous production-phase promoter of S. cerevisiae.

In even further more embodiments, the production-phase promoter is substantially similar to a sequence selected from the group consisting of the S. cerevisiae ADH2 promoter (Seq. ID No. 1), S. cerevisiae PCK1 promoter (Seq. ID No. 2), the S. cerevisiae MLS1 promoter (Seq. ID No. 3), the S. cerevisiae ICL1 promoter (Seq. ID No. 4), the S. cerevisiae YLR307C-A promoter (Seq. ID No. 5), the S. cerevisiae YGR067C promoter (Seq. ID No. 6), the S. cerevisiae IDP2 promoter (Seq. ID No. 7), the S. cerevisiae ADY2 promoter (Seq. ID No. 8), the S. cerevisiae GAC1 promoter (Seq. ID No. 9), the S. cerevisiae ECM13 promoter (Seq. ID No. 10), the S. cerevisiae FAT3 promoter (Seq. ID No. 11), the S. cerevisiae PULT1 promoter (Seq. ID No. 12), the S. cerevisiae NQM1 promoter (Seq. ID No. 13), the S. cerevisiae SFC1 promoter (Seq. ID No. 14), the S. cerevisiae JEN1 promoter (Seq. ID No. 15), the S. cerevisiae SIP18 promoter (Seq. ID No. 16), the S. cerevisiae AT02 promoter (Seq. ID No. 17), the S. cerevisiae YIG1 promoter (Seq. ID No. 18), and the S. cerevisiae FBP1 promoter (Seq. ID No. 19).

In even further more embodiments, at least one of the at least two production phase promoters comprises a Sequence of an exogenous production-phase promoter of S. cerevisiae.

In even further more embodiments, the production-phase promoter is substantially similar to a sequence selected from the group consisting of the S. paradoxus ADH2 promoter (Seq. ID No. 36), the S. kudriavzevii ADH2 promoter (Seq. ID No. 37), S. bayanus ADH2 promoter (Seq. ID No.38), S. paradoxus PCK1 promoter (Seq. ID No. 41), the S. kudriavzevii PCK1 promoter (Seq. ID No. 42), S. bayanus PCK1 promoter (Seq. ID No. 43), S. paradoxus MLS1 promoter (Seq. ID No. 44), the S. kudriavzevii MLS1 promoter (Seq. ID No. 45), S. bayanus MLS1 promoter (Seq. ID No. 46), S. paradoxus ICL1 promoter (Seq. ID No. 47), the S. kudriavzevii CL1 promoter (Seq. ID No. 48), and S. bayanus ICL1 promoter (Seq. ID No. 49).

Many embodiments are directed to at least one exogenous DNA vector comprising a production-phase promoter, wherein the production-phase promoter is substantially similar to a sequence selected from the group consisting of the S. cerevisiae PCK1 promoter (Seq. ID No. 2), the S. cerevisiae MLS1 promoter (Seq. ID No. 3), the S. cerevisiae ICL1 promoter (Seq. ID No. 4), the S. cerevisiae YLR307C-A promoter (Seq. ID No. 5), the S. cerevisiae YGR067C promoter (Seq. ID No. 6), the S. cerevisiae IDP2 promoter (Seq. ID No. 7), the S. cerevisiae ADY2 promoter (Seq. ID No. 8), the S. cerevisiae GAC1 promoter (Seq. ID No. 9), the S. cerevisiae ECM13 promoter (Seq. ID No. 10), the S. cerevisiae FAT3 promoter (Seq. ID No. 11), the S. cerevisiae PULT1 promoter (Seq. ID No. 12), the S. cerevisiae NQM1 promoter (Seq. ID No. 13), the S. cerevisiae SFC1 promoter (Seq. ID No. 14), the S. cerevisiae JEN1 promoter (Seq. ID No. 15), the S. cerevisiae SIP18 promoter (Seq. ID No. 16), the S. cerevisiae AT02 promoter (Seq. ID No. 17), the S. cerevisiae YIG1 promoter (Seq. ID No. 18), the S. cerevisiae FBP1 promoter (Seq. ID No. 19), the S. paradoxus ADH2 promoter (Seq. ID No. 36), the S. kudriavzevii ADH2 promoter (Seq. ID No. 37), S. bayanus ADH2 promoter (Seq. ID No.38), S. paradoxus PCK1 promoter (Seq. ID No. 41), the S. kudriavzevii PCK1 promoter (Seq. ID No. 42), S. bayanus PCK1 promoter (Seq. ID No. 43), S. paradoxus MLS1 promoter (Seq. ID No. 44), the S. kudriavzevii MLS1 promoter (Seq. ID No. 45), S. bayanus MLS1 promoter (Seq. ID No. 46), S. paradoxus ICL1 promoter (Seq. ID No. 47), the S. kudriavzevii ICL1 promoter (Seq. ID No. 48), and S. bayanus ICL1 promoter (Seq. ID No. 49).

In further embodiments, the selected production-phase promoter is substantially similar to the S. cerevisiae PCK1 promoter sequence (Seq. ID No. 2).

In more embodiments, the selected production-phase promoter is substantially similar to the S. cerevisiae MLS1 promoter sequence (Seq. ID No. 3).

In further more embodiments, the selected production-phase promoter is substantially similar to the S. cerevisiae ICL1 promoter sequence (Seq. ID No. 4).

In even further more embodiments, the selected production-phase promoter is substantially similar to a sequence selected from the group consisting of the S. paradoxus ADH2 promoter (Seq. ID No. 36), the S. kudriavzevii ADH2 promoter (Seq. ID No. 37), and S. bayanus ADH2 promoter (Seq. ID No. 38).

In even further more embodiments, the selected the production-phase promoter is substantially similar to a sequence selected from the group consisting of S. paradoxus PCK1 promoter (Seq. ID No. 41), the S. kudriavzevii PCK1 promoter (Seq. ID No. 42), S. bayanus PCK1 promoter (Seq. ID No. 43), S. paradoxus MLS1 promoter (Seq. ID No. 44), the S. kudriavzevii MLS1 promoter (Seq. ID No. 45), S. bayanus MLS1 promoter (Seq. ID No. 46), S. paradoxus ICL1 promoter (Seq. ID No. 47), the S. kudriavzevii CL1 promoter (Seq. ID No. 48), and S. bayanus ICL1 promoter (Seq. ID No. 49).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description will be more fully understood with reference to the following figures and data graphs, which are presented as exemplary embodiments of the invention and should not be construed as a complete recitation of the scope of the invention.

FIG. 1A illustrates a yeast phase chart displaying yeast cell concentration in relation to time to provide reference for various embodiments of the invention.

FIG. 1B illustrates a yeast phase chart displaying glucose concentration in relation to time to provide reference for various embodiments of the invention.

FIG. 1C illustrates a yeast phase chart displaying ethanol or glycerol concentration in relation to time to provide reference for various embodiments of the invention.

FIG. 2A illustrates a DNA vector having a production-phase promoter in accordance with an embodiment of the invention.

FIG. 2B illustrates a DNA vector having multiple production-phase promoters in accordance with an embodiment of the invention.

FIG. 3A illustrates a DNA expression vector having a production-phase promoter within an expression cassette in accordance with an embodiment of the invention.

FIG. 3B illustrates a DNA expression vector having multiple production-phase promoters, each within an expression cassette in accordance with an embodiment of the invention.

FIG. 4 illustrates a method to construct and utilize production-phase promoter DNA vectors in accordance with various embodiments of the invention.

FIG. 5 is a heat map graphic generated in accordance with various embodiments of the invention with data of expression of enhanced-Green Fluorescent Protein driven by various S. cerevisiae promoters.

FIG. 6 is a data graph of enhanced-Green Fluorescent Protein expression driven by various S. cerevisiae promoters, generated in accordance with various embodiments of the invention.

FIG. 7 illustrates fluorescence intensity of enhanced-Green Fluorescent Protein driven by various promoters, generated in accordance with various embodiments of the invention.

FIG. 8 illustrates a phylogenetic tree of Saccharomyces sensu stricto subgenus to provide reference for various embodiments of the invention.

FIG. 9 illustrates a multiple sequence alignment of various Saccharomyces sensu stricto species' upstream activating sequences in ADH2 promoters to provide reference for various embodiments of the invention.

FIG. 10 illustrates homology between various Saccharomyces sensu stricto species' ADH2 promoters to provide reference for various embodiments of the invention.

FIG. 11 is a heat map graphic generated in accordance with various embodiments of the invention with data of expression of enhanced-Green Fluorescent Protein driven by various S. sensu stricto ADH2 promoters.

FIG. 12 is a data graph of enhanced-Green Fluorescent Protein expression driven by various S. sensu stricto ADH2 promoters, generated in accordance with various embodiments of the invention.

FIG. 13 illustrates four multi-gene expression vector constructs, each to generate a product compound, in accordance with an embodiment of the invention.

FIG. 14 illustrates a biosynthetic process that produces the compound emindole SB via a fungal four-gene cluster to provide reference for various embodiments of the invention.

FIG. 15 is a data graph of the production results of two product compounds generated in accordance of an embodiment of the invention.

FIG. 16 illustrates two plasmid vector constructs in accordance with an embodiment of the invention.

### BRIEF DESCRIPTION OF THE SEQUENCE LISTING

The current disclosure incorporates a sequence listing in accordance with the WIPO Standard ST.25. The Sequence listing embodies sixty-six nucleic acid sequences (Seq ID Nos. 1-66), which are referenced in Table 3 and throughout the specification.

### DETAILED DESCRIPTION

Turning now to the drawings and data, embodiments of the invention are generally directed to systems and constructs of heterologous expression during the production phase of yeast. In many of these embodiments, the expression system involves coordinated expression of multiple heterologous genes. More embodiments are directed to production-phase promoter systems having promoters that are inducible upon an event in the yeast's growth or by the nutrients and supplements provided to the yeast. Specifically, a number of embodiments are directed to the promoters that are capable of being repressed in the presence of glucose and/or dextrose. In more embodiments, the promoters are capable of being induced in the presence of glycerol and/or ethanol. In additional embodiments, at least one production-phase promoter exists within an exogenous DNA vector, such as (but not limited to), for example, a shuttle vector, cloning vector, and/or expression vector. Embodiments are also directed to the use of expression vectors for the expression of heterologous genes in a yeast expression system.

Controlled gene expression is desirable in heterologous expression systems. For example, it would be desirable to express heterologous genes for production during a longer stable phase. Accordingly, decoupling the anaerobic growth and aerobic production phases of a culture allows the yeast to grow to high density prior to introducing the metabolic stress of expressing unnaturally high amounts of heterologous protein. In accordance with many embodiments, he anaerobic growth phase is defined by the yeast culture's energy metabolism in which the yeast cells predominantly catabolize fermentable carbon sources (e.g., glucose and/or dextrose), and a high growth rate (i.e., short doubling-time). In contrast, and in accordance with several embodiments, the aerobic production phase is defined by the yeast culture's energy metabolism in which the yeast cells predominantly catabolize nonfermentable carbon sources (e.g., ethanol and/or glycerol), and a steady growth rate (i.e., long doubling-time). Accordingly, each yeast cell's energy metabolism is binary and dependent on the local concentration of the carbon source.

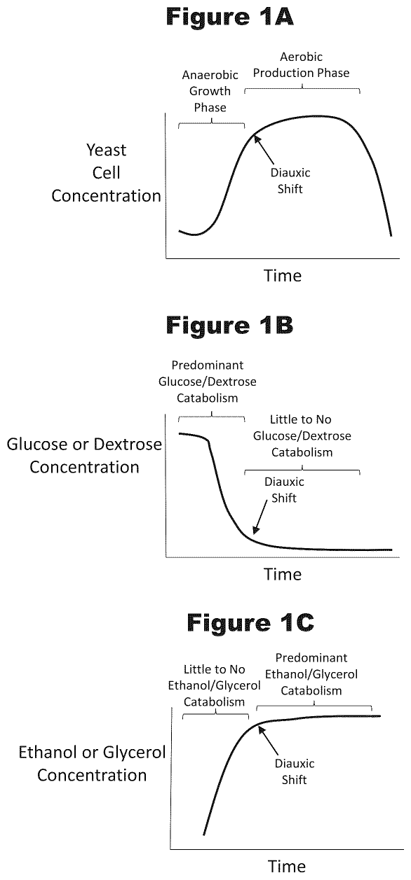

FIG. 1A depicts the phases of a yeast culture when provided a fermentable sugar, such as glucose or dextrose sugar, at a concentration of around 2-4% as its main carbon source. Initially, a yeast culture will predominantly catabolize the fermentable sugar, which correlates with an exponential growth with very high doubling rates. The growth phase typically lasts approximately 4-10 hours. During this phase, the catabolism of the fermentable sources results in the production of ethanol and glycerol.

Once glucose becomes scarce, the growth of a yeast culture passes a diauxic shift and begins to predominantly catabolize nonfermentable carbon sources (e.g., ethanol and/or glycerol) (FIG. 1B). The predominant catabolism of nonfermentable carbon source correlates with a longer and more stable production phase that can last for several days, or even weeks in an industrial-like setting (FIG. 1A). During the production phase, yeast cultures reach and maintain a high concentration, but have a much lower doubling time (FIG. 1A). Due to the decrease in doubling rate, yeast cultures no longer expend a great amount of energy and resources on rapid growth and thus can reallocate that energy and those resources to other biological activities, including heterologous expression. Accordingly, it is hypothesized that limiting the transcription of heterologous genes to the production phase would allow a yeast culture to reach a high, healthy confluency that would in turn allow better heterologous protein expression and biosynthetic production.

In yeast, transcriptional regulation can be achieved in several ways, including inducement by chemical substrates (e.g., copper or methionine), the tetON/OFF system, and promoters engineered to bind unnatural hybrid transcription factors. Perhaps the most commonly employed inducible promoters are the promoters controlled by the endogenous GAL4 transcription factor. GAL4 promoters are strongly repressed in glucose, and upon switching to galactose as a carbon source, strong induction of transcription is observed (M. Johnston and R. W. Davis, Mol. Cell Biol. 4:1440-48, 1984, the disclosure of which is incorporated herein by reference). While this system leads to high-level transcription, only four galactose-responsive promoters are known, and galactose is both a more expensive and a less efficient carbon source as compared to glucose (S. Ostergaard, et al., Biotechnol. Bioeng. 68:252-59, 2000, the disclosure of which is incorporated herein by reference).

Other carbon-source dependent promoters have also been used for heterologous gene expression. The S. cerevisiae ADH2 gene exhibits significant derepression upon depletion of glucose as well as strong induction by either glycerol or ethanol (K. M. Lee & N. A. DeSilva Yeast. 22:431-40, 2005, the disclosure of which is incorporated herein by reference). Once induced, genes driven by the ADH2 promoter (pADH2) display expression levels equivalent to those driven by highly expressed constitutive counterparts. This induction profile was found to work in heterologous expression studies, as the system auto-induces upon glucose depletion in the late stages of fermentative growth after cells have undergone diauxic shift. The ADH2 promoter has been used extensively for yeast heterologous expression studies, resulting in high-level expression of several heterologous biosynthetic proteins (For example, see C. D. Reeves, et al., Appl. Environ. Microbiol. 74:5121-29, 2008, the disclosure of which is incorporated herein by reference).

As shown in FIG. 1C, the concentration of ethanol and glycerol increases as glucose and dextrose sugar decreases, due to anaerobic glycolysis (i.e., breaking down the fermentable sugar) and subsequent fermentation (i.e., converting the broken-down glucose into alcohol) and glycerol biosynthesis (i.e., converting the broken-down glucose into glycerol). Upon fermentable sugar depletion, yeast cultures undergo a diauxic shift and begin to use ethanol and glycerol as a carbon source instead of glucose. A diauxic shift, as understood in the art, is defined as a point in time when an organism switches consumption of one source for energy, to another source. This shift requires significant changes to a yeast culture's gene-expression pattern. Accordingly, it is hypothesized that higher concentrations of ethanol, (i.e., ˜2-4%) and or glycerol (i.e., ˜2%) could be used to stimulate promoters that either directly or indirectly respond to these concentrations (See FIGS. 1A and 1C).

Various embodiments of the invention are based on the discovery of inducible promoters that can be used for the coordinated expression of multiple genes (e.g., gene cluster pathway) in Saccharomyces yeast. Described below are sets of inducible promoters from S. cerevisiae and related species that are inactive during anaerobic growth, activating transcription only after a diauxic shift when glucose is near-depleted and the yeast cells are respiring (i.e., the production phase). As portrayed in various embodiments, various production-phase promoters are auto-inducing and allow automatic decoupling of the growth and production phases of a culture and thus initiate heterologous expression without the need for exogenous inducers. It should be noted, however, that many embodiments of the invention include production-phase promoters that are also inducible in the presence of nonfermentable carbon-sources (e.g., ethanol and/or glycerol) supplied to the yeast. As such, multiple embodiments employ recombinant production-phase promoters that act much like constitutive promoters when the host yeast cultures are constantly maintained in ethanol- and/or glycerol-containing media.

Once activated, the strength of various production-phase promoters can vary as much as 50-fold in accordance with numerous embodiments of the invention. The strongest production-phase promoters stimulate heterologous expression greater than that observed from strong constitutive promoters. The production-phase promoters could be employed in many different applications in which high expression of multiple genes is beneficial. Accordingly, the promoters can be used, for example, in multiple subunit protein production or for the production of biosynthetic compounds that are produced by multiple proteins within a pathway. Discussed in an exemplary embodiment below, embodiments of the invention are used to express multiple proteins involved in production of indole diterpene compound product. When compared to constitutive promoters, the production-phase promoters produced greater than a 2-fold increase in titer of the exemplary diterpene natural products. In other exemplary embodiments, it was found that the production-phase promoter system outperformed constitutive promoters by over 80-fold. Thus, these promoters can enable heterologous expression of biosynthetic systems in yeast.

The practice of several embodiments of the present invention will employ, unless otherwise indicated, conventional methods of chemistry, biochemistry, and molecular biology and recombinant DNA techniques within the skill of the art. Such techniques are explained fully in the literature. See, e.g., A. L. Lehninger, Biochemistry (Worth Publishers, Inc., 30 current addition); Sambrook, et al., Molecular Cloning: A Laboratory Manual (3^{rd} Edition, 2001); Methods In Enzymology (S. Colowick and N. Kaplan eds., Academic Press, Inc.).

Inducible Production-Phase Promoters for Heterologous Expression in Yeast

In accordance with several embodiments of the invention, inducible production-phase promoters can be constructed into exogenous expression vectors for production of at least one protein in Saccharomyces yeast. In many embodiments, the constructed expression vectors have multiple inducible production-phase promoters in order to express multiple heterologous genes.

Several embodiments are directed to production-phase promoters and DNA vectors incorporating these promoters. Promoters, in general, are defined as a noncoding portion of DNA sequence situated proximately upstream of a gene to regulate and promote its expression. Typically, in S. cerevisiae and similar species, the promoter of a gene can be found within 500-bp upstream of a gene's translation start codon.

In accordance with several embodiments, production-phase promoters have two defining characteristics. First, production-phase promoters are capable of repressing heterologous expression of a gene in S. cerevisiae and similar species when the yeast is exhibiting anaerobic energy metabolism. As described previously, yeast exhibit anaerobic metabolism in the presence of a nontrivial concentration of fermentable carbon sources such as, for example, glucose or dextrose. In addition, production-phase promoters are also capable of inducing heterologous expression of a gene in S. cerevisiae and similar species when the yeast is exhibiting aerobic energy metabolism. As described previously, yeast exhibit aerobic metabolism when fermentable carbon sources are near depleted and the yeast cells switch to a catabolism of nonfermentable carbon sources such as glycerol or ethanol. These characteristics correspond to the phase charts in FIGS. 1A-1C. Tables 1 and 2 provide several examples of production-phase promoters in accordance with several embodiments. Table 3 provides sequences that correspond with the promoters and the incorporated sequence listing.

The production-phase promoters can be characterized based on their level of transgene expression relative to each other and to constitutive promoters. As described in an exemplary embodiment below, it was found that the sequence of endogenous promoters of the S. cerevisiae genes ADH2, PLK1, MLS1, and ICL1 exhibited high-level expression and thus can be characterized as strong production-phase promoters (Table 1). Sequences of the endogenous promoters of the S. cerevisiae genes YLR37C-A, ORF-YGRO67C IDP2, ADY2, CAC1, ESM13, and FAT3 exhibited mid-level expression and thus can be characterized as semi-strong production phase promoters (Table 1). In addition, sequences of the endogenous promoters of the S. cerevisiae genes PUT1, NQM1, SFD1, JEN1, 2IP18, AT2, YIG1, and FBP1 exhibited low-level expression and thus can be characterized as weak production-phase promoters (Table 1).

TABLE 1
Production-Phase Promoters Expression Phenotype

| Gene | Systematic | Expression | Sequence |
|---|---|---|---|
| Name | Name | Phenotype | ID Number |
| ADH2 | YMR303C | Strong | 1 |
| PCK1 | YKR097W | Strong | 2 |
| MLS1 | YNL117W | Strong | 3 |
| ICL1 | YER065C | Strong | 4 |
| YLR307C-A | YLR307C-A | Semi-Strong | 5 |
| YGR067C | YGR067C | Semi-Strong | 6 |
| IDP2 | YLR174W | Semi-Strong | 7 |
| ADY2 | YCR010C | Semi-Strong | 8 |
| GAC1 | YOR178C | Semi-Strong | 9 |
| ECM13 | YBL043W | Semi-Strong | 10 |
| FAT3 | YKL187C | Semi-Strong | 11 |
| PUT1 | YLR142W | Weak | 12 |
| NQM1 | YGR043C | Weak | 13 |
| SFC1 | YJR095W | Weak | 14 |
| JEN1 | YKL217W | Weak | 15 |
| SIP18 | YMR175W | Weak | 16 |
| ATO2 | YNR002C | Weak | 17 |
| YIG1 | YPL201C | Weak | 18 |
| FBP1 | YLR377C | Weak | 19 |

The closely related S. sensu stricto species have similar genetics and growth characteristics. Accordingly, the phase charts provided in FIGS. 1A-1C apply generally to S. sensu stricto species. Table 2 provides a list of strong production-phase exogenous promoters of similarly related species in accordance with numerous embodiments of the invention.

TABLE 2
Strong Production-Phase Promoters of S. sensu stricto species

| | Gene | Sequence |
|---|---|---|
| Species | Name | ID Number |
| S. paradoxus | ADH2 | 36 |
| S. kudriavzevii | ADH2 | 37 |
| S. bayanus | ADH2 | 38 |
| S. paradoxus | PCK1 | 41 |
| S. kudriavzevii | PCK1 | 42 |
| S. bayanus | PCK1 | 43 |
| S. paradoxus | MLS1 | 44 |
| S. kudriavzevii | MLS1 | 45 |
| S. bayanus | MLS1 | 46 |
| S. paradoxus | ICL1 | 47 |
| S. kudriavzevii | ICL1 | 48 |
| S. bayanus | ICL1 | 49 |

It should be noted that substantially similar sequences to the production-promoter sequences are expected to regulate heterologous expression in S. cerevisiae and achieve similar results. Accordingly, a substantially similar sequence of a production-phase promoter, in accordance with numerous embodiments, is any sequence with a high homology such that when regulating heterologous expression in S. cerevisiae that it achieves substantially similar results. For example, in an exemplary embodiment below, it was found that the ADH2 promoter of S. bayanus is only 61% homologous, yet achieved strong heterologous expression in S. cerevisiae, similar to the endogenous ADH2 promoter.

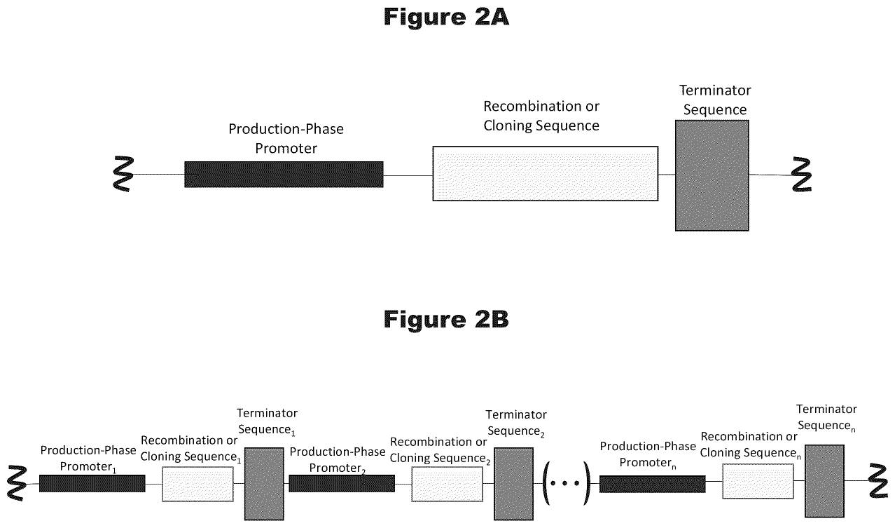

In FIG. 2A, an exemplary schematic of a section of an exogenous DNA vector (e.g., cloning vector, expression vector, and/or shuttle vector) having a production-phase promoter sequence embedded within. A vector is capable of transferring nucleic acid sequences to target cells (e.g., yeast). Typical DNA vectors include, but are not limited to, plasmid or viral constructs. DNA vectors are also meant to include a kit of various linear DNA fragments that are to be recombined to form a plasmid or other functional construct, as is common in yeast homologous recombination methods (See e.g., Z. Shao, H. Zhao & H. Zhao, 2009, Nucleic Acids Research 37:e16, 2009, the disclosure of which is incorporated herein by reference). Often, embodiments of cloning vectors will incorporate other sequences in addition to the production-phase promoter. As depicted in FIG. 2A, the exemplary cloning vector has a terminator sequence and cloning/recombination sequence in addition to the production-phase promoter, each of which can assist with expression vector construction. Furthermore, other sequences necessary for growth and amplification can be incorporated into the promoter vector. Embodiments of these sequences may include, for example, at least one appropriate origin of replication, at least one selectable marker, and/or at least one auxotrophic marker. It should be noted, however, that various embodiments of the invention are not required to contain cloning, terminator, or either sequences. For example, embodiments of a typical shuttle vector may only contain the production-phase promoter sequence along with the necessary sequences for amplification in a biological system.

For purposes of this application, an exogenous DNA vector is any DNA vector that was constructed, at least in part, exogenously. Accordingly, DNA vectors that are assembled using the yeast's own cell machinery (e.g., yeast homologous recombination) would still be considered exogenous if any of the DNA molecules transduced within yeast for recombination contain exogenous sequence or were produced by a non-host methodology, such as, for example, chemical synthesis, PCR amplification, or bacterial amplification.

As shown in FIG. 2B, various embodiments of the invention are directed to DNA vectors having multiple production-phase promoters. In these various embodiments, multiple different production-phase promoters are incorporated, preferably each having a unique sequence and derived from a different gene and/or S. sensu stricto species. Having unique promoter sequences can prevent complications that can arise during product production in yeast, such as, for example, unwanted DNA recombination at sites similar to the promoter sequences that render the DNA vector constructs undesirable. In many embodiments, the DNA vector has at least two production-phase promoters and up to a number that still yields the vector useful. As the size of the DNA vector increases, the utility may decrease, as larger vectors may become unwieldly for the intended organism to handle. For example, plasmids for amplification in E. coli are often somewhere between 2,000 and 10,000 base pairs (bp) but can handle up to 20,000 bp or so. Likewise, plasmids for amplification and growth in yeast can vary from approximately 10,000 to 30,000 bp. Viral vectors, on the other hand, often have a limited construct size and thus may require a more precise vector size. Thus, depending on vector and intended use, the number of production-phase promoters within a DNA vector will vary.

Although FIG. 2B depicts recombination sites, cloning sites, and terminator sequences, it should be noted that these sequences may or may not be included in various embodiments of DNA vectors having multiple production-phase promoters. The incorporation of these sequences or other various sequence is often dependent on the purpose of the DNA vector. For example, cloning vectors may not include a terminator sequence if that sequence is to be incorporated into an expression construct at another stage of assembly.

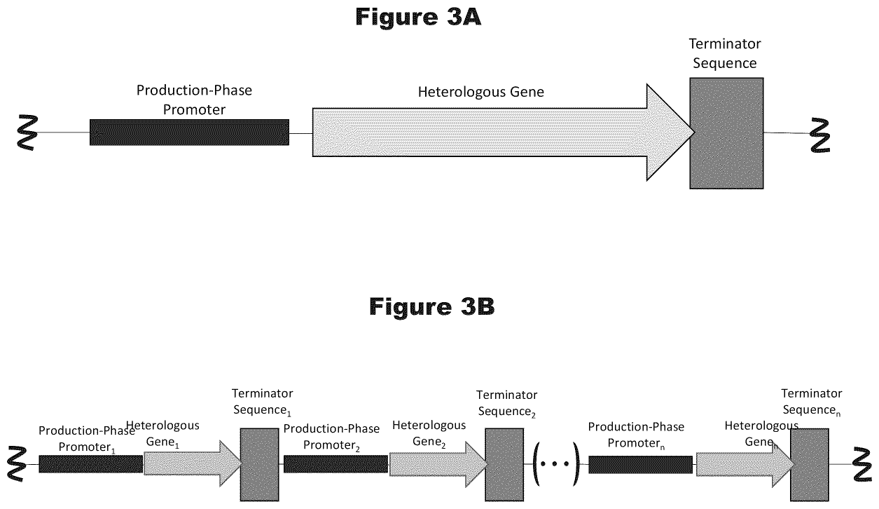

FIG. 3A depicts an exemplary heterologous expression vector having a production-phase promoter for expression in yeast, in accordance with various embodiments of the invention. Expression constructs contain an expression cassette that minimally has a promoter, a heterologous gene, and a terminator sequence in order to produce an RNA molecule in an appropriate host. Expression cassette in accordance with numerous embodiments will have a production-phase promoter situated proximately upstream of a heterologous gene of which the promoter is to regulate expression. It should be understood, that the precise location of the production-phase promoter upstream of the heterologous gene may vary, but the promoter must be within a certain proximity to adequately function.

In many embodiments of the invention, a heterologous gene is any gene driven by a production-phase promoter, wherein the heterologous gene is different than the endogenous gene that the promoter regulates within its endogenous genome. Accordingly, a S. cerevisiae production-phase promoter could regulate another S. cerevisiae gene provided that the gene to be regulated is not the gene endogenously regulated. For example, the S. cerevisiae ADH2 promoter should not regulate the S. cerevisiae ADH2 gene; however, the S. cerevisiae ADH2 promoter can regulate any other S. cerevisiae gene or the ADH2 gene from any other species. Often, in accordance with many embodiments, the heterologous gene is from a different species than the species from which the production-promoter sequence was obtained.

Although not depicted, various embodiments of expression cassettes may include other sequences, such as, for example, intron sequences, Kozak-like sequences, and/or protein tag sequences (e.g., 6x-His) that may or may not improve expression, production, and/or purification. In yeast, various embodiments of expression vectors will also minimally have a yeast origin of replication (e.g., 2-micron) and an auxotrophic marker (e.g., URA3) in addition to the expression cassette. Other nonessential sequences may also be included, such as, for example, bacterial origins of replication and/or bacterial selection markers that would render the expression capable of amplification in a bacterial host in addition to a yeast host. Accordingly, various embodiments of expression vectors would include the essential sequences for heterologous expression in yeast and other various embodiments would include additional nonessential sequences.

In accordance with various embodiments, a DNA vector having a production-phase promoter expression cassette can be transformed into a yeast cell. Or alternatively, and in accordance with numerous embodiments, a DNA vector having a production-phase promoter expression cassette can be assembled within yeast using homologous recombination techniques. Once existing within a yeast cell, the production-phase promoter can regulate the expression of a heterologous gene in accordance with the yeast cell's energy metabolism. As described previously, and in accordance with many embodiments, production-phase promoters repress heterologous expression when the yeast cell is in an anaerobic energy metabolic state. Alternatively, and in accordance with a number of embodiments, production-phase promoters induce heterologous expression when the yeast cell is in an aerobic energy metabolic state.

Depicted in FIG. 3B are alternative exemplary heterologous expression vectors having multiple production-phase promoters for expression of multiple genes in yeast in accordance with numerous embodiments. In these embodiments, the expression vectors will include at least two expression cassettes, each with a unique promoter, gene, and terminator sequence in order to prevent unwanted recombination. The number of expression cassettes will vary based on vector construct design and application. For heterologous expression in S. cerevisiae, it has been found that plasmid expression vectors of approximately 30,000 bp are still tolerated. Thus, vectors containing up to seven production-phase promoter expression cassettes can be incorporated into an expression vector and have been found to be able to maintain adequate gene expression and protein production. Larger vectors with more expression cassettes may be tolerated.

Although FIG. 3B depicts multiple expression cassettes sequentially in the same orientation 5′ to 3′, it should be understood that the combination of two or more expression cassettes is not limited to sequential linear organization in the same orientation. Expression cassettes in accordance with many embodiments exist within the expression vector in any orientation and in any sequential order. Furthermore, it should be understood that other sequence elements of an expression vector (e.g., auxotrophic marker) may be among and/or between the multiple expression cassettes. Optimal vector design is likely to depend on various factors, such as, for example, optimizing the location of the auxotrophic marker to enable the final expression vector to include each expression cassette to be incorporated.

DNA heterologous expression vectors are a class of DNA vectors, and thus the description of general DNA vectors above also applies to the expression vectors. Accordingly, many embodiments of the expression vectors are formulated into a plasmid vector, a viral vector, or a kit of linear DNA fragments to be recombined into a plasmid by yeast homologous recombination. In several of these embodiments, the end-product vector contains at least one expression cassette having a production-phase promoter. It should be understood, that in addition to the at least one production-phase promoter, some vector embodiments incorporate expression cassettes that include other promoters, such as (but not limited to), constitutive promoters that maintain high expression during the growth and production phases.

The various embodiments of heterologous expression vectors having at least one production-phase promoter can be used in numerous applications. For example, high expression in the production phase can lead to better, prolonged expression, as compared to constitutive promoters. In many applications, the end product is a protein from a single gene or a protein complex of multiple genes to be purified from the culture. For these applications, high, prolonged expression using production-phase promoters can lead to better yields of proteins. Furthermore, when the heterologous protein is toxic to the host yeast cells, the use of production-phase promoters prevents the expression of the toxic protein during growth phase, allowing the yeast to reach a healthy confluency before mass protein production.

The production-phase promoter vectors can also benefit the production of a biosynthetic compound from a gene cluster. Many products derived from various natural species are produced from a cluster of genes with sequential enzymatic activity. For example, the antibiotic emindole SB is produced from a cluster of four genes that is expressed in Aspergillus tubingensis. To reproduce this gene cluster in a yeast production model, a production-promoter vector system with four different expression cassettes could work. This system would allow the yeast to reach a healthy confluency before the energy-draining expression of four heterologous proteins begin, leading to better overall yields of the antibiotic product. In fact, experimental results provided in an exemplary embodiment described below demonstrate that a production-phase promoter vector outperformed a constitutive promoter vector approximately 2-fold to produce the emindole SB product.

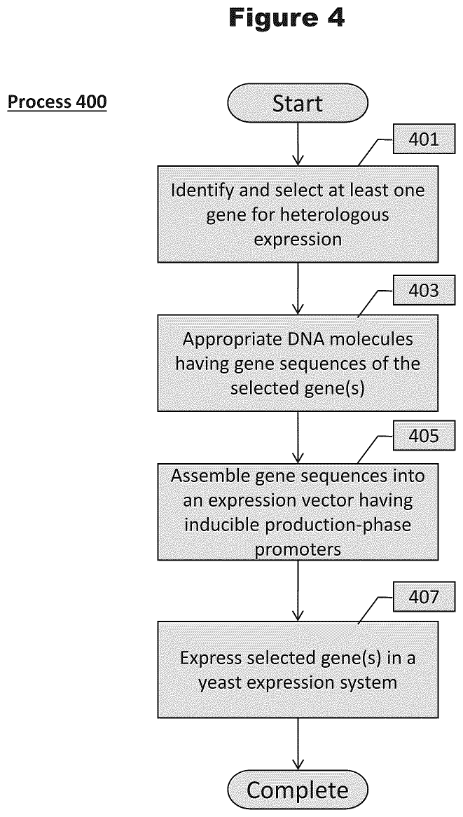

FIG. 4 depicts an exemplary process (Process 400) to implement various embodiments of production-phase promoters. To begin, Process 400 identifies and selects at least one gene for heterologous expression in yeast (401). The choice of gene(s) for expression would depend on the desired outcome. For example, to produce a biosynthetic compound, one would likely select to express all the genes within a biosynthetic gene cluster of a particular organism. Once the gene(s) have been selected, Process 400 then appropriates DNA molecules having the coding sequence of the selected genes (403). As is well known in the art, there are many ways to appropriate DNA molecules, which include chemical synthesis, extraction directly from the biological source, or amplification of a gene by polymerase chain reaction (PCR).

Process 400 then uses the appropriated DNA molecules to assemble these molecules into an expression vector having production-phase promoters (405). There are many ways to assemble DNA expression vectors that are well known in the art, which include popular methodologies such as homologous recombination and restriction digestion with subsequent ligation. After assembly, the resultant expression vectors can be expressed in Saccharomyces yeast to obtain the desired outcome (407).

### Exemplary Embodiments

Biological data supports the systems and constructs of production-phase promoter DNA vectors and applications thereof. Provided below are several examples of incorporating production-phase promoters into DNA vectors. Many of these vectors were used to produce biosynthetic products from multi-gene clusters derived from various fungal species. Compared to a constitutive promoter system, a production-phase promoter system in accordance with various embodiments produced several fold greater product.

Production Phase Promoter Expression Analysis

Because the ADH2 promoter (Seq. ID No. 1) has properties of a production-phase promoter, a panel of promoter sequences was compared to the ADH2 promoter to identify other production-phase promoters. To begin, endogenous S. cerevisiae genes were identified that appeared co-regulated with ADH2 in a previous genome-wide transcription study (Z. Xu. et al., Nature 457:1033-37, 2009, the disclosure of which is incorporated herein by reference). In this study, transcription of yeast genes was quantified during mid-exponential growth in several types of growth media. Of the 5171 ORFs examined, 35 appeared co-regulated with ADH2, with co-regulation defined as a greater than two-fold increase in expression with a non-fermentable carbon source (ethanol in a yeast-peptone-ethanol (YPE) media) as compared to a fermentable carbon source (dextrose in a yeast-peptone-dextrose (YPD) media). Because these data were collected at a single time point and assessed transcription of genes in their native context, their ability to co-regulate heterologous genes in a production-phase promoter system required further validation and characterization.

A detailed characterization of the ability of 34 selected promoters to control expression of heterologous genes was performed. A promoter was defined as the shorter of (a) 500 bp upstream of the start codon, or (b) the entire 5′ intergenic region. Each promoter was cloned upstream of the gene for monomeric enhanced GFP (eGFP) and integrated each of the resulting cassettes in a single copy at the ho locus of individual strains. Control strains were included in which strong constitutive FBA1 and TDH3 promoters were cloned upstream of eGFP in an identical manner. The 35 promoter sequences can be found in Table 3. (Seq. ID Nos. 2-35).

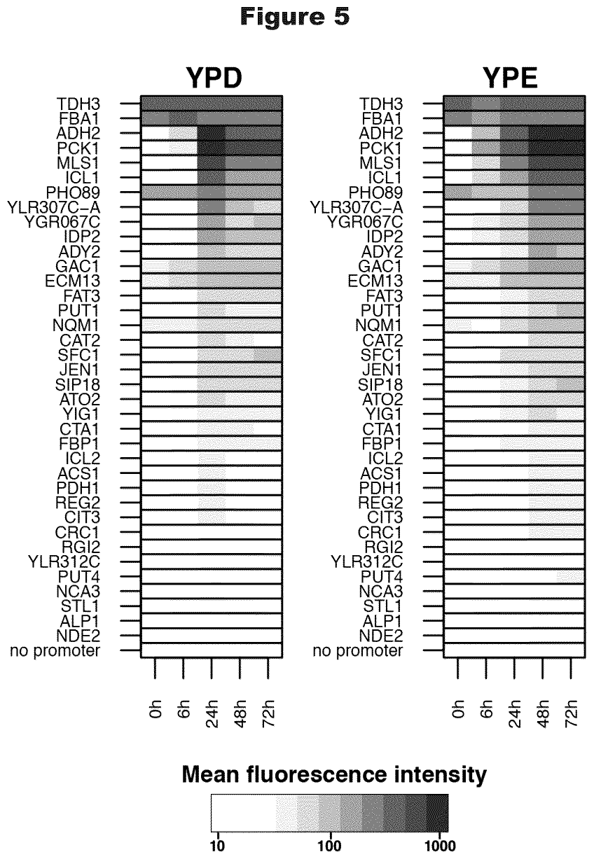

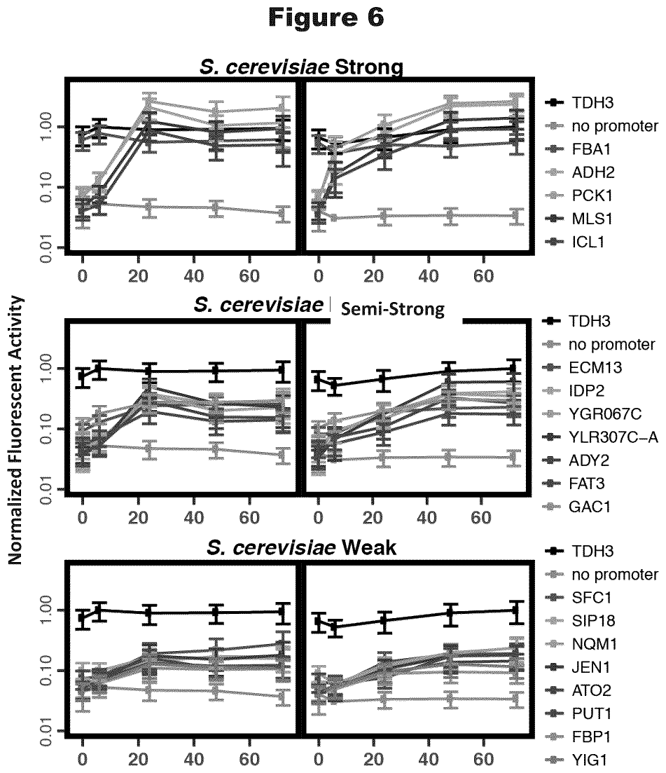

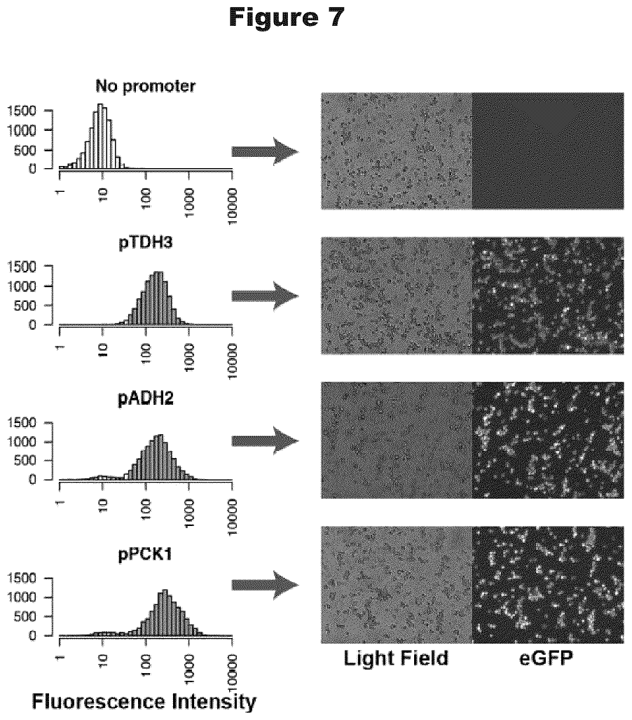

In order to compare the 35 putative production-phase promoters, the expression of eGFP protein was assessed over 72 hours in each strain by flow cytometry in media with both fermentable (YPD) and non-fermentable (YPE) carbon sources (FIGS. 5 and 6). All cultures were started in YPD media and analysis of eGFP expression began when cells were in the midst of exponential fermentative growth (OD600=0.4, 0 hrs). At this point, cells were either left to continue growth in YPD or spun-down and resuspended in YPE. Consistent with previous work, pADH2 was entirely repressed during exponential fermentative growth (0 hrs) unlike the constitutive promoters pTDH3 and pFBA1, which were expressed at near maximum levels regardless of phase. Moderate expression from pADH2 was observed after a further 6 hours in YPD culture or following a growth media switch to YPE. Within 24 hrs, expression reached levels exceeding those observed in the strong constitutive systems. Cytometry histograms and fluorescence microscopy demonstrated that within 48 hours, >95% of all cells with pADH2 and pPCK1 driven expression were fluorescing above background (FIG. 6). Protein expression levels spanned 50-15 fold, with most showing little or no expression until 24 hours into the culture (FIGS. 5 and 6). Transgene expression driven by the PCK1, MLS1, and ICL1 promoters (Seq. ID Nos. 2-4) not only showed the same timing of expression as pADH2, but also expressed at an equivalently high level. The promoters of genes YLR307C-A, YGR067C, IDP2, ADY2, GAC1, ECM13 and FAT3 (Seq. ID Nos. 5-11) displayed semi-strong transgene expression (FIG. 5). In addition, the promoters of genes PUT1, NQM1, SFC1, JEN1, SIP18, ATO2, YIG1, and FBP1 (Seq. ID Nos. 12-19) displayed weak of transgene expression (FIGS. 5 and 6). The promoter PH089 (Seq. ID No. 20) did not exhibit strong repression in during the growth phase (FIG. 5, 0 and 6 hours). The results of the other sequences are also depicted in FIG. 5 (Seq. ID Nos. 22-36). The constitutive promoters pTDH3 and pFBA1 (Seq. ID Nos. 50 and 52) were used as controls (FIGS. 5 and 6).

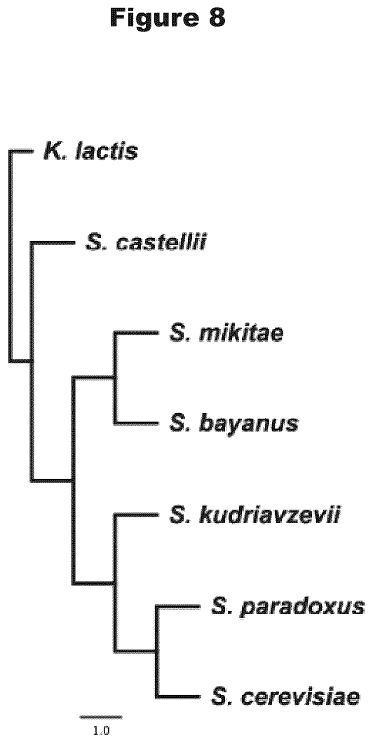

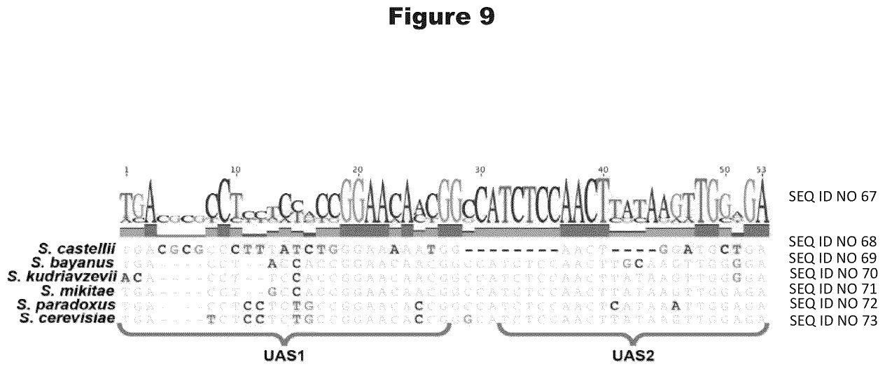

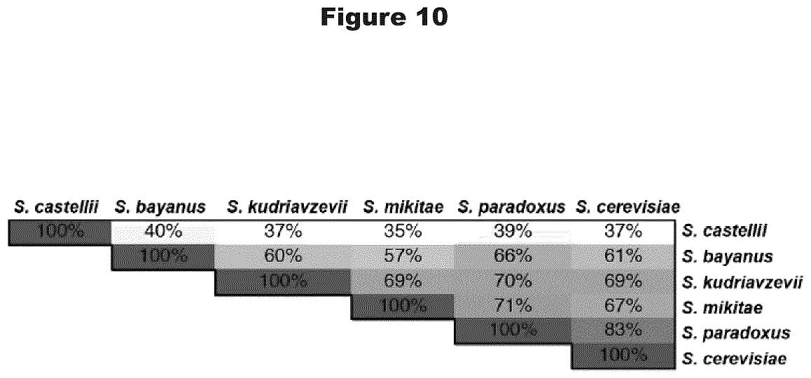

The above analysis identified a large set of co-regulated promoters spanning a wide range of expression levels, three of which were as strong as pADH2. However, a more extensive set of strong production-phase promoters is desirable for assembly of constructs having multi-gene pathways, especially pathways having more than four genes. To identify other production-phase promoter candidates, the genomes of five closely related species within the S. sensu stricto complex were examined (FIG. 8). The promoter region was identified for the closest ADH2 gene homolog in the genomes of Saccharomyces bayanus, Saccharomyces paradoxus, Saccharomyces mikitae, Saccharomyces kudriavzevii, and Saccharomyces castellii. Multiple sequence alignment of the upstream activation sequences (UAS) revealed that nearly all sequences (except that from S. castellii) are highly conserved across this region, suggesting a potential for regulation similar to that of S. cerevisiae ADH2 (FIG. 9, Seq. ID Nos. 36-40). In order to be used for single-step pathway assembly, all promoter sequences must be sufficiently unique to prevent undesired recombination between each other. Therefore, the pairwise identities for each of the Saccharomyces sensu stricto ADH2 promoter pairs were analyzed (FIG. 10). The most similar promoter to the S. cerevisiae ADH2 promoter is that from S. paradoxus, with 83% identity, including a single 40 bp stretch located near the center of the promoter. This homology is significantly less than the 50-100 bp typically used for assembly by yeast homologous recombination, and recombination events between sequences with this level of identity occur at very low frequency, suggesting that these promoters should be compatible with a multi-gene assembly technique utilizing YHR as described above.

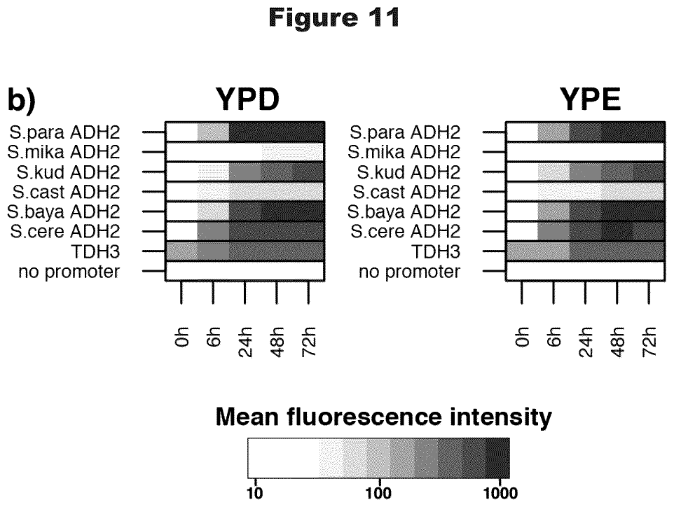

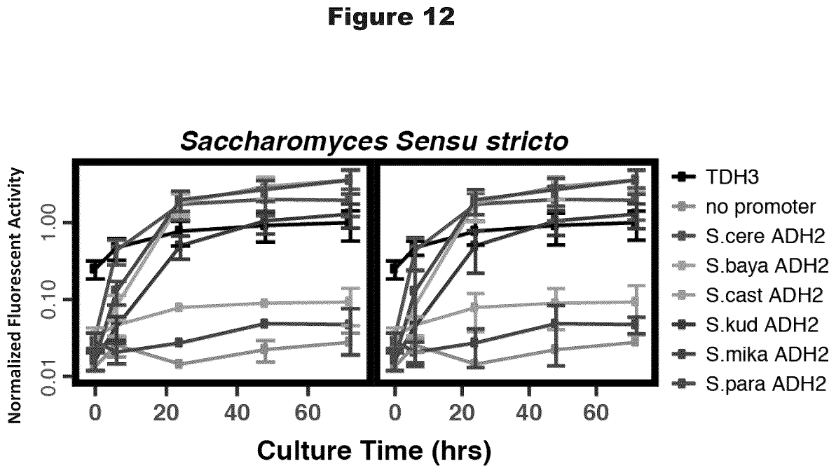

As with the endogenous yeast promoter candidates, these other putative Saccharomyces promoters required detailed characterization of induction profiles. DNA encoding each of these promoter sequences was obtained by commercial synthesis and characterized expression of eGFP from each promoter in the same manner as the endogenous yeast promoters (FIGS. 11 and 12). Of the five Saccharomyces sensu stricto pADH2s tested (Seq. ID Nos. 36-40), the promoters derived from S. paradoxus, S. kudriavzevii, and S. bayanus show timing and strength of expression equivalent to that of S. cerevisiae pADH2. In combination with the endogenous yeast promoters, these three additional Saccharomyces pADH2s expand the number of strong promoters with the desired induction profile.

Expression of Compound Product Pathways Using the Production-Phase Promoter System

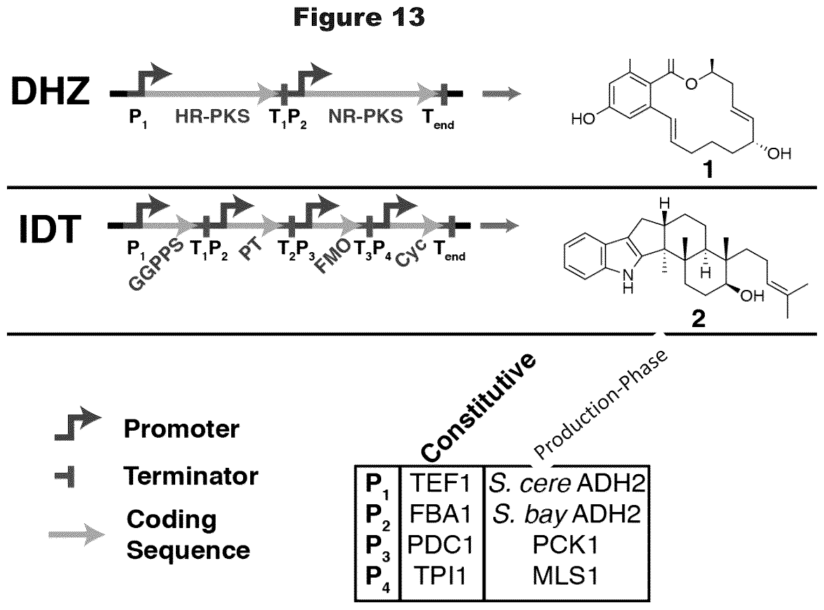

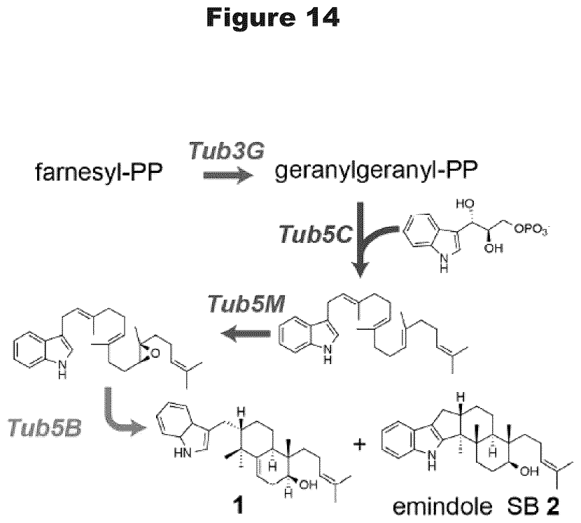

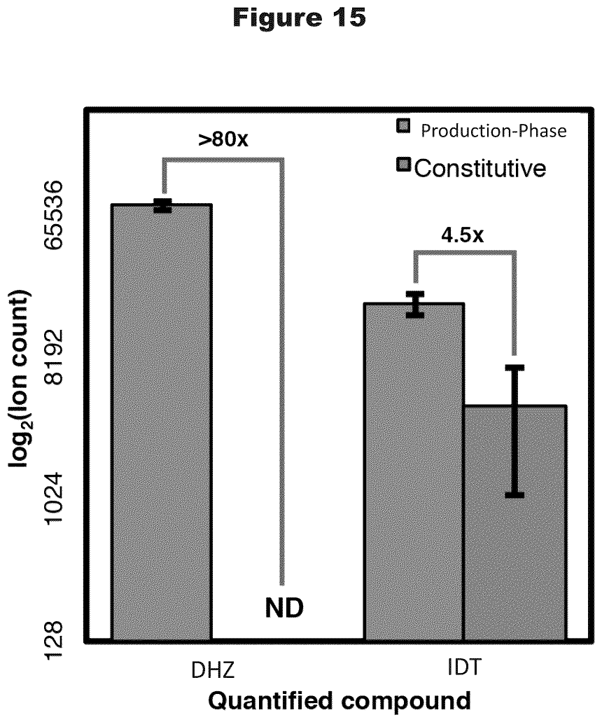

To study the utility of the new promoter set for heterologous expression of a biosynthetic system, production of fungal derived deydrozearalenol (1) and indole-diterpene (2) was examined (FIG. 13, Compounds 1 & 2). The biosynthesis of the indole-diterpene compound the coordinated expression of four in Aspergillus tubingensis genes (FIG. 14, Seq ID Nos. 59-62). Two versions of each pathway were constructed: one having all production-phase promoters, and the other having all constitutive promoters (FIG. 14). The production-phase promoter system utilized the pADH2 from S. cerevisiae (Seq. ID No. 1), pADH2 from S. bayanus (Seq. ID No. 38), and pPCK1 (Seq. ID No. 2) and pMLS1 (Seq ID No. 3) from S. cerevisiae. In the constitutive system, transcription was driven by four frequently used strong constitutive promoters: pTEF1, pFBA1, pPCK1, and pTP11 (Seq. ID Nos. 51-54). Each indole-diterpene system was constructed on a single plasmid harboring four expression cassettes: promoter::GGPPS::tADH2; promoter::PT::tPG11; promoter::FMO::tENO2; and promoter::Cyc::tTEF1; wherein, the promoter sequences corresponded to either the production-phase or the constitutive promoters (FIG. 13). Similar constructs were built for the dehydrozearalenol compound with the two genes HR-PKS and NR-PKS (Seq. ID Nos. 63 and 64). All plasmids were constructed using yeast homologous recombination. It should be noted that pADH2 sequences from S. cerevisiae and S. bayanus (61% identity) are sufficiently unique for this type of assembly. The production of compounds 1 and 2 produced by S. cerevisiae BJ5464/npgA/pRS424 transformed with each of these plasmids were measured over seventy-two hours in YPD batch culture (FIG. 15). An 80-fold and 4.5-fold increase in titer of compound 1 and 2 was observed for the system using the production-phase promoters as compared to the constitutive system.

Materials and Methods Supporting the Production-Phase Promotor Experiments

General techniques, reagents, and strain information: Restriction enzymes were purchased from New England Biolabs (NEB, Ipswich, 25 MA). Cloning was performed in E. coli DH5a. PCR steps were performed using Q5® high-fidelity polymerase (NEB). Yeast dropout media was purchased from MP Biomedicals (Santa Ana, CA) and prepared according to manufacturer specifications. Promoter characterization experiments were performed in BY4741 (MATα, his3Δ1leu2Δ0 met15Δ0 ura3Δ0) while all experiments involving the production of 1 were performed in BJ5464-npgA which is BJ5464 (MATαura3-52 his3Δ200 leu2Δ1 trp1 pep4::HIS3 prb1Δ1.6R can1 GAL) with two copies of pADH2-npgA integrated at δ elements. All Gibson assemblies were performed as previously described using 30 bp assembly overhangs.

Construction and characterization of promoter-eGFP reporter strains: All promoters were defined as the shorter of 500 base pairs upstream of a gene's start codon or the entire 5′ intergenic region. All promoters from S. cerevisiae were amplified from genomic DNA, while ADH2 promoters from all Saccharomyces sensu strictowere ordered as gBlocks from Integrated DNA Technologies (IDT, Coralville, Iowa). Minimal alterations were made to promoters from S. kudriavzevii and S. mikitae in order to meet synthesis specifications. In all constructs, eGFP was cloned directly upstream of the terminator from the CYC1 gene (tCYC1). pRS415 was digested with Sac and Sall and a Notl-eGFP-tCYC1 cassette was inserted by Gibson assembly generating pCH600. Digestion of pCH600 with Accl and Pmll removed the CEN/ARS origin, which was replaced by 500 bp sequences flanking the ho locus using Gibson assembly to yield plasmid pCH600-HOint. Each of the promoters to be analyzed was amplified with appropriate assembly overhangs using primers 9-92 Table S2 and inserted into pCH600-HOint digested with Notl to generate the pCH601 μlasmid series. Digestion of the pCH601 μlasmid series with AscI generated linear integration cassettes which were transformed into S. cerevisiae BY4741 by the LiAc/PEG method. Correct integration was confirmed by PCR amplification of promoters and Sanger sequencing.

For characterization, all strains were initially grown to saturation overnight in 100 μl of YPD media. These cells were then reinoculated at an OD600 of 0.1 into 1 ml of fresh YPD and allowed to grow to OD600=0.4 to reach mid-log phase growth (approximately 6 hrs). 500 μl of each culture was pelleted by centrifugation and resuspended in YPE broth for YPE data while the remaining 500 μl was used for YPD data. The 0 hour time point was collected immediately after resuspension. For each time point, 10 μl of culture was diluted in 2 ml of DI water and sonicated for three short pulses at 35% output on a Branson Sonifier. Expression data were collected for 10000 cells using a FACSCalibur flow cytometer (BD Bioscience) with the FL1 detector. Data were analyzed in R using the flowCore package.

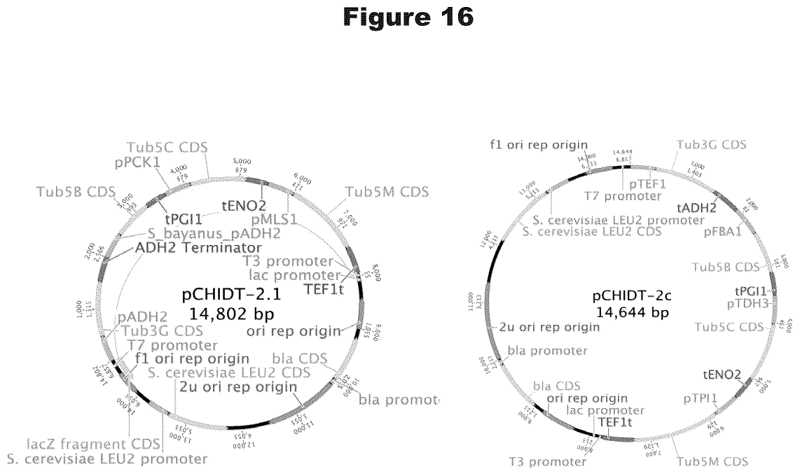

Construction of plasmids to produce compounds in S. cerevisiae: The sequences for genes assembled on IDT producing plasmids are contained in the supporting information. Regulatory cassettes of promoters and terminators were fused using overlap extension PCR. All genes and regulatory cassettes were amplified by PCR, ensuring 60 bases of homology between all adjacent fragments. 500 ng of each purified fragment was combined with 100 ng of pRS425 linearized with Not1 and transformed into S. cerevisiae BJ5464/npgA. Sixteen clones were picked from each assembly plate and grown to saturation in 5 ml CSM-Leu medium. Plasmids were isolated, transformed into E. coli and purified prior to sequence confirmation using the Illumina MiSeq platform. Detailed plasmid maps for pCHIDT-2.1and pCHIDT-2c are shown in FIG. 16 illustrates the primers used and the assembly strategy (Seq. ID Nos. 65 and 66).

Examining the productivity of indole diterpene generating systems Plasmids pCHIDT-2.1 and pCHIDT-2c were transformed into BJ5464/npgA with pRS424 as a source of tryptophan overproduction. Triplicates of each strain were inoculated into CSM—Leu/-Trp medium and grown overnight (OD₆₀₀=2.5-3.0). Each culture was used to inoculate 20 ml cultures in YPD medium at an OD₆₀₀=0.2 and incubated with shaking at 30° C. for 3 days. Every 24 hrs, 2 mis were sampled from each culture. Supernatants were clarified by centrifugation and extracted with 2 ml ethyl acetate (EtOAc). Cell pellets were extracted with 2 ml 50% EtOAc in acetone. 500 μl each of pellet and supernatant extracts were combined and dried in vacuo. Samples were resuspended in 100 μl HPLC grade methanol and LC-MS analysis was conducted on a Shimadzu LC-MS-2020 liquid chromatography mass spectrometer with a Phenomenex Kinetex C18 reverse-phase column (1.7 μm, 100 Å, 100 mm×2.1 mm) with a linear gradient of 15% to 95% acetonitrile (v/v) in water (0.1% formic acid) over 10 min followed by 95% acetonitrile for 7 min at a flow rate of 0.3 mL/min.

TABLE 3
Summary of Sequence Listing

| Sequence | | |
|---|---|---|
| ID No. | Description | Sequence |
| 1 | S. cerevisiae pADH2 | TATCTAAAAATTGCCTTATGATCCGTCTCTCCGGTTACAGCCTGTGTAACTGATTAATCC |
| | | TGCCTTTCTAATCACCATTCTAATGTTTTAATTAAGGGATTTTGTCTTCATTAACGGCTTT |
| | | CGCTCATAAAAATGTTATGACGTTTTGCCCGCAGGCGGGAAACCATCCACTTCACGAG |
| | | ACTGATCTCCTCTGCCGGAACACCGGGCATCTCCAACTTATAAGTTGGAGAAATAAGA |
| | | GAATTTCAGATTGAGAGAATGAAAAAAAAAAAAAAAAAAAAGGCAGAGGAGAGCATAGA |
| | | AATGGGGTTCACTTTTTGGTAAAGCTATAGCATGCCTATCACATATAAATAGAGTGCCA |
| | | GTAGCGACTTTTTTCACACTCGAAATACTCTTACTACTGCTCTCTTGTTGTTTTTATCACT |
| | | TCTTGTTTCTTCTTGGTAAATAGAATATCAAGCTACAAAAAGCATACAATCAACTATCAA |
| | | CTATTAACTATATCGTAATACACA |
| 2 | S. cerevisiae pPCK1 | ATAGGAAAAAACCGAGCTTCCTTTCATCCGGCGCGGCTGTGTTCTACATATCACTGAAG |
| | | CTCCGGGTATTTTAAGTTATACAAGGGAAAGATGCCGGCTAGACTAGCAAGTTTTAGGC |
| | | TGCTTAACATTATGGATAGGCGGATAAAGGGCCCAAACAGGATTGTAAAGCTTAGACG |
| | | CTTCTGGTTGGACAATGGTACGTTTGTGTATTAAGTAAGGCTTGGCTGGGGATAGCAAC |
| | | ATTGGGCAGAGTATAGAAGACCACAAAAAAAAGGTATATAAGGGCAGAGAAGTCTTTGT |
| | | AATGTGTGTAACTTCTCTTCCATGTGTAATCAGTATTTCTACTTACTTCTTAAATATACAG |
| | | AAGTAAGACAGATAACCAACAGCCTTTCCCAGATATACATATATATCTTTATTTCAGCTT |
| | | AAACAATAATTATATTTGTTTAACTCAAAAATAAAAAAAAAAAACCAAACTCACGCAACTA |
| | | ATTATTCCATAATAAAATAACAAC |
| 3 | S. cerevisiae pMLS1 | CCATTGGGCCGATGAAGTTAGTCGACGGATAGAAGCGGTTGTCCCCTTTCCCGGCGA |
| | | GCCGGCAGTCGGGCCGAGGTTCGGATAAATTTTGTATTGTGTTTTGATTCTGTCATGAG |
| | | TATTACTTATGTTCTCTTTAGGTAACCCCAGGTTAATCAATCACAGTTTCATACCGGCTA |
| | | GTATTCAAATTATGACTTTTCTTCTGCAGTGTCAGCCTTACGACGATTATCTATGAGCTT |
| | | TGAATATAGTTTGCCGTGATTCGTATCTTTAATTGGATAATAAAATGCGAAGGATCGATG |
| | | ACCCTTATTATTATTTTTCTACACTGGCTACCGATTTAACTCATCTTCTTGAAAGTATATA |
| | | AGTAACAGTAAAATATACCGTACTTCTGCTAATGTTATTTGTCCCTTATTTTTCTTTTCTT |
| | | GTCTTATGCTATAGTACCTAAGAATAACGACTATTGTTTTGAACTAAACAAAGTAGTAAA |
| | | AGCACATAAAAGAATTAAGAAA |
| 4 | S. cerevisiae pICL1 | ATTTATTGAAAAGTAAATATCTCGTAACCCGGATGCTTTGGGCGGTCGGGTTTTGCTAC |
| | | TCGTCATCCGATGAGAAAAACTGTTCCCTTTTGCCCCAGGTTTCCATTCATCCGAGCGA |
| | | TCACTTATCTGACTTCGTCACTTTTTCATTTCATCCGAAACAATCAAAACTGAAGCCAAT |
| | | CACCACAAAATTAACACTCAACGTCATCTTTCACTACCCTTTACAGAAGAAAATATCCAT |
| | | AGTCCGGACTAGCATCCCAGTATGTGACTCAATATTGGTGCAAAAGAGAAAAGCATAAG |
| | | TCAGTCCAAAGTCCGCCCTTAACCAGGCACATCGGAATTCACAAAACGTTTCTTTATTA |
| | | TATAAAGGAGCTGCTTCACTGGCAAAATTCTTATTATTTGTCTTGGCTTGCTAATTTCAT |
| | | CTTATCCTTTTTTTCTTTTCACACCCAAATACCTAACAATTGAGAGAAAACTCTTAGCATA |
| | | ACATAACAAAAAGTCAACGAAAA |
| 5 | S. cerevisiae | CAAAAAAACAATGGAAGAACAAAGAAAATTTAGCGGAAGTAAAAATAACAGCCGAAAGC |
| | pYLR307C-A | CAAATTCAGGCTTATCTTGCCTACTCTTTCTTTTATCGAATTCCTTTAGGCCGTTGCAAT |
| | | AGAAAAGTAATAAAAACGCATATACGTAAGTTGTAGTCAGTGTAATTGCAATCTATTATG |
| | | CGCATCAGGTGCGCATACTACATCCATTGGTGCACAAAAAAAGGAACGCAGACAAGAA |
| | | AATTATTCAGTTTGCTGTTCGTGATGAGCCATCCCTGAATATGACTAATGTTAATGTTCA |
| | | ATTTGGGATCTTATTTTTTTTTGTGCAGTAATAAGAATCTTTGAAAAAAAACTATATAAGC |
| | | CTATATAGTTTGTAAGATATAAGACAAAACACACCTGCTTTTCCACTACACATTTTCGTT |
| | | ATTATATAAAAAAGACAGCCAAGTATACTTGTCAACAAAATAAACTCATAGCAATTACAC |
| | | TATAAAAACAATAGCATCAAAA |
| 6 | S. cerevisiae | TGGCAATCCCCTCCGATCGTCCGCGGCAAAATGGTCGTCAATCGGACAAAGGGGGAT |
| | pYGR067C | GATGGGATCTGGTAATAGAAGAAAATATGGACTAAAGGTAGCCGCTAAAGCGATCCAG |
| | | GCATGTGTTGCCAATGATGTAAGTCAAGCGAAGGAAATGGTTCAGTAATATGATAGACA |
| | | GACTGCACTTCAAGGGTGCGCCCCCTCCCCCGCGCATATGCTTACAACGCAAAAT/stAT |
| | | TGACGTTTAATGTGGATACTTATCGTAATCGCTGCATTATAGATTTCGAGTCATGTTCAC |
| | | TTAACCCCACATATTTATATAGAACGCATCTTCAAAGTACTTATAAAGTTTAGTTTTACAT |
| | | TTTTCTGCTTTCTATTTCTTCTTTTTCGGTTCTTCTTCATGCCAGTTGGCATGGCTTAAGA |
| | | GCTTTACTTGTCGCTTTTATTTAAAACCTTCTCTCGGGAGAAGACAATTGTTGATATACA |
| | | GTAATTGTATTTGCATTATCACTGCT |
| 7 | S. cerevisiae pIDP2 | AACGTCTATCTATTTATTTTTATAACTCCGGGATGTCATTGCCGGTGGTCCGAAAATCG |
| | | GCAAATAAGGAAATAAGGGAAGAATATGCAGTAGTCAAATCATCAGTGTTCTCTTTGAT |
| | | ACCTTTCAGGGCTAGGAATAGTGGGGGTGGAGTATAATATCAAAAACCGGACTTAACAT |
| | | TATTGGTTCGGTTGGAATTGGCTATAGGCAAACTAGTCTCCGGCATGATATATAAATGA |
| | | CAGCCTGCAATTGTATGTTACTACACTCTTGACTTGTCGACTACAGTCGCTGCTCAGGC |
| | | ACGAGAATAGGAGGTAAGAAGGTAACGTACGTATATATATAAAATCGTA |
| 8 | S. cerevisiae pADY2 | GAGCTCCGTGGAATAGGCGAGCGGCTGAGTGGTTCTCCAAGCTACGGTTTTTACGTGT |
| | | AGCCCCATGTGAGCAAGCCAAACAAGGGCCCTTAAAGGCGTGACTACAAAAAGGGGC |
| | | GGGTTGGAAGGTCATCTGCAGCGAGATACGAAAAGATTTTTTGCCAGATTTGCGGTTG |
| | | GGCGGCTATTTCGGTATTGTTGGGGTAACAAACGTTGGGGAAGACTGCATTTTCTTACA |
| | | GCTTTTTTTCGTTATCGCGGGTTGGGCGGCTATGGCGCCTTCTCCTCTGTACTCCAACC |
| | | TGTCAGAGACACCAAGCTGTATATAAAGCACCTTGGTTGGATCGTATTTCCCTGAGATC |
| | | TTGCTATAGGTTCATTTTATATATCGTCCAATAGCAATAACAATACAACAGAAACTACTA |
| | | GCATCTGTTTATAAGAAAAAGGCAAATAGTCGACAGCTAACACAGATATAACTAAACAA |
| | | CCACAAAACAACTCATATACAAACAAATAAT |
| 9 | S. cerevisiae pGAC1 | CCCTATCTTTTTTTTTTTCTCGCAATCTGGGGAAAGCTTTTCTCATGCTTATACGTGATTT |
| | | GTTATATAAGGGATTGCTATTTCAGGCATCATTCACCTCCTTTTGTATCCTTAGTTTCAC |
| | | TGCATTTGATATATATATATACGTATCTGTAGTTTCCTTCCATTACATAACGCATAATATA |
| | | CTATTTCCATAGTCTATCTTACATCTTTTTTCTTACTTTTGTTAAGGAACGGATAACGATA |
| | | AAACAAAAAGAGAGATTTAAGATTACTTCTGTAACTTTTTTGATCCATTACCAAAACTATA |
| | | TTTTTTTTCTTTTCTCTCCTCTGGCATTAAACACAGTTATTGCTACAGCTAATCATCGATA |
| | | TAATAATACATCACATTAACTGTCTATAAGAGGCTGGTACTTAGTAGATGGTGAGAATTT |
| | | TTTATTTTTGTATTTTAACTTCATTTTTGTAAACAAGTTTGGAACTGGAACTTACTATAGAA |
| | | CAAGAGCTTAAACC |
| 10 | S. cerevisiae pECM13 | GTTGTATCCTATTGGATCACGGGCGACGGACAAGACCCGAAGTGCGGACCGGCATGG |
| | | TCAGCTTGCACGGAAGCTTTAAGGGTTTCCCTTGTTTCGGCATTAGAAGAGGCATTTCG |
| | | CACGTTTTACCGGGTCAGAAACTTCGAGGAAGCTGTGACAATTGGAAAAAAAGGCAAA |
| | | ACTAAATGCAATGTATCCGGTTGCCCATGCATTATTTGTGATGTTTTCGGATGTAGTTCG |
| | | CTGCGCTCCGCGGCGATATATCCTCTAGCGAGAGGCATATGTATAAATATATATATATA |
| | | TATCTAACAAAAGCATTCAAGTTTCTTTCTCTGGTGTTACGTCTTTGTTCGACTTTCTCT |
| | | GCTTACAGCCCTGTATGACCAAAGAAAAAATAAAAAGACAGCTACATACCAGCAGAAAT |
| | | TTTTTATAGTATTACACTATACATCCAAGTTTTTTCACAATTATTTATTGTTTTTCTCACAT |
| | | AGAAAATTCCGCATACTGCGATTATA |
| 11 | S. cerevisiae pFAT3 | GAAAGCTTATTACTGAGTTTTGCGGAGCATCGCTCGGAGCGGCGGAATTGAATCGAAC |
| | | CGCCGTGCTATTACCGAACAAAAAAATTCGAAAGCATAAACTCAGTAGTGAAAAACTTG |
| | | AGAATTTTCAGATGAGTGGCGACTTTCCAGTCCTTGCGGTTTTGTCACCTTAGTCAGCT |
| | | AGTAAGGAGGCCGTGTGGGTTAGAGTGGCTACAATCCTCAAAGGGCACTTCTAGAACC |
| | | CACGGTGAATTTTTTTTGGCATGATAAATCGGTAGAATCGGTGAAGTAATTACCCAAAA |
| | | AAGGATCGGGATTGTGTTTCTCGTAATTCCGTATTATTGCCGATGGCATCGACTACTTC |
| | | TTTTTTCAGAAACCCCAACAAGGGTCTATTGTAATTGTATATAAACCTTTTTGTAATGGAT |
| | | ATATACATGTGGTACTATTTCTCCTCATCCTGCTCCATCGAAAATCCTCATACGAAGAGT |
| | | TAGGAAAGCAAAGAAAACAACAAAAAC |
| 12 | S. cerevisiae pPUT1 | AGACACAATGCGAAAAATCGCGCAGGGACATAATTTTTGTTTTCATTATTCTTTCGCTTA |
| | | TTCCCTCCGTTAGCTCCACCGCTTTTTTGATTGGAATTTCCTTTCGGCAATGGCTTTCC |
| | | GGTTACCACGCCTCGGGTTTCGCATCCCGAAAAGCATATCTACACAAGAAAAATGAATG |
| | | ATAAACAATTGATGAGTGGCGCTATTTCCCTTATCATCTCATTATTGTACTTAGTATCGT |
| | | CTATTATCAGGAGAAATCGCATGAACTAAGCCCATTTTCTCACCCTTCTGCCTTCTTATA |
| | | TAAAGCTTGCTGGGAACCGAACACAAACTCCACAAGTCCGTAGCAGCTCTTCTCTTTTG |
| | | TCTTTTATATATCATAAACATCGCTACATAGTAATAACACTAACGCACGCTAGAA |
| 13 | S. cerevisiae pNQM1 | AGGGGTAGCGGCTTTTTCATCAACTCGATTATTACCCTTTAGAGACCTTCCCTAAAGTG |
| | | AGCGGCAATTATTTCCGGATGTTAGTAGGGTAATATGGTTACGGATTTGTGACACAAAA |
| | | GGGCTTTTCAACAGTCGGTCTGGGTTGAAGGATTTTCAGGATGACGAAGCTTTCAATAA |
| | | GAGGGACTGGACTGTTAACGCGGGGAATTATAGGTTACTTTCCTTGATCTGGCTCTGG |
| | | CTCTGGCTCTGATTTTGGCTCTTGTACTCCTCGGACTTCTTGACTTGTAACGAAATACG |
| | | TCTTTTGTCCTTCTCTTCTTCTTCCATAGTAGGGGCGAATGAGGGGAGCATAGTGGATC |
| | | CTTCTAACCATCTAGAATGGGGTGGACAACATATAAAAGAAGAGCAATCTTGCAGCGCA |
| | | GTCATATTTATGCTAAGTATATCATTATTTCTTGCTAGCGTAAGTCATAAAAAATAGGAAA |
| | | TAATCACATATATACAAGAAATTAAAT |
| 14 | S. cerevisiae pSFC1 | AGCCTAGTCCCGGTAAACCGCAAACGGACCTTAATTGTGACGAAGGGCCCAAATTTGA |
| | | TGGGTCGGTGTTAATGATTAGTCCTCATTGTCATAATAAAGTGTGATGATGGAGGCAAT |
| | | GATGATATACGGTAGTACTACTGCTCGAGGTGCTATCTTTTAACCAATCCTTTGAGATTC |
| | | TTGTCGCCACGGAGTTACTACCTTTTACAAACCGTAATGTCACATTTTGCATATATCTTA |
| | | TGTATAAATATATAGTTCACTTACTACTTGTTCTCGTTTTGTTAACTTTCTTGTTGTAGTT |
| | | CTTCTTGTTCTTGGCGTTTCCCCCTTTGTTTTCTATCTGCTTCATAAGTAAAGTGCAAAG |
| | | CATTTTGGAAGATATTATCAATTGAGTCATTGAAAGAAACTTGGCATCTTCCCTATTACT |
| | | AAAACTAAGAATACTTGATTCAAGAAAGAAGTTTATATTAGTTTTAGCCGTAAGATAACA |
| | | TAACAAAGAAGAAGAAAGAAAA |
| 15 | S. cerevisiae pJEN1 | TCGATCAGCTCCAATTAAATGAAGACTATTCGCCGTACCGTTCCCAGATGGGTGCGAAA |
| | | GTCAGTGATCGAGGAAGTTATTGAGCGCGCGGCTTGAAACTATTTCTCCATCTCAGAG |
| | | CCGCCAAGCCTACCATTATTCTCCACCAGGAAGTTAGTTTGTAAGCTTCTGCACACCAT |
| | | CCGGACGTCCATAATTCTTCACTTAACGGTCTTTTGCCCCCCCTTCTACTATAATGCATT |
| | | AGAACGTTACCTGGTCATTTGGATGGAGATCTAAGTAACACTTACTATCTCCTATGGTA |
| | | CTATCCTTTACCAAAAAAAAAAAAAAAAAAAAAAAAAAAAAAATCAGCAAAGTGAAGTAC |
| | | CCTCTTGATGTATAAATACATTGCACATCATTGTTGAGAAATAGTTTTGGAAGTTGTCTA |
| | | GTTCCTTCTCCCTTAGATCTAAAAGGAAGAAGAGTAACAGTTTCAAAAGTTTTTCCTCAAA |
| | | GAGATTAAATACTGCTACTGAAAAT |
| 16 | S. cerevisiae pSIP18 | ACATAGTACTGTACGATTACTGTACGATTAATCTATCCACTTCAGATGTTCAACAATTCC |
| | | TTTTGGCATTACGTATTAATACTTCATAGGATCGGCACCCTCCCTTAAGCCTCCCCTAAA |
| | | TGCTTTTCGGTACCCCTTTAAGACAACTATCTCTTAACCTTCTGTATTTACTTGCATGTTA |
| | | CGTTGAGTCTCATTGGAGGTTTGCATCATATGTTTAGGTTTTTTTGGAAACGTGGACGG |
| | | CTCATAGTGATTGGTAAATGGGAGTTACGAATAAACGTATCTTAAAGGGAGCGGTATGT |
| | | AAAATGGATAGATGATCATGAATACAGTACGAGGTGTAAAGAATGATGGGACTGAGAG |
| | | GGCAATTATCATCCCTCAGAATCAACATCACAAACATATATAAAGCTCCCAATTCTGCCC |
| | | CAAAGTTTTGTCCCTAGGCATTTTTAATCTTTGTATCTGTGCTCTTTACTTTAGTAGAAAG |
| | | GTATATAAAAAAGTATAGTCAAG |
| 17 | S. cerevisiae pAT02 | AAGTTCTTGACTACCCCTATCTCACACTAGTACGTAATTCAATGTATCATTCGTATTGTA |
| | | AGTAGATAGAGACGCAATACAGGAAAGCTGACCTTCCTTCCAATCACCACGGCTGAAA |
| | | TGCTTTGTTGACCAATTACGGACGCTTAAGAGCGGACGCGGCTGGAACGGCTCCATCC |
| | | TAAATCGGCGGAGGGAGAACTCCGATACCAGCCGACATGGCAATAATAGTGACAGTAG |
| | | ATGCTACCAGCCCCGCAATAATTTCACAGTAGATCATCAACAGTCTCCTCATTTCTGGA |
| | | AATGATCAGCAACTTCGACGGATTTAACTCTCAAGCAGTTACGCACTCCGAGAACAGCC |
| | | GTGATCATCTTTGAACAAGCAAAATATATAAAGCAGGAGAACTGTCCTACCTAGAGCTA |
| | | GAATAGCCATAACTAACTATGTAACATTCTACAGATCAATCAAAAACAATCTTCAATCAC |
| | | AGAAAAAAATAAAAGGC |
| 18 | S. cerevisiae pYIG1 | TTTTCTAGTTCTTCTTCTGCAATATTGCCTTTTGGGAAGAAGGATCGAAAGTAGCCATTT |
| | | GCAGACACGTTTTTACTATATTTACTGTATCTTCGATTGCGCGGCTAAAGTTGCCATATT |
| | | ATTATTATATTGCAGCTCAACCCCGCATTTCCGGAGTTTTCTTTTTTTTTATTTGGGGTAA |
| | | TTTGGAGGTCGGCGGCTATTGGTGGGGCCGGAAATGGTGACACACTTGTAATATATAAG |
| | | GAGGAAATCCTACATGTGTATAAGCGAAATCACAAGGATAATAATGTATTGCTAAACAC |
| | | CCTCAAGAAAGAAAATAATCATAACGAAATC |
| 19 | S. cerevisiae pFBP1 | CGGATGGAATCGCCGCTTTTGAATTCACCTCCGGGGTATTATTATTATTCTTAGTAGTC |
| | | GCGGTCGTGCGGACACCCGGAGTTATGCGGGCCCGAAAGCTCATTATGTAGTAAAGC |
| | | TAGGTAATGTTAAGGGCGTAAGAGCCAACGCAAGGCAGCAATAGCCTGGTATTCCCAC |
| | | ATATCAAGAAAGCTTAAAAAGTTGAGACAGGGAATTTGAAGGCGAAGATTGCCGAACT |
| | | GGCCAATACCCACTACTTTTTTTTTGGTTTGCTTGGTTTCTTCCTGTCGCTTGCCAACTT |
| | | GTGGCATCTTCCCCACACTATATTATAAGGATCGTCCTATGTATAGGCAATATTATCCAT |
| | | TTCACTCGCTAACAAATGTACGTATATATATGGAGCAACAAGTAGTGCAATTACAGACG |
| | | TGTATTTTGTCTTGATCTTGCTTTTTGTATGATAGGCCTAAGAATAACAGTGCGAACATA |
| | | TAAGAAACATCCCTCATACTACCACACAT |
| 20 | S. cerevisiae PHO89 | AGACCTTTTTTTTCTTTTTCTGCTTTTTCGTCATCCCCACGTTGTGCCATTAATTTGTTAG |
| | | TGGGCCCTTAAATGTCGAAATATTGCTAAAAATTGGCCCGAGTCATTGAAAGGCTTTAA |
| | | GAATATACCGTACAAAGGAGTTTATGTAATCTTAATAAATTGCATATGACAATGCAGCAC |
| | | GTGGGAGACAAATAGTAATAATACTAATCTATCAATACTAGATGTCACAGCCACTTTGG |
| | | ATCCTTCTATTATGTAAATCATTAGATTAACTCAGTCAATAGCAGATTTTTTTTACAATGT |
| | | CTACTGGGTGGACATCTCCAAACAATTCATGTCACTAAGCCCGGTTTTCGATATGAAGA |
| | | AAATTATATATAAACCTGCTGAAGATGATCTTTACATTGAGGTTATTTTACATGAATTGTC |
| | | ATAGAATGAGTGACATAGATCAAAGGTGAGAATACTGGAGCGTATCTAATCGAATCAAT |
| | | ATAAACAAAGATTAAGCAAAA |
| 21 | S. cerevisiae CAT2 | TCCGAAGAGCGTGCTACCAATTCTTCATCTCGTTAACAAACTGGTTCTCCGTTAAAAATT |
| | | GTGCTATATGTCCTATAAGCCAACTCTATCTATATCTTTTCTTTTAGTCCTACTTTGGATA |
| | | CTGTTACCACCATTTTAGATTGCTTTTTCTTTTGCCGCTAGCCTTACAATATTTGGCAAA |
| | | CTTTTTTTTTTTAGCCGCCGAGACTCTTGATCTATGGCCGGGCGAAAGGGCAAATGACT |
| | | GCTTATCCCCGCCATCACTTCCCCCCGCCCAAGGGTTTAGAATTGGGGATTAAGTAAA |
| | | AACGAATGACTATTCCTCTCAAAGTCATCCTTGTTCGACAAAAAGAATGGAATATAACAT |
| | | ATTGGAACAATTTCATCCTCTTTTCCCCATTTTCGCATATAAGAGCAACTAAACGCCGGT |
| | | GAGTAAAGTGCCCTTCCCTACAGACTCTTTTACTCAGGTATATATATATATATATCCCTT |
| | | AAAAACTAAAAAGAAAGCACTC |
| 22 | S. cerevisiae CTA1 | AGCGGTTGTTCTAACCACTATTTAAAGCCGCAATTAGTAATGCAAAAAGTTGGCCGGAA |
| | | TTAGCCGCGCAAGTTGGTGGGGTCCCTTAATCCGAAAAAGGACGGCTTTAACAAATAT |
| | | AAACTCCGAAAATCCCCACAGTGACAGAATTGGAGAAACAACCAGTTTTGATATCGCCA |
| | | TACATATAAAGAGATGTAGAAAGCATTCTTCACTGTAATGTCCAAATCGTACATTTGAAT |
| | | TTCTTGTAGGTTTATTTAAAAGGTAAGTTAAATAAATATAATAGTACTTACAAATAAATTT |
| | | GGAACCCTAGAAG |
| 23 | S. cerevisiae ICL2 | AATTTTTATTTTCTCCTTCCATATGAGCGACAGCGGTTACTAGCCGCTGTCCTCAGGTTA |
| | | ATGATCCAAGTCCGAGATCCGGGCCGAATATGCTTGCGGGGAAAGAAATAAAAGTGCA |
| | | TTGGAGAAGAAAAGGATATGCTCTTCAATTAGAAGCGCCGAAACACTAACATCATGCTA |
| | | GCGATATCATACGTACACTATATAATGTAAAAAATGGGCTTAAGAATAACTCTCTTATTT |
| | | CTTAACTTTTGTTGCGGTTGAAGAGCTTATAAAAGTACTAGTGGCCTAAAGAAGCTACA |
| | | GCGCCGATAATAATATCGATTTCGACTTTTCTAGTATTTCGCCG |
| 24 | S. cerevisiae ACS1 | TGTGCACATACGTCCAGAATGATATCAAGATAAATGGCACGTGTATGTACGGCTGTGTA |
| | | AATATGATAATCATCTCGGACGAACGGCGTAGCACTCTCCATCCCCTAAAAATGTTCAC |
| | | GTGTGACTGCTCCATTTCGCCGGATGTCGAGATGACCCCCCCCCCTCAAAAGGCACTC |
| | | ACCTGTTGACATGCCGTGGCAAATGATTGGGGTCATCCTTTTTTTCTGTTATCTCTAAGA |
| | | TCCAAAGAAAAGTAAAAAAAAAAGGTTGGGGTACGAATTGCCGCCGAGCCTCCGATGC |
| | | CATTATTCAATGGGTATTGCAGTTGGGGTACAGTTCCTCGGTGGCAAATAGTTCTCCCT |
| | | TCATTTTGTATATAAACTGGGCGGCTATTCTAAGCATATTTCTCCCTTAGGTTATCTGGT |
| | | AGTACGTTATATCTTGTTCTTATATTTTCTATCTATAAGCAAAACCAAACATATCAAAACT |
| | | ACTAGAAAGACATTGCCCACTGTGCT |
| 25 | S. cerevisiae PDH1 | AATATAAATAAAATTCCATACAGCATGTCTAATCATAGCTAATTTATACATATTCATCATG |
| | | AAAACATATAGGGGAAAATATGGTCGGTTAACACACCTATCAAAAAATTATTCAGCAATT |
| | | CCAATCTCGTTAGTAAAATATATTCTTATTTTTTTTTTTTTTCTCTGATTGTATTATTTCTG |
| | | GAGTTTTGACTTATTTTTTTACCACATCGCGCTTTTCGTCCCCAATCTCTCTGATATATG |
| | | ATGCTGTCTATAGGTAGCCACTTCCCCGATGTCGGACCTCGGGCCGTTTACAAACTTTA |
| | | TTGAGATGACCTTATTTCTCCACATTCTAGTCATTCAACTTTTACCCTCATATGTTTACCT |
| | | TCACTAATGTGAAAGCATGACCAAAGAAAGTGTATAAGGTATATAAATCTGCCATAATGT |
| | | ATGTATAACTTATTAGGACTTTCTCAAATAGTATTTTGGTATTTTCTACTGTTCTCTGATG |
| | | ATCGAGAGCAAACAGA |
| 26 | S. cerevisiae REG2 | AAGTACGATATGGTATAACTGTAACATTGAAGGACTGAAGGACTGAAGGACTGAAGGA |
| | | CTATAGTCAAGGGCCAATGGGGAAGGTCCCTTCCAGGCCATTTGCCCGATAGTTTGTC |
| | | CTTCTCTTGCTTTTCCGACGGCCCGATTGCATGTGGCGGGGCAGCACTGGATAAAAAA |
| | | ACGTGGGGGGAGTGATTAAATTTATACGCTTATTGTGTCAACACGGAAACCTTATAGTT |
| | | ATCATTACTAACATCGCAACAAGCTGCTTTTTTACTCGTTTTTAGCCACACCATACCCCC |
| | | TTTAATTAACTAATAATGCATAAAATAGTTATTGCTTCTTGAGTTGCAGCTTCTTCCTGGA |
| | | CGTACTGTTATATATGGCATGTCTTCGCATGTCCGTCAAATTTAGCGTTGTCTCGAAACT |
| | | TAGGCTGTCGTTCTTGCTGTCTGTCTTCTGATAAAATAATATATTGGAATAAGAAAAAAA |
| | | AAATAGGAACAAGAAAGTGTGTGAGA |
| 27 | S. cerevisiae CIT3 | ATATTATTCAGTTGAAAGACAAAAAAACATAAATATTTCTATGAGCAAACAATTTGAACA |
| | | GAAAAATAAAATTGGGGAAGTGACACACCATGGTAGCGGTTCTAAAGCGAAATCGGCA |
| | | AAGCGGCTAAATAGCAGTTTTGATGACTTACTCCACACTGAAAATGGATGACCTTAAAT |
| | | AGGAGATAAAGCTTTTTCATCCCTATGTATTTAAGATGACTGGCTTGTCAAGCATTCTAA |
| | | TCATAAAAAAAAGATCGTATTTGATCAAGAATTTATACATAGACGCCGCTAAATAATTGA |
| | | ATACAAA |
| 28 | S. cerevisiae CFRC1 | CTCGTTTGCCGTTACATTGCATTGATGGTACAATAAAGGGCATGCTTTATATCGAGATG |
| | | TTTCAGTGTATATGAGGGGAAACAGAAAAGAGTCATTCCTGCCATTTTTTGGTCACTGC |
| | | TTTTTCTGCTATGAGTAATGGTGAAGTTCCTTGTGGCTACACGCTTAATGTCATCGGGT |
| | | TACTGCTCCTAATATCCGCATATAAGCTTTATGCAGGGATCAGTTGGGCGGCTATTTAT |
| | | CTACACCCAGTCATCCGGCGTGACTGGATCTCCACTTGCCGCAATAAGTCGGTGGACA |
| | | AATGGAGATTTAAGAGTAAAGATGCATGATGGTATAATTCCTTTAGTCGAAATAGATATA |
| | | TTTCAAGCGCATATATAGGCAGACGCTTGTACTGTAGAAATAGCCGATATTCAATTGCG |
| | | CTCTATGTGTGTTTTTATTCCAGGTTTTCCTTGGATTCTACGTATTGTACGACTTTCTTAT |
| | | CCTCCACAAACGTCATCGTGTCAGTA |
| 29 | S. cerevisiae RGI2 | CCCAACAGATTTCAAGTCTGTCGCCTTAACCACTCGGCCATAGTGCCTAAAACAATGTA |
| | | GGTTATTTAAGCAAGTATTGTAGATACTTTTCGTAATAAACTACAATGCACCCACGACTC |
| | | GCGGTGTAATGATGGCATGAAATCATTGAACGAAGTTTTGCGGCTATACGGCTGAAGG |
| | | ACGAGACTAAAGGGACAGGAATTATTAATGCGGGGTATAATTTGAATAGTATTAACGGG |
| | | CACTGCCGTTTAGCCATCAAATGCTATTGTTGGGGTATTCTCTCTACTTTTTGTTCTTGG |
| | | CTTGAACCTTTTCGGCGGTTGGCAATCGTCCGTATATAAGCATCGGCTGTCCCAATCCT |
| | | CTATTGCCCTTTTCCCTTGCACCTCCTTCTCAATTCTTCGTATCTTTCGCGTAAAGGTAG |
| | | ATCTTGATTCACCTATCTGTCGAAACACGATTAAGTGCAAACGAAACAACGTACAGTAT |
| | | ATAACAAAGTATTTTAAATAATAAGA |
| 30 | S. cerevisiae PUT4 | GCTATGACGTTTGGGTGGCCTAGCCGGTTCGCGTGTGCCTGTCGCTTTTGTCGCTTTT |
| | | CAACTTCTGCCCGATATTTCCTATCAAAGGAAAATGGGACGTTTTCAACCCCTCGCTAT |
| | | CATCGTGCCTGCACTCTGCCTATCGCCAACTACACCGGGGTTTTATCTGCTTCACCCCT |
| | | CCATCCAGTGCTGATAACAAGAAGAACCTTGCAGGGTAGGGCAGGACCTACGGCCAAA |
| | | ATACTAATTATGTCTGTTTATGTACATGCCCAATCTGAATATTCCATGAATGTAGGCAC |
| | | AGCATATCTCCATCCATGTACTGATACAGACGCATAAACATATATGTATATACATACTTA |
| | | TACACTCGAATATTTGTAGACTGATGTACTTCTATATATATATAGGGGGTTTGTGTTCCT |
| | | CTTCCTTTCCTTTTTTTTTCTCTCTTCCCTTCCAGTTTCTTTTATTCTTTGCTGTTTCGAAG |
| | | AATCACACCATCAATGAATAAATC |
| 31 | S. cerevisiae NCA3 | TAGATGCGCCATCTCCGAGAAAAAATCTAGACAATAACAGCGACAATTAACCTAAAGAG |
| | | GATAGAAGATCGAGCAAAAAAATTTTTTTAATATGGGGTCAGTGGCGATATTATACTATA |
| | | GGAGTTAAAGAGTAAGTTGAGTGTAAGGTGGTAGAATTATGATTGAACTCCGAAACTAA |
| | | GCGCCGATTATGGGTGGCAAAGCGGACAGCTTTTGATATATAATCGATCGCTCTCGTA |
| | | GTTGATATCCTCTCTCTTGCTTATCTTTTCCTGTATATAGTATATGTGTACATACAGATAC |
| | | GAATATACCTCAGTTAGTTTGTTTTAACATTAAATATTCAACAGTTGCCAGTAGCAAAAA |
| | | GAATATATCCATTCATTTCGAGCTTTTTCGTCTCATTACTGATATCCAACTAACAGTCTC |
| | | CTCATAGACGGTACCTTACTTTCCTTTAATATTATAATACTAGTATAGTCGCACATACTTA |
| | | ACTCGTCTCTCTCTAACACATA |
| 32 | S. cerevisiae STL1 | CTACGTCGCCTGTTCGAGCGGCTCTGTTCGTTGCATGAAACTAAAATAAGCGGAAAGT |
| | | GTCCAGCCATCCACTACGTCAGAAAGAAATAATGGTTGTACACTGTTTCTCGGCTATAT |
| | | ACCGTTTTTGGTTGGTTAATCCTCGCCAGGTGCAGCTATTGCGCTTGGCTGCTTCGCG |
| | | ATAGTAGTAATCTGAGAAAGTGCAGATCCCGGTAAGGGAAACACTTTTGGTTCACCTTT |
| | | GATAGGGCTTTCATTGGGGCATTCGTAACAAAAAGGAAGTAGATAGAGAAATTGAGAAA |
| | | GCTTAAGTGAGATGTTTTAGCTTCAATTTTGTCCCCTTCAACGCTGCTTGGCCTTAGAG |
| | | GGTCAGAATTGCAGTTCAGGAGTAGTCACACTCATAGTATATAAACAAGCCCTTTATTG |
| | | ATTTTGAATAATTATTTTGTATACGTGTTCTAGCATACAAGTTAGAATAAATAAAAAATAG |
| | | AAAAATAGAACATAGAAAGTTTTAGACC |
| 33 | S. cerevisiae ALP1 | GAGCTATAGTCTTTTGCGCTTTCAATACGTGTAGCGGTGTACCAAAAGTTGCACAAAAA |
| | | TGTAGTTGTCAATGAAAGCGCACTACGTATATAATGACTATTTTTTTTTTCCTGGGTTGC |
| | | ATGGGTAATTTGTTGTTAATATGCGATTTTCTTGGGGAAAAGGGTGTCATAGCGCCAAA |
| | | AACTGCCGTGCGGCACAGTATGTATGTTTTTGAGTCGCGGCGTTTAAGGGCTTGGCAT |
| | | AAAAAGTGGTTCAAGCGAGTGATAAGTTGGGCGAATGTCGTCTTTTTTGTAACCATGTC |
| | | TTTCCTGAAAACAACCTGTAGGCAGCTCCACTCCACATAAGGGCTTTCTCCAATGGCAA |
| | | TGGGAGCTCGGAACACCGGAGTAGAAATTTTTATAATGTGTATTGTATAAAACTTGCTT |
| | | GTTATGCAGTTTTTGTTTTTTTTGTTACTCTTCCGTAGCACAATAGACATATATTAGCGG |
| | | CAAAATTGTAGTGTTGCGATTATTGCC |
| 34 | S. cerevisiae NDE2 | GTGTAGTATTGATCTTGTTGGTATTGCTAGAAATGCTTCAGCAATACTGTATAAAATATG |
| | | GAAACGTTGCCATGGCAAGACAAAAGAAGTGATCTTGAGTGAAATAATAGAGCCCGGA |
| | | TGGCCGGGTAAATTCAACCGCTCGTACCGTTTATAATACGCATAAACGCCGAAAATGTC |
| | | TCTATTTTAGTCATTCCCCAGAGTGCGGTATTGCGTACACCTGTCATGCGTTCCTTAGT |
| | | GCCGATAGATATACTAATATCGATGCGTCACAGTAGCAGATCATCTCTGACACTTGTTT |
| | | CCCCATTTTTTTTTTTCATTTTTTAAAGGGTTTCTCTACAGCCTACAGGCCTCCCCTAATA |
| | | AGTCAGCCCCTCCCTTTGGAGTGCGCTGTTGACCTGCGTATATAAGAGGTATATCAGT |
| | | GCCAGTAGGTAAACCCATCTTGCGGGGATTGTACCAGGAACATAGTAGAAAGACAAAA |
| | | ACAACCACCGTACTTGCCATTCGTATAG |
| 35 | S. cerevisiae QNQ1 | CATCAATTAGGGCAAACTTGAATAGTCAGCTAGGTCATATATTTAAAATCTTAGCCCT |
| | | ATGACTACATTAGGTTTATTGTTAGGTCTTTACGGCTGCATATTTGCTTTCGCCGTTCGG |
| | | CGGGGTCCTGCGACGATTTCTGCGCGGTCTTGTATGGGTGGAGTTGACAGTTAACCCT |
| | | CCGGACCCCCTACCCCGGTGTGCCCCCGGTCCATCTATCCATTTTGCGGTAACCCCTT |
| | | TGCGCGACAGCTGCTTATCAAGGTACCTGGATCGAGCCATAAAAATTGATCTACACAGA |
| | | TGAGATGGGGCATTGGGATATATTATTAGTCGGAGTATCATTATAGTTATTCAGTTTTAT |
| | | GCAGGTTACTGGCCAAACGTTTTTCTTCATTTGGAATAATCGTTTAGGAGCTACTGTTC |
| | | CGGTATAAAGTAACAAGCACAGTAGCAGAGTAATACGCAGTGACGATAATAGAGACTA |
| | | GTAAAACAGTCGAGTTGTCGGACCTAAA |
| 36 | S. paradoxus pADH2 | TAGTCTTATCTAAAAATTGCCTTTATAGTCCGTCTCTCCAGTCACGGCCTGTGTAACTGA |
| | | TTAATCCTGCCTTTCTAATCACCATTCTACTGTTTAATTAAGGGATTTTGTCTTCATCAAC |
| | | GGCTTCCGCCCAAAAAAAAGTATGACGTTTTGCCCGCAGGCGTGAAGCTGCCCATCTT |
| | | CACGGGCCTGACCTCCTCTGCCGGAACACCGGCCATCTCCAACTCATAAATTGGAGAA |
| | | ATAAGAGAATTTCAGATTTTCAGAGGATGAAAAAAAAAAGGTAGAGAGCATAAAAATGG |
| | | GGTTCACTTTTTGGCAAAGTTACAGTATGCTTATTACATATAAATAGAGTGCCGATAATG |
| | | GCTTTTTTTCATCTTCGAAATACGCTTGCTACTGCTCTTCCAGCGTTTTTATTACTTCTTT |
| | | CTTGTTTCTCCTTAGTATATAAAATATCAAGCTACAACAAGCATACAATCAACTGTCAAC |
| | | TGTCAATTATATTATAATACACT |
| 37 | S. kudriavzevii pADH2 | CTCTCAAATCTTTTAGCGCCAAGGACTCCAACTAATTGTATCTTGAATTTGCCTTTACGA |
| | | TCCGTTTGTCCAGTCACGGCATGTATATCTTATTAATCCTGCCTTTCTAATCACGTATTC |
| | | TAATGTTCAATTAAGGGATTTTATCTTCATCAACGGCTCCCACGCAAAAAATGACGTTTT |
| | | GCACACAGACACGAAATACACCTTCCACCGGAACAACGGCCATCTCCAACTTATAAGTT |
| | | GGGGAAATAAGACAATTTCAGACTTCAGAGAATGAAAAAAAAAAAAGGTACATCACAGA |
| | | TGGGGTTCAGGTTTGCTACAATTGCAGGGAGCCTGTCACATATAAATAGACCTCCAGT |
| | | GATGATATCTTTCAGTCTTCAAACGTCTCTTGTCACAGTTCTGGTCGTTCTATATCACAT |
| | | CTCTCTTGGTTCTACTTATTGTCTATAATATCAAGCTACAGCAAGCATACAATCAACTAT |
| | | CTACCATACCATAATACACA |
| 38 | S. bayanus pADH2 | GATCCAGTTCTCCAGTGACACAGCCTTTATCTGGTCAAACCTTTCTTTCTAATCACCTAT |
| | | GCTGATGCTTAATTAAGGGATTTTTGTCTCCATCAACGGCATGCGCCCAAAAATGACGT |
| | | TTTTTTTAACCCATAGACACGAAACTACCCATTTTCCACCGGCCTGACCTACCACCGGA |
| | | ACAACGGCCATCTCCAACTTGCAAGTTGGGGAAATTAAGAGCATCGCAGGTTTAATGG |
| | | AAGAAAAAAAAAAGGTACAGCACAGCGCAAATGGAGTTAGTTCCCTTATGTCACACACT |
| | | CACACACAGTCGGTCAGATCAAGCATACTGGGTGCGTATAAATAGAGTGGCCATTGCC |
| | | ACCCTGTTTATCTCAAAATCTGTCTTGTTAGTCTGTCTTCTCCCTTTTTCAGGTTACAATT |
| | | CTCTTGTTTCTACTTAGTATATAAGTATATCAAGCTATATTAAGCATACTATCAACTGTCA |
| | | ACTCTATCCTCAAAATACAATACAAA |
| 39 | S. mikitae pADH2 | TTTCCCAAAAAGTATTATTTTTAAGTGATAATTGATAAAAGGGGCAAAACGTAGACGCAA |
| | | ATAAAACGGAAATAATGATTCTCAGACCTTTTAGCGTCAAGAACTGCAACTAATCTTATC |
| | | TTAAAATTATCTTTATAATCCGTTTCTCCCGTCACAGTCTGTGTATCTGATTAATCCTGC |
| | | CTTTCTAATCACCTATTCTAATGTTCAATTAAGGGATTTTGTCTTCACCAACGGCTTCCA |
| | | CCCAAAAGTAAAAAATGACGTTGTACCCACAGACATCTTCACCGGCCTGACCTGCCAC |
| | | CGGAACAACGGCCATCTCCAACTCATAAATTGGAGAAATAAGAGAATTTCAGATTCTGG |
| | | AGGATGAAAAAAAAAAAGGTACAGCATAAATGGGGTTTTATGTGGGTACAATTACACTA |
| | | GGACTATCACATATAAATAGACGGGCAATGTAGGTTCTTTTCCACCCTTGAGACAGAGT |
| | | TATTC |
| 40 | S. castellii pADH2 | TGTCGTGGACGAAATACGCCACAATTTTGCCGAGAAGGTCATTAGTATGTCCAAGAAAC |
| | | CCTAGGTGTAAAGTCGGGAAATCCGAATCTCCGATTTTGGAGGGGCCCATGCCCTACT |
| | | TTTTTTCGCCAGGGGTGAAATTCCAAACCCGTGCGCGTTCTTGGAATTTGACAGCGCAT |
| | | TGAGTATGTGCTGCGTATTCCCACTATCATGACGCGCCCTTTATCTGGGAAAAATGGAA |
| | | CTGGATGCTGAAATATTTCACTCTCAGATCACATATCCCAAATCCIGTGAGTGAATTGTT |
| | | TGGTCAGGCGACCAAACAGGAATATGGAATAGATTCTATTCTCTGGATTCTACAATTAT |
| | | CCATTGTTAGCAAAACAAAAAAAACTGGTGGTATATATATTCAGAGCCTAAAATTTAAAG |
| | | GTTGGATCTCAATTTTAAAAGTTTTCATTCTGTTTTGTTTTTGTTTCTTCTTAGCTCACGA |
| | | ATAACCAAACAAAAAACAATCAATA |
| 41 | S. paradoxus pPCK1 | CAATAGGAAAAAACCAAGCTTCCTTTCATCCGGCACGGCTGTGTTGTACATATCACTGA |
| | | AGCTCCGGGTATTTTAAGTTATACAAGAGAAATATGCGGGCTAGACTAGCAAGATTCTG |
| | | GACTGTATAACGTTGTGGATAGGCGGATAAAGGGCCCAAACAGGATTGTAAAGCTTAG |
| | | ACGCCTCTGGTTGGGCAATGGCATGTTTGTGTATTAAGTAAGACTTGGCTGCGGGATA |
| | | GCAAAACTGAGCAGAATATAGAAGGCCACAAAAAAAAGGTATATAAGGGCAGCAAAGT |
| | | CTTTATAATATATGTAGATTCTCTTCTCTGTGTAATTCATTCTTGTGCTTACCACTCAAAT |
| | | ATACAGAAGTAAGACAGATAACCAACAGCCTTTCCCAGATATACATATATCTCATTGTTT |
| | | CAGTTTAAACAATAATCATATTTGTTCTCAAAAATAAAAAAAAACTAAACTCACTCAA |
| | | TCAATCATTCCATAAAAAAAAACAAT |
| 42 | S. kudriavzevii pPCK1 | CTTCCTTTCATCCGGCACGGCTGTGTCCCCACATCTCCCTAAAGCTCCGGGTATTTTAA |
| | | GTTATACAAGGGAAATATACGGGCTGGACTACAACTTGCAGGTTGCACAGCGTTATGG |
| | | ATAGGCGGATAAAGGGCCCAAGCAAGATCGTGAAGCTTGGACGCGTCTGGTTGGACA |
| | | ATGGTGACTTTTTGTGTATTAGATAATGCTTGACTGGAGAATATCAGGACTGAGCAGAG |
| | | TTAGGAAGACCACAAAAAAGGTATATAAGGGCAACGTCTCCGTGATATGGATAGG |
| | | CTCTTCTCTCTGGTTACAATTCATTATTTCAGTTGTTFGCTAGATATAGAGATATAATACA |
| | | TCTAATAAACAGTCACTTCCAGAGATATATATATATACATATATCTATCTCCTCCTCCCA |
| | | GCTTAAATAATAACTATATTTGTTTAACTCGAAGAAAAAAAAAATTCAAATTTACTCTATC |
| | | AATTCAATTACCTCATAAAAAACAATA |
| 43 | S. bayanus pPCK1 | CTTCCTTTCATCCGGCACGGCTGTGTCCCCACATCTCCCTAAAGCTCCGGGTATTTTAA |
| | | GTTATACAAGGGAAATATACGGGCTGGACTACAACTTGCAGGTTGCACAGCGTTATGG |
| | | ATAGGCGGATAAAGGGCCCAAGCAAGATCGTGAAGCTTGGACGCGTCTGGTTGGACA |
| | | ATGGTGACTTTTTGTGTATTAGATAATGCTTGACTGGAGAATATCAGGACTGAGCAGAG |
| | | TTAGGAAGACCACAAAAAAGGTATATAAGGGCAACAAAGTCTCCGTGATATGGATAGG |
| | | CTCTTCTCTCTGGTTACAATTCATTATTTCAGTTGTTTGCTAGATATAGAGATATAATACA |
| | | TCTAATAAACAGTCACTTCCAGAGATATATATATATACATATATCTATCTCCTCCTCCCA |
| | | GCTTAAATAATAACTATATTTGTTTAACTCGAAGAAAAAAAAAATTCAAATTTACTCTATC |
| | | AATTCAATTACCTCATAAAAAACAATA |
| 44 | S. paradoxus pMLS1 | CGATACCACACGGTCCATTGGGCCGGTGGTGTTAGTCGACGGATATATGCATCTGTCC |
| | | CCTTTCCCGGCGAGCCGGCAGTCGGGCCGAGGTTCGGATAAATTTTTGCATTGTATTA |
| | | GTTTCTGTCATGAGTATTACTTATGGTTCCTTTAGAGCTAATCATTAGCTCGGTACCGGC |
| | | TGTTATGCAATTTATGACTTTTCTTCTACAGTGTCAGCCTTGTGACGATTATCTATGAAC |
| | | TTTGGATGTAGCGCATCGAGATTCGTATCTTTCATTGGATAGTAAATGGGAAGGATCGA |
| | | TGACCCTTATTACATTCTTTCCTATACTTAATATCCATTTAATCTATCTTCTTGAAAGTATA |
| | | TAAGTAACGGTAAATTTACCATACTTATGCTATTCTCATTTATCCCCTAATTTTCTTTTAA |
| | | CTTCTCGCCCTACAGTAACTAAGAATAACGGCTACTGTTTCGAAATTAAGCAAAGTAGT |
| | | AAAGCACATAAAAGAATAAAGAA |
| 45 | S. kudriavzevii praS1 | AGACCGAAGCGGGTAATGGACGGAATTAAGCAATTGTCCCCTCTCCCGGGGAGCCGA |
| | | CAGTCGGACCGAGCTTCGGATAAATTTCTGTATTGTTTFTGTTTCCGTCATGGGTATTAT |
| | | TTTCGGGATCCTTTTGCCAACCCCATAGTCAATCGTTAACATTTACCGGCTATGTA |
| | | GGATTATGACTATTCTCCTGCATGATCAGCGGAAGTGACGATTATCTATTAATTTTGAAC |
| | | TTCTACTTCGTGATCCGGAATTTAATTGGATAATAATGTGTCCGAAGGATCGAGTGACC |
| | | CTTATATTCTGTAGTTTTTTGTTACTGGCCATCCAATTCGTGTTCTTGGAAGTATATAAGT |
| | | TACAGTCGATTGACCTTTCTCAAGCTATTTTCATCTTTCTCCTACATTTACGTTTCTCTTC |
| | | TTCAATACAGCAGCTAGAAGTTACGATTACTCCTGTGATAAACAAAGTAATAGTAG |
| | | CCCACAAAAAGAGAGAAAGTAAAA |
| 46 | S. bayanus pMLS1 | GTAGCAGTCCGGAAATAAGCAAATGTCCCCTTTCCCGAGCTAACCAACGGTCGGGCCG |
| | | AGCCTCGGATAAATTTTTGCTTTGTTTTTGTTTCTGTCATGGGTATTATACATCATTTATT |
| | | TAGTTAACCCCTAGACTAATTAGCCGGCCATTAGTATGTAAGATTATGACTATAGTTTGT |
| | | ACCGGAACCCTGGTAGCAACTACTCATGAACTTTGGGCTCAGTATTTCGCAATCCCGG |
| | | TTTTAATTGGATAGCCTATCGCGAAGGATCGATGGATGACCCTTAGAATTGTCTCTTTT |
| | | GTTACTACTCATTCAATGCGTGTGCTCTTGCAAGTTATATAAGTCACTCTAAATTAGTTTA |
| | | TACTTGAGCTTTTTACATTTCTCCCTTGATTGTTTCTTTCTCTTTTCCCCTTGTTCTGGTT |
| | | TATTGTAATAGCTAAGTGCAACGATTACCGCTGTTAAGTTAAAGAAGAGAGACAAGTAA |
| | | TAATAGTACACAGCAAGGAAAAAA |
| 47 | S. paradoxus pICL1 | TTACTAAATAGGCTGGCATCAGCTAACCCGGATGGTTGAATCCGGCTTTTGCTACTTGT |
| | | TGTCCGATGAAAAGGAGCGGCTTCCCTTTTGCCCCAGATTTCCATTCATCCGAGAGGT |
| | | CGCTTATCAGACTTCGTCATTTCTCATTTCATCCGAGATGATCAAAATTGAAGCCAATCA |
| | | CCACAAAACTAACACTTAACGTCATGTTACACTACCOTTTACAGAAGAAAATATCCATAG |
| | | TCCGGACTAACATTCCAGTATGTGACTCAATATTGGTGCAAATGAGAAAATCATAGCAG |
| | | TCAGCCCAAGTCCGCCCTTTACCAGGGCACCGTAATTCACGAAACGTTTCTTTATTATA |
| | | TAAAGGAGCTACTTTACTAGCAAAATTCTTGTAATTCCTCTTCCCTTGCTAACTTCTTCTT |
| | | GTTTTCTTTTCCTTTTTACACACAGATATATAACAATTGAGAGAAAAACTCTAGTATAACA |
| | | TAACAAAAAAGTCAACGAAAAAA |
| 48 | S. kudriavzevii pICL1 | GTTACGGTGCCGCGCCGGTGGCCGGTGGTCTTCCGGTAAACAAAAAAAGCTGCCTCC |
| | | CTTTCGCCCCAGATTTCCATTCATCCGAGGGCACCGCTTGTCAGACTTTATCGTTTTCC |
| | | TCATTTCATCCGAGAAGATCAATTCAAAGGCAATGACCACAAAAGCAACTCCTAACGTT |
| | | GTGTTACGCTACCCTTTACACAAAATATTCATAACCCGTAATGAATCCTAAGGTATGTGA |
| | | CTCAATTTTGGTGTAGAAAATGAGGAAAACGTAATACTAAGTTAAAGCTCGCCCTTTAAA |
| | | GTGAATATTCCTTGACCATTTGCGCAGGCACACCCGAATTCACAAACGTTTCTTTATTAT |
| | | ATAAAGGACCAGCTCTGCTAGTCAAATTTTTATAACTGCTTGTTCAGTTGCTGCTTCTTT |
| | | CTTGTCAATTTATTTCTTGTACTGTTCAACTACATAAAGCAAAGAGAAAACTCTCAGAAT |
| | | AACATAACAAAGAAGTCAACGAAAA |
| 49 | S. bayanus pICL1 | ACGAGGCTCGGCGTTTACTGCTGAATTTCCGGAAAGAAAGGGAAGGTTCCCTTTACCC |
| | | CAGATTTCCATTCATCCGAAGGACTGCTTATCAGAATTTGACATTTTTCTCATTTTATCC |
| | | GAGAAGATCAATTTAAGGCTAGTGACCACAAAACTAACTCTCATGCTGCGCTACCGCAA |
| | | GTTTCGCTCACAGAAAGAAAGCAAGCACCCATAGTCCGGACTACATCCTTGTATGTGAC |
| | | TCAAATTTTTGGCGTTGCCAATTAAACTGAAGTGTAAAGATTACTTCAAGCTCACCCTTT |
| | | AAAGTAGAATTCCTTAACGGTTTTAAATAGACACACCGAAATTAATAAACACTTTCTTTAT |
| | | TATATAAAGGACAGAGTTTATTACTGGAATTCTCTTAACGCCTTCCTCCCTTACTATTGT |
| | | ATCTTTTCCTTTCACATAATCGCTACATAACTACATAGAGAAAACTCTCAGATTAACACA |
| | | GTAACAACGAAGAAAACAAAAAA |
| 50 | S. cerevisiae pTDH3 | ACAGTTTATTCCTGGCATCCACTAAATATAATGGAGCCCGCTTTTTAAGCTGGCATCCA |
| | | GAAAAAAAAAGAATCCCAGCACCAAAATATTGTTTTCTTCACCAACCATCAGTTCATAGG |
| | | TCCATTCTCTTAGCGCAACTACAGAGAACAGGGGCACAAACAGGCAAAAAACGGGCAC |
| | | AACCTCAATGGAGTGATGCAACCTGCCTGGAGTAAATGATGACACAAGGCAATTGACC |
| | | CACGCATGTATCTATCTCATTTTCTTACACCTTCTATTACCTTCTGCTCTCTCTGATTTGG |
| | | AAAAAGCTGAAAAAAAAGGTTGAAACCAGTTCCCTGAAATTATTCCCCTACTTGACTAAT |
| | | AAGTATATAAAGACGGTAGGTATTGATTGTAATTCTGTAAATCTATTTCTTAAACTTCTTA |
| | | AATTCTACTTTTATAGTTAGTCTTTTTTTTAGTTTTAAAACACCAAGAACTTAGTTTCGAAT |
| | | AAACACACATAAACAAACAAA |
| 51 | S. cerevisiae pTEF1 | ATAGCTTCAAAATGTTTCTACTCCTTTTTTACTCTTCCAGATTTTCTCGGACTCCGCGCA |
| | | TCGCCGTACCACTTCAAAACACCCAAGCACAGCATACTAAATTTCCCCTCTTTCTTCCT |
| | | CTAGGGTGTCGTTAATTACCCGTACTAAAGGTTTGGAAAAGAAAAAAGAGACCGCCTC |
| | | GTTTCTTTTTCTTCGTCGAAAAAGGCAATAAAAATTTTTATCACGTTTCTTTTTCTTGAAA |
| | | ATTTTTTTTTTTGATTTTTTTCTCTTTCGATGACCTCCCATTGATATTTAAGTTAATAAACG |
| | | GTCTTCAATTTCTCAAGTTTCAGTTTCATTTTTCTTGTTCTATTACAACTTTTTTTACTTCT |
| | | TGCTCATTAGAAAGAAAGCATAGCAATCTAATCTAAGTTTTAATTACAAA |
| 52 | S. cerevisiae pFBA1 | TGGGTCATTACGTAAATAATGATAGGAATGGGATTCTTCTATTTTTCCTTTTTCCATTCTA |
| | | GCAGCCGTCGGGAAAACGTGGCATCCTCTCTTTCGGGCTCAATTGGAGTCACGCTGCC |
| | | GTGAGCATCCTCTCTTTCCATATCTAACAACTGAGCACGTAACCAATGGAAAAGCATGA |
| | | GCTTAGCGTTGCTCCAAAAAAGTATTGGATGGTTAATACCATTTGTCTGTTCTCTTCTGA |
| | | CTTTGACTCCTCAAAAAAAAAAAATCTACAATCAACAGATCGCTTCAATTACGCCCTCAC |
| | | AAAAACTTTTTTCCTTCTTCTTCGCCCACGTTAAATTTTATCCCTCATGTTGTCTAACGGA |
| | | TTTCTGCACTTGATTTATTATAAAAAGACAAAGACATAATACTTCTCTATCAATTTCAGTT |
| | | ATTGTTCTTCCTTGCGTTATTCTTCTGTTCTTCTTTTTCTTTTGTCATATATAACCATAACC |
| | | AAGTAATACATATTCAAA |
| 53 | S. cerevisiae pPDC1 | CATGCGACTGGGTGAGCATATGTTCCGCTGATGTGATGTGCAAGATAAACAAGCAAGG |
| | | CAGAAACTAACTTCTTCTTCATGTAATAAACACACCCCGCGTTTATTTACCTATCTCTAA |
| | | ACTTCAACACCTTATATCATAACTAATATTTCTTGAGATAAGCACACTGCACCCATACCT |
| | | TCCTTAAAAACGTAGCTTCCAGTTTTTGGTGGTTCCGGCTTCCTTCCCGATTCCGCCCG |
| | | CTAAACGCATATTTTTGTTGCCTGGTGGCATTTGCAAAATGCATAACCTATGCATTTAAA |
| | | AGATTATGTATGCTCTTCTGACTTTTCGTGTGATGAGGCTCGTGGAAAAAATGAATAATT |
| | | TATGAATTTGAGAACAATTTTGTGTTGTTACGGTATTTTACTATGGAATAATCAATCAATT |
| | | GAGGATTTTATGCAAATATCGTTTGAATATTTTTCCGACCCTTTGAGTACTTTTCTTCATA |
| | | ATTGCATAATATTGTCCGCTGCCCCTTTTTCTGTTAGACGGTGTCTTGATCTACTTGCTA |
| | | TCGTTCAACACCACCTTATTTTCTAACTATTTTTTTTTTAGCTCATTTGAATCAGCTTATG |
| | | GTGATGGCACATTTTTGCATAAACCTAGCTGTCCTCGTTGAACATAGGAAAAAAAAATAT |
| | | ATAAACAAGGCTCTTTCACTCTCCTTGCAATCAGATTTGGGTTTGTTCCCTTTATTTTCA |
| | | TATTTCTTGTCATATTCCTTTCTCAATTATTATTTTCTACTCATAACCTCACGCAAAATAA |
| | | CACAGTCAAATCAATCAAA |
| 54 | S. cerevisiae pTPI1 | TATATCTAGGAACCCATCAGGTTGGTGGAAGATTACCCGTTCTAAGACTTTTCAGCTTC |
| | | CTCTATTGATGTTACACCTGGACACCCCTTTTCTGGCATCCAGTTTTTAATCTTCAGTGG |
| | | CATGTGAGATTCTCCGAAATTAATTAAAGCAATCACACAATTCTCTCGGATACCACCTC |
| | | GGTTGAAACTGACAGGTGGTTTGTTACGCATGCTAATGCAAAGGAGCCTATATACCTTT |
| | | GGCTCGGCTGCTGTAACAGGGAATATAAAGGGCAGCATAATTTAGGAGTTTAGTGAAC |
| | | TTGCAACATTTACTATTTTCCCTTCTTACGTAAATATTTTTCTTTTTAATTCTAAATCAATC |
| | | TTTTTCAATTTTTTGTTTGTATTCTTTTCTTGCTTAAATCTATAACTACAAAAAACACATAC |
| | | ATAAACTAAAA |
| 55 | S. cerevisiae tADH2 | GCGGATCTOTTATGTCTTTACGATTTATAGTTTTCATTATCAAGTATGCCTATATTAGTAT |
| | | ATAGCATCTTTAGATGACAGTGTTCGAAGTTTCACGAATAAAAGATAATATTCTACTTTTT |
| | | GCTCCCACCGCGTTTGCTAGCACGAGTGAACACCATCCCTCGCCTGTGAGTTGTACCC |
| | | ATTCCTCTAAACTGTAGACATGGTAGCTTCAGCAGTGTTCGTTATGTACGGCATCCTCC |
| | | AACAAACAGTCGOTTATAGTTTGTCCTGCTCCTCTGAATCGTCTCCCTCGATATTTCTCA |
| | | TTTTCCTTCGCATGCCAGCATTGAAATGATCGAAGTTCAATGATGAAACGGTAATTCTTC |
| | | TGTCATTTACTCATCTCATCTCATCAAGTTATATAATTCTATACGGATGTAATTTTTCACT |
| | | TTTCGTCTTGACGTCCACCCTATAATTTCAATTATTGAACCCTCAC |
| 56 | S. cerevisiae tPGI1 | ACAAATCGCTCTTAAATATATACCTAAAGAACATTAAAGCTATATTATAAGCAAAGATAC |
| | | GTAAATTTTGCTTATATTATTATACACATATCATATTTCTATATTTTTAAGATTTGGTTATA |
| | | TAATGTACGTAATGCAAAGGAAATAAATTTTATACATTATTGAACAGCGTCCAAGTAACT |
| | | ACATTATGTGCACTAATAGTTTAGCGTCGTGAAGACTTTATTGTGTCGCGAAAAGTAAAA |
| | | ATTTTAAAAATTAGAGCACCTTGAACTTGCGAAAAAGGTTCTCATCAACTGTTTAAAAGG |
| | | AGGATATCAGGTCCTATTTCTGACAAACAATATACAAATTTAGTTTCAAAGATGAATCAG |
| | | TGCGCGAAGGACATAACTCA |
| 57 | S. cerevisiae tENO2 | AGTGCTTTTAACTAAGAATTATTAGTCTTTTCTGCTTATTTTTTCATCATAGTTTAGCA |
| | | CTTTATATTAACGAATAGTTTATGAATCTATTTAGGTTTAAAAATTGATACAGTTTTATAA |
| | | GTTACTTTTTCAAAGACTCGTGCTGTCTATTGCATAATGCACTGGAAGGGGAAAAAAAA |
| | | GGTGCACACGCGTGGCTTTTTCTTGAATTTGCAGTTTGAAAAATAACTACATGGATGAT |
| | | AAGAAAACATGGAGTACAGTCACTTTGAGAACCTTCAATCAGCTGGTAACGTCTTCGTT |
| | | AATTGGATACTCAAAAAAGATGGATAGCATGAATCACAAGATGGAAGGAAATGCGGGC |
| | | CACGACCACAGTGATATGCATATGGGAGATGGAGATGATACCT |
| 58 | S. cerevisiae tTEF1 | GGAGATTGATAAGACTTTTCTAGTTGCATATCTTTTATATTTAAATCTTATCTATTAGTTA |
| | | ATTTTTTGTAATTTATCCTTATATATAGTCTGGTTATTCTAAAATATCATTTCAGTATCTAA |
| | | AAATTCCCCTCTTTTTTCAGTTATATCTTAACAGGCGACAGTCCAAATGTTGATTTATCC |
| | | CAGTCCGATTCATCAGGGTTGTGAAGCATTTTGTCAATGGTCGAAATCACATCAGTAAT |
| | | AGTGCCTCTTACTTGCCTCATAGAATTTCTTTCTCTTAACGTCACCGTTTGGTCTTTTAT |
| | | AGTTTCGAAATCTATGGTGATACCAAATGGTGTTCCCAATTCATCGTTACGGGCGTATT |
| | | TTTTACCAATTGAAGTATTGGAATCGTCAATTTTAAAGTATATCTCTCTTTTACGTAAAGC |
| | | CTGCGAGATCCTCTTAAGTATAGCGGGGAAGCCATCGTTATTCGATATTGTCGTAACAA |
| | | ATACTTTGATCGGCGCTAT |
| 59 | A. tubingensis GGPPS | ATGCTGGGATTCCCAATGTTCAACCCAGCTACGCCTGATGTCTGGAAGATGAATACCC |
| | | CTTACTTTCCATTTGTTACACCGGGGTTATTTCCTGCCTCAGCACCCCCATCGCCCACC |
| | | AACGTAGATGCCGAAGCTGCCAGTTCCCAACAGTCGGAAGCAAGCTATCTGGATAAGG |
| | | AGAAAATTGTTCGAGGGCCACTTGATTATCTTCTCAAATCCCCTGGAAAAGACATTCGT |
| | | CGGAAATTCATTCACGCGTTCAATGAATGGCTGCGCATTCCTGAGGACAAGTTGAATAT |
| | | TATCACGGAAATTGTTGGATTGCTTCACACGGCCTCCCTTCTAATCGACGATATTCAGG |
| | | ACAATTCCAAGCTTCGACGCGGCCTCCCAGTGGCCCATAGCATATTTGGTATTGCGCA |
| | | GACAATTAACTCTGCCAATTATGCGTACTTTCTAGCCCAGGAAAGGCTCCGCGAACTGA |
| | | ATCATCCTGAAGCGTACGAAATATACACAGAGGAACTGCTTCGTCTGCACCGCGGTCA |
| | | AGGTATGGACTTGTACTGGCGGGACTGCCTAACCTGTCCCACAGAGGAGGACTATATT |
| | | GAGATGATCGCCAACAAGACTGGTGGCCTATTTCGACTGGCGATTAAGCTTATGCAGT |
| | | TGGAAAGCACTTTGTGCAGCAATGTCATTGAACTAGCAGACTTGTTGGGCGTGATCTTT |
| | | CAGATTCGGGATGATTACCAAAACTTACAGAGTGGACTATACGCCAAGAACAAGGGATT |
| | | TTGCGAGGATTTGACGGAGGGAAAATTTTCCTTTCTGATTATCCACAGTATTAACAGTAA |
| | | CCCGAACAATCACCATCTGCTAAATATACTACGGCAGCGGAGCGAGGACGATTCGGTG |
| | | AAGAAGTATGCTGTTGATTATATCGACTCGACGGGGAGTTTTGACTACTGCCGGGAAC |
| | | GGCTCGCTTCCTTATTGGAAGAGGCGGATCAAATGGTTAAGAAGTTGGAAAATGAGGG |
| | | GGGACAATCAAAGGGGATCTACGATATTCTGAGCTTTCTGTCGTGA |
| 60 | A. tubingensis PT | ATGGATGGGTTCGACCATTCTACIGCTCCACCAGGATATAACGAGCTAAAATGGCTCG |
| | | CCGATATCTTCGTCATCGGAATGGCTGTTGGCTGGGTTGCTCACTATATGGAGATGATT |
| | | CACACGTCGTTCAAGGACCAAACATACTGCATGACCATCGGGGGCCTTTGCATCAATTT |
| | | TGCCTGGGAAATCATATTCTGCACAATGTATCCTGCCAAAGGATTTGTCGAGCGGGTTG |
| | | CCTTTCTCATGGGCATTTCTCTCGACCTTGGGGTTATTTACGCGGGAATCAAGAACGCC |
| | | CCAAATGAATGGCACCACTCTGCAATGGTGAGGGACCATATGCCCCTTGTCTTCGCAG |
| | | CAACGACACTTTGTTGTCTGAGCGGTCATATGGCTCTTACTGCCCAGGTTGGTCCCGC |
| | | ACAAGCCTATACGTGGGGGGCAATTGCATGCCAGCTCTTTATCAGCATAGGGAATGTG |
| | | TTTCAATTGTTGAGTCGGGGAAACACACGAGGGGCGTCATGGACGCTATGGACCTCCA |
| | | GGTTTTTTGGATCAACATCAGCCATTGGCTTTGCTCTTGTTCGATATATTCGCTGGTGG |
| | | GAGGCCTTTTCTTGGTTGAACTGCCCGCTTGTGATATGGTCCGTGGCCATGTTCTTTCT |
| | | GTTTGAAACACTCTATGGAGCCCTATTCTATTCTGTCAAGCGACAAGAAGGGAGATCCC |
| | | AGCGTGGAATCAAGCACAAAGAGAGGTAG |
| 61 | A. tubingensis FMO | ATGGCGGCACTTCCGGACGTTGCCTCCATTCCCATCCCTCTGGTGGCAACCCTAGGCA |
| | | TTGCCCCTCTAATTTTCTATCTCGTCCTTGATAGAATTAGCCCCTTGTGGCCAAATTCCA |
| | | AAGCTTTCCTGATTGGCAAGAAGACCGGAGACCGTGACATCGTTCGAGTGCCCATA |
| | | TGCCTACATCCGTCAGATCTATGGGAAGTATCACTGGGAGCCATTCGTACAGAAGCTG |
| | | TCTCCGAGGCTTAAGGATGAGGATCCGGCCAAATATAAGATGGTTCTGGAGATAATGG |
| | | ATGCAATCCACCTGTGTCTGATGCTAGTTGACGATATAACTGACAATAGCGACTATCGA |
| | | AAAGGCAAGCCAGCAGCCCACCGGATATATGGCCCTTCAGAGACAGCAAATCGCGCTT |
| | | ACTACCGAGTCACCCAGATTCTAAACAAGACCGTGCAAAAGTTCCCCAAGCTGGCCAA |
| | | GTTCCTGCTTCAGAATCTGGAAGAAATTCTCGAAGGCCAAGACCTGTCACTAATCTGGC |
| | | GACGGGATGGACTGGGTAGCCTTTCGACTGTTCCTGATGAGCGAGTTGCAGCCTATCG |
| | | CAAGATGGCGTCATTGAAAACTGGGGCGTTATTCCGGCTGCTGGGGCAATTGGTGATG |
| | | GAGGACCAATCGATGGACGGGACGATGACTACTCTTGCGTGGTGCTCTCAGCTGCAG |
| | | AATGACTGCAAGAATGTCTACTCATCTGAATATGCTAAGGCCAAAGGGGCGCTTGCCG |
| | | AAGACCTCCGAAATCGAGAGCTCTCATTTCCAATTATCCTCGCGCTGGAAGCTCCTGAA |
| | | GGGCATTGGGTCGCCAGTGCTTTGGAGACCAGCTCACCGCGCAACATTCGCAAGGCG |
| | | CTTGCTGTGATTCAGAGTGAGAGAGTGCGCAATGCTTGTTTCAAGGAGCTCAAGTCGG |
| | | CGAGTGCTTCGGTCCAGGACTGGTTGGCTATTTGGGGACGGAACGAGAAAATGAACTT |
| | | GAAGAGCCAGCAGACGTAG |
| 62 | A. tubingensis Cyc | ATGGCCAATGCCCAGCAACCCCCCTTTCGCATCCTTATTGTGGGCGGTTCTGTCGCAG |
| | | GCCTCATCCTTGCGCACTGTCTCGAACGCGCCAATATAGAGTACCTCATACTCGAAAAA |
| | | GGAGAAGATGTTGCTCCACAAGTTGGTGCCTCGATAGGTATCATGCCAAATGGCGGAC |
| | | GGATCCTCGAGCAACTGGGCCTATTTGGGGAGATTGAGCGTGTGATCGAGCCGTTGC |
| | | ATCAGGCGAATATCAGCTATCCAGATGGGTTCTGCTTTAGTAACGTCTATCCTAAGGTT |
| | | CTTGGCGACAGGTTCGGATACCCGGTTGCATTCTTGGACCGGCAGAAGTTCCTGCAGA |
| | | TTGCATATGAGGGGCTGAGAAAGAAGCAGAATGTTCTCACCGGTAAAAGGGTAGTTGG |
| | | ACTGCGACAGTCGGATCAAGGGACTGCTGTTTCTGTGGCTGACGGGACAGAGTATGA |
| | | GGCGGATCTCGTGGTTGGTGCTGATGGAGTACATAGTCGGGTGAGAAGTGAGATTTG |
| | | GAAGATGGCGGAAGAGAATCAGCCTGCATCAGTTTCGACACGTGAAAGAAGAAGCATG |
| | | ACTGTTGAATATGTCTGCGTTTTCGGGATTTCATCAGCCATCCCAGGGCTCGAGATAAG |
| | | CGAACAGATCAACGGTATTTTCGACCATCTATCCATTCTAACAATCCATGGCAGACATG |
| | | GTCGCGTGTTCTGGTTCGTGATCCAGAAGCTGGATAGGAAGTACGTCTATCCTGATGT |
| | | CCCGCGATTCTCAGACGAGGATGCCGTACAGCTCTTCGATCGGGTCAAACACGTGCG |
| | | GTTCTGGAAAAACATCTGTGTGGGGGACTTGTGGAAGAACAGAGAGGTGTCCTCGATG |
| | | ACAGCGCTGGAGGAGGGAGTGTTCGAGACATGGCATCATGATAGGATGGTTTTGATTG |
| | | GAGATAGCGTTCACAAGATGACGCCCAACTTTGGCCAAGGAGCTAATTCAGCCATCGA |
| | | GGATGCTGCCGCGCTCTCTTCCCTTCTACATGATCTCGTCAACGCCCGTGGAGTTTGC |
| | | AAGCCATCGAATGTCCAGATTCAGCATCTCCTCAAGCAGTATCGGGAGACCCGATACA |
| | | CTCGCATGGTAGGCATGTGTCGCACCGCGGCTTCAGTCTCTCGGATTCAGGCCCGAG |
| | | ATGGCATCCTCAACACCGTCTTTGGACGATATTGGGCACCTTATGCTGGCAACCTGCC |
| | | TGCTGACCTGGCATCAAAAGTGATGGCAGATGCAGAGGTTGTTACTTTTCTGCCCTTGC |
| | | CAGGGCGCTCAGGACCGGGCTGGGAGATGTACAGACGAAAGGGGAAGGGAGGGCAG |
| | | GTGCAATGGGTGCTTATAATCTTAAGCTTACTTACGATTGGTGGATTGTGCATCTGGCT |
| | | ACAAAGCAATGCGTTGAGTAGATAA |
| 63 | H. subiculosis hpm8 | ATGCCTTCTACCAGCAATCCATCTCACGTCCCTGTGGCCATCATCGGCCTGGCATGCC |
| | | GATTCCCAGGCGAGGCCACCTCACCATCAAAATTCTGGGATCTTCTTAAGAATGGACG |
| | | AGATGCCTACTCACCAAATACCGATCGATATAACGCTGATGCCTTTTACCATCCCAAGG |
| | | CAAGCAACCGCCAAAACGTGCTGGCAACTAAGGGCGGCCACTTCCTCAAACAGGACC |
| | | CATACGTTTTTGACGCCGCTTTCTTTAACATCACAGCCGCTGAGGCCATCTCCTTTGAC |
| | | CCCAAGCAGCGAATTGCCATGGAAGTTGTCTACGAGGCTCTAGAAAATGCCGGAAAGA |
| | | CACTACCCAAGGTGGCGGGCACACAAACTGCTTGCTATATCGGCTCTTCCATGAGTGA |
| | | TTACCGAGACGCTGTTGTGCGTGACTTTGGAAACAGCCCCAAGTATCATATCCTGGGA |
| | | ACATGCGAGGAGATGATTTCAAATCGTGTGTCCCATTTCTTGGATATTCACGGCCCCAG |
| | | TGCCACCATTCATACAGCCTGCTCATCAAGTCTTGTTGCTACACACTTGGCTTGCCAAA |
| | | GTTTGCAATCTGGAGAGTCAGAAATGGCCATCGCTGGTGGTGTTGGTATGATCATCAC |
| | | CCCTGATGGTAATATGCATCTTACAACTTGGGATTCTTGAACCCCGAGGGCCACTCCC |
| | | GGTCATTTGATGAGAATGCTGGTGGTTACGGTCGTGGTGAGGGTTGCGGTATCCTCAT |
| | | CCTCAAGCGGCTAGACAGAGCTCTCGAAGATGGTGATTCCATTCGCGCCGTCATTCGA |
| | | GCCTCTGGTGTCAACTCTGATGGCTGGACACAGGGTGTCACCATGCCCTCCAGCCAAG |
| | | CCCAGTCTGCCCTTATCAAATACGTATACGAATCGCATGGCCTGGATTATGGTGCGACT |
| | | CAATACGTTGAGGCTCACGGTACTGGTACCAAAGCCGGTGATCCCGCAGAGATTGGC |
| | | GCCCTCCACCGCACAATTGGACAGGGCGCGTCCAAGTCTCGAAGGCTTTGGATTGGC |
| | | AGTGTCAAGCCAAACATTGGCCATCTTGAAGCCGCCGCCGGTGTGGCTGGTATCATTA |
| | | AGGGCGTCCTGTCCATGGAACACGGCATGATTCCTCCAAACATTTACTTCTCCAAGCC |
| | | CAACCCTGCCATCCCTCTTGACGAGTGGAACATGGCCGTGCCTACCAAGTTGACTCCC |
| | | TGGCCCGCCAGCCAAACTGGTCGCCGTATGAGTGTCAGCGGTTTCGGTATGGGTGGT |
| | | ACCAACGGCCACGTCGTCCTTGAGGCCTACAAGCCCCAAGGAAAGCTCACCAACGGC |
| | | CATACCAACGGCATCACCAATGGAATCCACAAGACTCGCCACAGCGGCAAGAGGCTTT |
| | | TCGTCCTCAGCGCCCAGGATCAAGCTGGCTTCAAGCGTTTGGGTAACGCCCTGGTGG |
| | | AGCATCTCGATGCCCTGGGCCCTGCCGCTGCCACCCCTGAGTTCCTCGCCAACCTCTC |
| | | CCACACTCTTGCCGTTGGCAGATCTGGCTTGGCTTGGAGGTCCAGCATCATCGCTGAG |
| | | AGCGCCCCTGATCTTCGGGAGAAGCTGGCAACTGATCCGGGTGAGGGAGCCGCTCGT |
| | | TCTTCAGGCAGCGAGCCCCGTATTGGATTCGTCTTCACGGGTCAAGGTGCTCAGTGGG |
| | | CCCGCATGGGCGTTGAGTTGTTGGAGCGCCCCGTCTTCAAGGCTTCCGTGATTAAGTC |
| | | CGCGGAGACTTTGAAGGAGCTCGGCTGTGAATGGGACCCTATCGTTGAGCTTTCCAAG |
| | | CCTCAAGCTGAGTCTCGACTTGGTGTTCCTGAAATCTCACAGCCCATCTGCACAGTCCT |
| | | ACAAGTCGCCTTGGTTGATGAGTTGAAGCACTGGGGTGTATCACCTTCCAAGGTGGTC |
| | | GGTCACTCCAGTGGTGAAATCGGTGCCGCATACAGCATTGGCGCTCTTTCTCACCGTG |
| | | ACGCTGTCGCCGCTGCTTACTTCAGGGGCAAGTCTTCCAACGGAGCCAAGAAGCTTGG |
| | | TGGTGGTATGATGGCTGTTGGGTGCTCTCGTGAGGACGCTGACAAGCTCCTCTCTGAG |
| | | ACCAAGCTCAAGGGCGGTGTTGCTACCGTCGCATGTGTCAACTCCCCCTCCAGCGTGA |
| | | CCATCTCAGGCGATGCCACTGCTCTCGAGGAACTCCGAGTTATTCTCGAGGAGAAGAG |
| | | TGTGTTTGCTCGAAGACTCAAGGTCGACGTTGCCTACCACTCTGCCCACATGAACGCT |
| | | GTCTTTGCCGAATACTCTGCTGCGATTGCCCACATTGAGCCCGCTCAGGCAGTTGAAG |
| | | GTGGACCGATTATGGTCTCCAGTGTCACTGGTAGCGAAGTCGACTCTGAGCTTCTCGG |
| | | CCCTTACTACTGGACCCGTAACTTGATCTCTCCCGTCTTATTCGCCGACGCTGTCAAGG |
| | | AATTGGTTACCCCTGCTGATGGCGACGGCCAAAACACCGTCGATCTCCTGATTGAGAT |
| | | TGGTCCTCACAGCGCTCTTGGTGGCCCTGTTGAGCAGATTCTGTCCCATAACGGCATC |
| | | AAGAATGTTGCTTACAGATCTGCTCTTACTCGTGGCGAGAACGCTGTTGACTGCAGCCT |
| | | CAAGCTTGCTGGCGAGCTCTTCCTTCTCGGCGTGCCCTTTGAGTTGCAAAAGGCCAAC |
| | | GGTGACTCTGGTTCTCGCATGCTCACTAACCTACCTCCTTATCCTTGGAACCACTCCAA |
| | | GTCATTCCGTGCCGACTCTCGTCTCCACCGTGAGCATCTGGAGCAGAAATTCCCTACT |
| | | AGGAGTCTCATCGGTGCACCTGTCCCCATGATGGCAGAGAGCGAGTACACATGGCGC |
| | | AACTTCATCCGTCTCGCTGACGAGCCTTGGCTCCGTGGTCACACTGTCGGTACCACCG |
| | | TTCTGTTTCCTGGTGCCGGTATCGTGAGCATCATCTTGGAAGCTGCTCAACAGCTGGT |
| | | GGATACCGGCAAGACCGTTCGGGGCTTCCGAATGCGCGATGTCAACCTCTTCGCCGC |
| | | CATGGCTCTCCCCGAGGACCTGGCTACTGAGGTTATCATCCACATCCGACCTCACCTT |
| | | ATCTCTACTGTTGGATCAACCGCCCCCGGTGGATGGTGGGAGTGGACTGTTTCCTCCT |
| | | GCGTCGGAACTGACCAGCTGCGAGACAATGCTCGCGGTCTGGTAGCCATTGACTACG |
| | | AAGAGAGCCGCAGCGAGCAGATCAACGCCGAGGACAAAGCGTTGGTTGCTTCTCAGG |
| | | TCGCGGACTACCACAAGATCCTCAGCGAATGCCCTGAGCATTATGCTCATGACAAGTT |
| | | CTACCAGCACATGACCAAGGCCTCTTGGAGCTACGGCGAGCTCTTCCAGGGTGTGGA |
| | | GAATGTCCGTCCTGGATACGGAAAGACCATCTTTGACATCAGAGTCATTGACATTGGTG |
| | | AGACCTTTAGCAAGGGACAACTTGAGCGACCTTTCCTCATCAACGCTGCCACTCTCGAT |
| | | GCTGTATTCCAGAGCTGGCTCGGCAGTACCTACAACAACGGTGCTTTCGAGTTTGACA |
| | | AGCCCTTCGTTCCCACCTCTATTGGCGAGTTGGAAATCTCTGTCAACATTCCCGGTGAT |
| | | GGCGACTACCTCATGCCAGGCCACTGCCGCTCTGAGCGATACGGCTTCAACGAGTTGT |
| | | CTGCTGATATTGCCATCTTCGACAAGGATCTGAAGAATGTGTTCCTTTCAGTGAAGGAT |
| | | TTCCGAACTTCCGAGCTTGATATGGATTCCGGCAAGGGAGACGGAGATGCCGCTCACG |
| | | TCGACCCTGCCGATATCAACTCGGAGGTTAAGTGGAACTACGCTCTTGGCCTCCTCAA |
| | | GTCCGAGGAAATCACCGAGCTGGTCACCAAGGTCGCCAGCAATGACAAGCTCGCCGA |
| | | GCTTCTCCGTCTGACACTTCACAACAACCCTGCTGCCACTGTCATCGAGCTTGTTTCTG |
| | | ATGAGAGCAAGATCTCTGGCGCATCTTCTGCCAAGCTGTCCAAGGGCCTTATCCTCCC |
| | | CAGCCAGATCCGTTACGTAGTTGTCAACCCTGAGGCAGCGGACGCCGACTCTTTCTTC |
| | | AAATTCTTCTCCCTTGGTGAGGATGGTGCCCCTGTCGCTGCTGAAAGGGGCCCCGCC |
| | | GAACTGTTGATCGCCTCCAGCGAAGTCACTGACGCGGCTGTCCTTGAGCGCCTGATTA |
| | | CCTTGGCCAAGCCTGATGCCAGCATTCTTGTTGCTGTCAACAACAAGACTACCGCCGC |
| | | TGCCCTCTCAGCCAAGGCGTTCCGTGTTGTCACCAGCATCCAGGACAGCAAGTCCATT |
| | | GCTCTCTACACTAGCAAGAAGGCGCCTGCCGCCGACACCTCCAAGCTCGAGGCCATC |
| | | ATCCTCAAGCCAACCACTGCTCAACCTGCCGCCCAGAATTTCGCCTCCATCCTCCAGA |
| | | AGGCACTCGAGCTCCAGGGCTACTCTGTCGTTTCTCAGCCATGGGGCACCGACATCGA |
| | | CGTCAACGATGCCAAGGGAAAGACCTACATTTCTCTGTTGGAGCTTGAGCAGCCTCTG |
| | | CTCGACAACCTCTCCAAGTCCGACTTCGAGAACCTCCGCGCAGTCGTTTTGAACTGCG |
| | | AGCGTCTCCTGTGGGTCACAGCAGGTGACAACCCATCTTTCGGCATGGTTGATGGTTT |
| | | CGCTCGCTGCATCATGAGCGAAATTGCCAGCACCAAGTTCCAGGTCCTGCATTTGAGC |
| | | GCTGCAACTGGTCTGAAGTACGGATCTTCTCTCGCCACCCGCATTCTCCAGTCGGATA |
| | | GCACCGACAACGAGTACCGGGAGGTCGATGGTGCTCTCCAGGTGGCCCGTATCTTCA |
| | | AGAGCTACAACGAGAACGAGAGTCTCCGCCACCACCTCGAGGATACCACCAGCGTTGT |
| | | GACTCTTGCTGACCAGGAGGATGCTCTGCGCCTCACTATTGGCAAGCCTGGTCTTTTG |
| | | GATACTTTGAAGTTTGTCCCCGATGAGCGTATGCTCCCACCTCTCCAGGATCACGAGG |
| | | TTGAAATCCAGGTCAAGGCTACTGGTCTGAACTTCCGAGACATCATGGCTTGCATGGG |
| | | TCTTATTCCTGTTCGATCTCTGGGCCAGGAGGCCAGTGGCATCGTCCTCAGAACCGGT |
| | | GCGAAGGCTACCAACTTCAAGCCTGGCGACCGTGTTTGCACCATGAACGTCGGAACAC |
| | | ATGCCACCAAGATCCGAGCCGACTACCGTGTCATGACAAAGATCCCCGACTCCATGAC |
| | | CTTTGAAGAAGCTGCCTCGGTTGCTGTTGTTCACACCACCGCCTACTACGCCTTCATCA |
| | | CCATCGCCAAGCTTCGCAAGGGCCAGTCCGTCTTGATCCACGCCGCCGCTGGTGGTG |
| | | TTGGCCAAGCAGCCATTCAGTTGGCCAAGCATCTCGGCCTCATCACCTATGTTACCGT |
| | | AGGTACTGAAGACAAGCGCCAGCTCATTCGGGAGCAGTATGGCATTCCCGACGAGCA |
| | | CATCTTCAACTCCCGTGATGCCAGCTTCGTCAAGGGTGTCCAGCGTGTTACCAACGGT |
| | | CGCGGTGTCGACTGCGTTCTCAACTCTCTATCCGGTGAGCTCCTGCGTGCTTCTTGGG |
| | | GATGCCTTGCTACCTTTGGTCATTTCATCGAAATTGGTCTCCGTGATATCACCAACAAC |
| | | ATGCGTCTTGACATGCGACCTTTCCGCAAGAGCACCTCCTTCACATTCATCAACACCCA |
| | | CACTCTCTTCGAGGAAGACCCCGCTGCGTTGGGAGATATTCTCAACGAGTCCTTCAAG |
| | | CTCATGTTCGCTGGCGCCCTTACCGCTCCTAGCCCCTTGAATGCCTATCCCATTGGCC |
| | | AGGTCGAGGAGGCCTTCCGAACCATGCAGCAGGGCAAGCACCGCGGTAAGATGGTGC |
| | | TGTCCTTCTCCGATGACGCAAAGGCTCCCGTGTTGCGCAAAGCGAAGGATTCCTTGAA |
| | | ACTGGACCCTGACGCCACTTACCTCTTTGTTGGTGGTCTTGGTGGTCTGGGTCGCAGT |
| | | CTTGCCAAGGAGTTTGTTGCGTCTGGCGCCCGCAACATTGCCTTCTTATCCCGATCCG |
| | | GTGACACTACCGCCCAGGCCAAGGCTATCGTGGACGAATTGGCTGGCCAGGGTATCC |
| | | AGGTCAAGGCCTATCGTGGTGATATCGCCAGCGAGGCATCCTTCCTCCAGGCTATGGA |
| | | GCAATGCTCTCAGGATCTCCCGCCCGTAAAGGGTGTGATCCAGATGGCCATGGTTCTC |
| | | CGCGATATCGTCTTTGAGAAGATGTCGTACGATGAGTGGACCGTCCCCGTTGGCCCCA |
| | | AGGTCCAAGGTTCATGGAACTTGCACAAGTACTTCAGTCATGAGCGACCTCTTGACTTC |
| | | ATGGTCATCTGCTCCTCAAGCTCCGGTATCTACGGTTATCCCAGTCAGGCTCAATACGC |
| | | CGCTGGCAACACTTACCAGGATGCCTTGGCTCACTACCGTCGCTCTCAGGGCCTGAAC |
| | | GCCATCTCCGTCAACTTGGGTATCATGCGAGATGTCGGTGTCCTGGCTGAGACGGGTA |
| | | CCACTGGTAACATCAAGCTCTGGGAAGAGGTCTTGGGCATCCGCGAGCCTGCCTTCCA |
| | | CGCTCTCATGAAGAGCTTGATCAACCATCAGCAGCGTGGGTCTGGGGACTACCCGGC |
| | | GCAGGTCTGCACTGGTCTTGGTACTGCTGACATTATGGCTACTCACGGCCTGGCCCGG |
| | | CCCGAGTATTTCAATGACCCCCGTTTTGGACCCCTTGCCGTCACCACTGTCGCGACCG |
| | | ATGCTTCAGCTGACGGCCAGGGCTCTGCTGTCTCGCTCGCCTCTAGGCTCTCCAAGGT |
| | | TTCCACCAAGGATGAAGCTGCCGAGATCATTACCGATGCTCTGGTCAACAAGACGGCA |
| | | GACATCCTGCAGATGCCCCCCTCTGAAGTCGACCCCGGCCGACCTCTGTACCGTTATG |
| | | GTGTTGACTCCCTTGTGGCGCTTGAGGTGCGAAACTGGATCACAAGGGAGATGAAGG |
| | | CGAACATGGCGCTGCTGGAGATTCTGGCAGCCGTCCCCATTGAGAGCTTCGCTGTCAA |
| | | GATTGCTGAGAAGAGCAAGTTGGTTACTGTTTAA |
| 64 | H. subiculosis hpm3 | ATGGTGACTGTACCACAGACTATCCTCTACTTTGGAGATCAGACAGACTCCTGGGTTGA |
| | | TTCCCTCGATCAGCTATACAGACAAGCCGCTACGATACCATGGCTACAGACGTTTCTCG |
| | | ACGACCTTGTAAAGGTCTTCAAGGAAGAGTCCCGGGGCATGGATCATGCGTTACAAGA |
| | | CAGTGTTGGTGAATACTCTACACTACTCGACTTGGCGGATAGATACCGCCATGGCACC |
| | | GACGAGATTGGTATGGTGCGTGCTGTCTTGCTACATGCCGCGAGAGGAGGCATGCTAT |
| | | TACAATGGGTGAAGAAAGAATCACAGCTTGTGGACCTCAATGGCTCCAAGCCTGAAGC |
| | | ACTCGGTATCTCTGGAGGACTCACCAACCTCGCAGCACTGGCGATATCCACAGACTTC |
| | | GAGTCTCTATATGACGCAGTCATTGAGGCTGCGAGAATATTTGTCAGATTATGCCGTTT |
| | | TACTTCGGTACGATCAAGAGCTATGGAGGACCGACCTGGCGTTTGGGGCTGGGCAGT |
| | | GCTGGGAATTACACCAGAGGAACTGAGCAAAGTGCTTGAGCAGTTCCAATCCAGCATG |
| | | GGGATTCCTGCCATCAAGAGAGCTAAGGTTGGCGTAACAGGAGACCGATGGAGCACC |
| | | GTTATTGGGCCACCCTCAGTCTTGGACCTATTCATCCACCAGTGTCCCGCTGTGCGCA |
| | | ACCTCCCCAAGAATGAATTGAGCATCCACGCCCTTCAGCACACAGTCACAGTCACAGA |
| | | GGCTGACCTCGACTTCATTGTCGGGAGTGCTGAGCTTCTTAGTCACCCCATTGTGCCA |
| | | GACTTCAAAGTCTGGGGAATGGATGATCCTGTGGCATCCTACCAGAACTGGGGAGAAA |
| | | TGCTAAGAGCAATCGTCACTCAAGTTTTGTCCAAGCCTTTGGACATTACCAAGGTGATT |
| | | GCGCAACTCAACACTCACCTCGGCCCTCGTCATGTCGACGTCCGAGTCATCGGACCTA |
| | | GCAGCCACACCCCCTACTTGGCGAGTTCGCTCAAAGCTGCTGGCAGCAAGGCTATTTT |
| | | CCAGACCGATAAGACTCTTGAGCAGTTACAGCCGAAGAAACTCCCCCCGGGCCGCATC |
| | | GCCATTGTCGGTATGGCTGGCCGTGGTCCTGGCTGCGAGAATGTTGATGAGTTCTGG |
| | | GACGTCATTATGGCGAAGCAGGATCGTTGTGAAGAGATTCCCAAAGATCGCTTCGACA |
| | | TCAATGAGTTCTACTGTACCGAGCACGGGGAGGGTTGCACCACCACCACAAAATACGG |
| | | CTGCTTCATGAACAAGCCTGGAAACTTTGACTCCCGCTTCTTCCACGTGTCGCCTCGTG |
| | | AGGCGCTGTTGATGGACCCCGGTCACAGGCAGTTCATGATGAGCACTTATGAAGCTCT |
| | | TGAGACGGCAGGATACTCTGATGGCCAGACTAGGGACGTTGATCCTAATAGGATCGCG |
| | | GCGTTCTATGGCCAGTCCAACGATGATTGGCATATGGTGAGCCATTATACCCTGGGTT |
| | | GTGATGCCTACACCCTGCAGGGGGCGCAAAGAGCCTTCGGCGCTGGTCGCATCGCCT |
| | | TCCACTTCAAGTGGGAGGGCCCAACATACTCGCTCGATTCTGCATGTGCCTCCACCTC |
| | | CTCTGCTATTCACCTGGCCTGCGTGAGTCTTCTATCCAAAGATGTGGACATGGCTGTTG |
| | | TGGGTGCTGCCAACGTCGTCGGGTATCCTCACTCCTGGACAAGTCTTAGCAAGTCTGG |
| | | TGTCTTGTCCGACACTGGAAACTGCAAAACCTACTGCGATGATGCTGATGGTTACTGCC |
| | | GAGCAGACTTTGTCGGCTCAGTTGTGCTGAAGCGTCTCGAAGATGCTGTCGAGCAAAA |
| | | CGACAACATCTTGGCTGTCGTGGCTGGTTCAGGCAGAAACCACTCCGGCAACTCTTCA |
| | | TCCATCACCACGTCGGATGCCGGTGCCCAGGAGAGACTGTTTCACAAGATTATGCACA |
| | | GCGCCAGAGTCTCTCCTGATGAGATCTCATATGTTGAGATGCACGGCACTGGAACTCA |
| | | GATTGGCGATCCGGCCGAGATGAGTGCTGTTACCAATGTCTTCAGGAAGAGGAAGGC |
| | | GAATAACCCCCTAACTGTTGGTGGAATCAAAGCGAACGTCGGGCATGCTGAAGCTTCT |
| | | GCTGGCATGGCCTCCCTGCTCAAATGCATACAGATGTTCCAGAAAGATATTATGCCCC |
| | | CTCAGGCTCGAATGCCCCATACTCTCAACCCAAAGTATCCGAGTCTTTCTGAGCTTAAC |
| | | ATTCATATCCCCTCCGAGCCGAAGGAGTTCAAGGCTATCGGCGAGCGGCCACGACGC |
| | | ATCCTCCTTAATAACTTTGACGCAGCAGGTGGCAACGCCTCTCTCATTCTGGAAGACTT |
| | | CCCCTCCACCGTCAAGGAAAATGCGGACCCCAGGCCAAGCCATGTCATCGTTTCCTCT |
| | | GCCAAAACACAATCCTCATATCACGCGAATAAGCGTAACCTCCTGAAGTGGCTACGCA |
| | | AGAACAAAGATGCTAAACTCGAAGATGTTGCATACACAACCACCGCCCGCAGAATGCA |
| | | CCACCCCCTCAGATTCTCTTGCAGTGCCTCCACAACGGAGGAGCTCATTTCCAAGCTT |
| | | GAGGCAGACACGGCAGATGCAACTGCGTCTCGGGGCTCGCCCGTTGTCTTCGTATTC |
| | | ACGGGACAGGGCTCTCACTACGCCGGCATGGGTGCCGAGTTGTACAAGACATGCCCT |
| | | GCTTTCCGCGAGGAAGTCAACCTCTGTGCCAGCATCTCTGAGGAGCACGGGTTCCCC |
| | | CCGTACGTGGATATCATCACCAACAAAGATGTTGACATAACCACCAAGGACACCATGCA |
| | | GACACAGCTCGCTGTTGTCACGCTGGAGATCGCCCTCGCCGCATTCTGGAAGGCGTC |
| | | TGGTATCCAGCCGTCAGCAGTCATGGGTCACTCCCTGGGCGAGTATGTGGCTCTCCAG |
| | | GTCGCAGGGGTCCTATCTCTAGCTGATCTGCTCTACCTCGTCGGCAATCGGGCCCGTC |
| | | TCCTGCTGGAGCGCTGCGAAGCCGACACCTGCGCTATGTTGGCAGTATCAAGCTCTGC |
| | | TGCCTCCATCCGCGAGCTCATCGACCAGCGCCCGCAGTCATCCTTCGAGATTGCATGC |
| | | AAGAATAGCCCCAATGCCACGGTTATCAGCGGCAGCACTGATGAGATTTCTGAGCTCC |
| | | AGTCATCCTTCACGGCATCACGAGCCAGGGCTCTGTCTGTGCCCTATGGATTTCACTC |
| | | CTTCCAGATGGATCCCATGCTCGAGGATTACATCGTTCTTGCGGGTGGTGTAACCTACT |
| | | CGCCACCAAAGATTCCAGTTGCTTCAACCCTGCTCGCTTCGATTGTGGAGTCTTCAGG |
| | | GGTCTTCAACGCTTCCTACCTCGGTCAGCAAACCCGCCAAGCTGTCGACTTCGTCGGT |
| | | GCTCTTGGCGCCTTGAAGGAGAAGTTTGCTGACCCTCTCTGGCTGGAGATCGGACCCA |
| | | GCCAAATCTGCAGCTCCTTTGTCCGGGCGACTCTCTCACCCTCGCCGGGCAAAATCTT |
| | | GTCCACTTTGGAGGCAAATACCAACCCCTGGGCATCCATTTCCAAGTGCCTCGCCGGC |
| | | GCGTACAAGGATGGTGTCGCAGTTGACTGGTTGGCGGTGCATGCTCCATTCAAGGGC |
| | | GGCTTGAAGCTCGTGAAGTTGCCCGCCTATGCATGGGACCTCAAGGACTTCTGGATTG |
| | | TCTACTCTGAGGCCAACAAGGCTGCTCGAGCTTTGGCTCCCGCTCCCTCGTTCGAAAC |
| | | ACAGAGGATTTCTACATGTGCTCAACAGATTGTTGAAGAATCATCATCACCCAGCCTCC |
| | | ATGTCTCTGCCCGAGCTGCTATCTCCGATCCTGGCTTCATGGCCTTGGTCGACGGTCA |
| | | TCGCATGCGCGATGTGTCCATCTGCCCCGGAAGTGTCTTCTGCGAGGCAGGCCTTGC |
| | | CGTCTCCAAGTACGCACTGAAGTACAGTGGCCGAAAGGATACCGTGGAAACAAGACTT |
| | | ACAATCAACAACCTGTCTCTCAAGCGCCCGCTCACAAAGTCTCTTGTAGGCACCGATG |
| | | GCGAGCTTCTCACCACGGTTGTTGCAGACAAGGCCTCCAGCGATACCTTGCAGGTTTC |
| | | ATGGAAGGCTTCTTCCTCTCATGCATCATACGATCTTGGTAGCTGCGAGATCACCATTT |
| | | GTGATGCCCAGACTCTTCAAACTAGCTGGAACAGAAGCTCATACTTCGTCAAGGCTCG |
| | | TATGAACGAGTTGATCAAGAATGTCAAGAGCGGAAATGGTCACCGCATGCTCCCCAGT |
| | | ATCCTCTACACTCTCTTCGCTAGCACAGTTGATTATGACCCTACCTTCAAGTCTGTCAA |
| | | GGAGGCCTTCATCTCAAATGAGTTTGACGAAGCTGCTGCGGAGGTGGTGCTTCAGAAG |
| | | AACCCGGCTGGAACTCAGTTCTTTGCGTCCCCTTACTGGGGTGAGAGCGTAGTTCATC |
| | | TTGCCGGTTTCCTCGTGAACTCCAACCCTGCCCGCAAGACTGCTTCTCAGACGACCTT |
| | | CATGATGCAGAGTCTTGAGAGCGTCGAGCAGACCGCTGATCTCGAGGCTGGACGCAC |
| | | TTACTACACCTATGCTCGCGTTTTGCATGAGGAAGAAGACACAGTCAGCTGTGACTTGT |
| | | TCGTCTTCGACTCGGAGAAGATGGTAATGCAGTGCTCGGGACTCTCATTCCATGAGGT |
| | | CAGCAACAATGTTCTGGACAGACTTCTTGGAAAGGCATCACCGCCTGTGAAGCAAGTT |
| | | TCCCACCAGAAGGCGCCAGTGCTTGTGCCCGCAGAGTCAAAACCGGCCCTGAAAGCT |
| | | GCTGTCGAGGCGGCTCCCAAGGCGCCTGAGCCTGTGAAGACAGAGGTGAAGAAGATC |
| | | TCTTCGTCGGAGAGCGAATTGTTCCACACTATTCTTGAAAGCATCGCCAAGGAGACTG |
| | | GCACTCAGGTCTCTGACTTCACTGATGACATGGAACTGGCTGAACTTGGCGTTGATTC |
| | | CATCATGGGTATTGAGATCGCTGCCGGCGTCAGCAGCAGAACCGGCCTCGATGTTCTC |
| | | CTCCCCTCTTTTGTCGTAGATTATCCCACCATTGGAGATCTGCGAAACGAATTTGCGCG |
| | | CTCCTCTACATCTACACCTCCCAGCAAGACCTTTTCCGAGTTCTCCATCGTCGATGCCA |
| | | CTCCAGAGTCTACGCGCAGCTCGAGTCGAGCGCCTTCTGAGAAGAAGGAGCCTGCTC |
| | | CGGCTTCAGAGAAGTCTGAGGAGCTGGTGATCGTTCCGTCCGCGGTTGTCGAGGATT |
| | | CCTCTCCCCTCCCCAGTGCCAGAATCACCTTGATCCAGGGTCGATCTTCGAGTGGAAA |
| | | GCAGCCTTTCTACTTGATCGCCGATGGAGCTGGTAGCATTGCTACGTATATCCACCTG |
| | | GCTCCCTTCAAGGACAAGAGACCGGTTTATGGCATTGATTCGCCTTTCCTCCGTTGCC |
| | | CCAGCAGGCTGACCACCCAGGTGGGCATTGAAGGCGTCGCAAAGATCATCTTTGAGG |
| | | CGTTGATTAAGTGCCAGCCTGAGGGTCCCTTTGACTTGGGAGGATTCTCTGGCGGAGC |
| | | TATGCTCAGCTATGAGGTGTCTCGCCAACTCGCTGCCGCCGGTCGCGTCGTCTCCAGT |
| | | CTTCTCCTCATCGATATGTGTTCTCCCCGTCCTTTGGGTGTTGAGGACACAATCGAGGT |
| | | CGGCTGGAAGGTCTACGAGACCATCGCTTCCCAAGATAAGCTCTGGAACGCCTCAAGT |
| | | AACACCCAGCAGCATCTCAAGGCCGTCTTCGCCTGCGTCGCAGCCTACCACCCTCCTC |
| | | CCATGACTCCCGCTCAACGACCCAAGCGAACAGCTATCATCTGGGCTAAAAAGGGCAT |
| | | GGTCGACCGTTGTTCTCGCGACGAGAAGGTGATGAAGTTCCTGGCCGACAAGGGCAT |
| | | CCCCACCGAGTCGTACCCAGGGTTCATGGAGGACCCCAAGCTGGGTGCCGTGGCGTG |
| | | GGGCCTTCCGCACAAGTCCGCTGCGGACTTGGGACCCAACGGATGGGACAAGTTCCT |
| | | TGGCGAGACTCTGTGCCTGTCTATCGATTCGGACCACTTGGATATGCCGATGCCGGGG |
| | | CATGTGCACTTGCTTCAGGCGGCGATGGAGGAGTCGTTCAAATATTTCAGCGAGGCAA |
| | | ATTAG |
| 65 | pCHIDT-2.1 | TATCTAAAAATTGCCTTATGATCCGTCTCTCCGGTTACAGCCTGTGTAACTGATTAATCC |
| | | TGCCTTTCTAATCACCATTCTAATGTTTTAATTAAGGGATTTTGTCTTCATTAACGGCTTT |
| | | CGCTCATAAAAATGTTATGACGTTTTGCCCGCAGGCGGGAAACCATCCACTTCACGAG |
| | | ACTGATCTCCTCTGCCGGAACACCGGGCATCTCCAACTTATAAGTTGGAGAAATAAGA |
| | | GAATTTCAGATTGAGAGAATGAAAAAAAAAAAAAAAAAAAAGGCAGAGGAGAGCATAGA |
| | | AATGGGGTTCACTTTTTGGTAAAGCTATAGCATGCCTATCACATATAAATAGAGTGCCA |
| | | GTAGCGACTTTTTTCACACTCGAAATACTCTTACTACTGCTCTCTTGTTGTTTTTATCACT |
| | | TCTTGTTTCTTCTTGGTAAATAGAATATCAAGCTACAAAAAGCATACAATCAACTATCAA |
| | | CTATTAACTATATCGTAATACACAATGCTGGGATTCCCAATGTTCAACCCAGCTACGCC |
| | | TGATGTCTGGAAGATGAATACCCCTTACTTTCCATTTGTTACACCGGGGTTATTTCCTG |
| | | CCTCAGCACCCCCATCGCCCACCAACGTAGATGCCGAAGCTGCCAGTTCCCAACAGTC |
| | | GGAAGCAAGCTATCTGGATAAGGAGAAAATTGTTCGAGGGCCACTTGATTATCTTCTCA |
| | | AATCCCCTGGAAAAGACATTCGTCGGAAATTCATTCACGCGTTCAATGAATGGCTGCGC |
| | | ATTCCTGAGGACAAGTTGAATATTATCACGGAAATTGTTGGATTGCTTCACACGGCCTC |
| | | CCTTCTAATCGACGATATTCAGGACAATTCCAAGCTTCGACGCGGCCTCCCAGTGGCC |
| | | CATAGCATATTTGGTATTGCGCAGACAATTAACTCTGCCAATTATGCGTACTTTCTAGCC |
| | | CAGGAAAGGCTCCGCGAACTGAATCATCCTGAAGCGTACGAAATATACACAGAGGAAC |
| | | TGCTTCGTCTGCACCGCGGTCAAGGTATGGACTTGTACTGGCGGGACTGCCTAACCTG |
| | | TCCCACAGAGGAGGACTATATTGAGATGATCGCCAACAAGACTGGTGGCCTATTTCGA |
| | | CTGGCGATTAAGCTTATGCAGTTGGAAAGCACTTTGTGCAGCAATGTCATTGAACTAGC |
| | | AGACTTGTTGGGCGTGATCTTTCAGATTCGGGATGATTACCAAAACTTACAGAGTGGAC |
| | | TATACGCCAAGAACAAGGGATTTTGCGAGGATTTGACGGAGGGAAAATTTTCCTTTCTG |
| | | ATTATCCACAGTATTAACAGTAACCCGAACAATCACCATCTGCTAAATATACTACGGCA |
| | | GCGGAGCGAGGACGATTCGGTGAAGAAGTATGCTGTTGATTATATCGACTCGACGGG |
| | | GAGTTTTGACTACTGCCGGGAACGGCTCGCTTCCTTATTGGAAGAGGCGGATCAAATG |
| | | GTTAAGAAGTTGGAAAATGAGGGGGGACAATCAAAGGGGATCTACGATATTCTGAGCT |
| | | TTCTGTCGTGAGCGGATCTCTTATGTCTTTACGATTTATAGTTTTCATTATCAAGTATGC |
| | | CTATATTAGTATATAGCATCTTTAGATGACAGTGTTCGAAGTTTCACGAATAAAAGATAA |
| | | TATTCTACTTTTTGCTCCCACCGCGTTTGCTAGCACGAGTGAACACCATCCCTCGCCTG |
| | | TGAGTTGTACCCATTCCTCTAAACTGTAGACATGGTAGCTTCAGCAGTGTTCGTTATGT |
| | | ACGGCATCCTCCAACAAACAGTCGGTTATAGTTTGTCCTGCTCCTCTGAATCGTCTCCC |
| | | TCGATATTTCTCATTTTCCTTCGCATGCCAGCATTGAAATGATCGAAGTTCAATGATGAA |
| | | ACGGTAATTCTTCTGTCATTTACTCATCTCATCTCATCAAGTTATATAATTCTATACGGAT |
| | | GTAATTTTTCACTTTTCGTCTTGACGTCCACCCTATAATTTCAATTATTGAACCCTCACG |
| | | ATCCAGTTCTCCAGTGACACAGCCTTTATCTGGTCAAACCTTTCTTTCTAATCACCTATG |
| | | CTGATGCTTAATTAAGGGATTTTTGTCTCCATCAACGGCATGCGCCCAAAAATGACGTT |
| | | TTTTTTAACCCATAGACACGAAACTACCCATTTTCCACCGGCCTGACCTACCACCGGAA |
| | | CAACGGCCATCTCCAACTTGCAAGTTGGGGAAATTAAGAGCATCGCAGGTTTAATGGA |
| | | AGAAAAAAAAAAGGTACAGCACAGCGCAAATGGAGTTAGTTCCCTTATGTCACACACTC |
| | | ACACACAGTCGGTCAGATCAAGCATACTGGGTGCGTATAAATAGAGTGGCCATTGCCA |
| | | CCCTGTTTATCTCAAAATCTGTCTTGTTAGTGGTCTTCTCCCTTTTTCAGGTTACAATTCT |
| | | CTTGTTTCTACTTAGTATATAAGTATATCAAGCTATATTAAGCATACTATCAACTGTCAAC |
| | | TCTATCCTCAAAATACAATACAAAATGGATGGGTTCGACCATTCTACTGCTCCACCAGG |
| | | ATATAACGAGCTAAAATGGCTCGCCGATATCTTCGTCATCGGAATGGCTGTTGGCTGG |
| | | GTTGCTCACTATATGGAGATGATTCACACGTCGTTCAAGGACCAAACATACTGCATGAC |
| | | CATCGGGGGCCTTTGCATCAATTTTGCCTGGGAAATCATATTCTGCACAATGTATCCTG |
| | | CCAAAGGATTTGTCGAGCGGGTTGCCTTTCTCATGGGCATTTCTCTCGACCTTGGGGT |
| | | TATTTACGCGGGAATCAAGAACGCCCCAAATGAATGGCACCACTCTGCAATGGTGAGG |
| | | GACCATATGCCCCTTGTCTTCGCAGCAACGACACTTTGTTGTCTGAGCGGTCATATGG |
| | | CTCTTACTGCCCAGGTTGGTCCCGCACAAGCCTATACGTGGGGGGCAATTGCATGCCA |
| | | GCTCTTTATCAGCATAGGGAATGTGTTTCAATTGTTGAGTCGGGGAAACACACGAGGG |
| | | GCGTCATGGACGCTATGGACCTCCAGGTTTTTTGGATCAACATCAGCCATTGGCTTTGC |
| | | TCTTGTTCGATATATTCGCTGGTGGGAGGCCTTTTCTTGGTTGAACTGCCCGCTTGTGA |
| | | TATGGTCCGTGGCCATGTTCTTTCTGTTTGAAACACTCTATGGAGCCCTATTCTATTCTG |
| | | TCAAGCGACAAGAAGGGAGATCCCAGCGTGGAATCAAGCACAAAGAGAGGTAGACAA |
| | | ATCGCTCTTAAATATATACCTAAAGAACATTAAAGCTATATTATAAGCAAAGATACGTAA |
| | | ATTTTGCTTATATTATTATACACATATCATATTTCTATATTTTTAAGATTTGGTTATATAAT |
| | | GTACGTAATGCAAAGGAAATAAATTTTATACATTATTGAACAGCGTCCAAGTAACTACAT |
| | | TATGTGCACTAATAGTTTAGCGTCGTGAAGACTTTATTGTGTCGCGAAAAGTAAAAATTT |
| | | TAAAAATTAGAGCACCTTGAACTTGCGAAAAAGGTTCTCATCAACTGTTTAAAAGGAGG |
| | | ATATCAGGTCCTATTTCTGACAAACAATATACAAATTTAGTTTCAAAGATGAATCAGTGC |
| | | GCGAAGGACATAACTCAATAGGAAAAAACCGAGCTTCCTTTCATCCGGCGCGGCTGTG |
| | | TTCTACATATCACTGAAGCTCCGGGTATTTTAAGTTATACAAGGGAAAGATGCCGGCTA |
| | | GACTAGCAAGTTTTAGGCTGCTTAACATTATGGATAGGCGGATAAAGGGCCCAAACAG |
| | | GATTGTAAAGCTTAGACGCTTCTGGTTGGACAATGGTACGTTTGTGTATTAAGTAAGGC |
| | | TTGGCTGGGGATAGCAACATTGGGCAGAGTATAGAAGACCACAAAAAAAAGGTATATA |
| | | AGGGCAGAGAAGTCTTTGTTAATGTGTGTAACTTCTCTTCCATGTGTAATCAGTATTTCTA |
| | | CTTACTTCTTAAATATACAGAAGTAAGACAGATAACCAACAGCCTTTCCCAGATATACAT |
| | | ATATATCTTTATTTCAGCTTAAACAATAATTATATTTGTTTAACTCAAAAATAAAAAAAAAA |
| | | AACCAAACTCACGCAACTAATTATTCCATAATAAAATAACAACATGGCGGCACTTCCGG |
| | | ACGTTGCCTCCATTCCCATCCCTCTGGTGGCAACCCTAGGCATTGCCCCTCTAATTTTC |
| | | TATCTCGTCCTTGATAGAATTAGCCCCTTGTGGCCAAATTCCAAAGCTTTCCTGATTGG |
| | | CAAGAAGAAACCGGAGACCGTGACATCGTTCGAGTGCCCATATGCCTACATCCGTCAG |
| | | ATCTATGGGAAGTATCACTGGGAGCCATTCGTACAGAAGCTGTCTCCGAGGCTTAAGG |
| | | ATGAGGATCCGGCCAAATATAAGATGGTTCTGGAGATAATGGATGCAATCCACCTGTGT |
| | | CTGATGCTAGTTGACGATATAACTGACAATAGCGACTATCGAAAAGGCAAGCCAGCAG |
| | | CCCACCGGATATATGGCCCTTCAGAGACAGCAAATCGCGCTTACTACCGAGTCACCCA |
| | | GATTCTAAACAAGACCGTGCAAAAGTTCCCCAAGCTGGCCAAGTTCCTGCTTCAGAATC |
| | | TGGAAGAAATTCTCGAAGGCCAAGACCTGTCAGTAATCTGGCGACGGGATGGAGTGGG |
| | | TAGCCTTTCGACTGTTCCTGATGAGCGAGTTGCAGCCTATCGCAAGATGGCGTCATTG |
| | | AAAACTGGGGCGTTATTCCGGGTGCTGGGGCAATTGGTGATGGAGGACCAATCGATG |
| | | GACGGGACGATGACTACTCTTGCGTGGTGCTCTCAGCTGCAGAATGACTGCAAGAATG |
| | | TCTACTCATCTGAATATGCTAAGGCCAAAGGGGCGCTTGCCGAAGACCTCCGAAATCG |
| | | AGAGCTCTCATTTCCAATTATCCTCGCGCTGGAAGCTCCTGAAGGGCATTGGGTCGCC |
| | | AGTGCTTTGGAGACCAGCTCACCGCGCAACATTCGCAAGGCGCTTGCTGTGATTCAGA |
| | | GTGAGAGAGTGCGCAATGCTTGTTTCAAGGAGCTCAAGTCGGCGAGTGCTTCGGTCCA |
| | | GGACTGGTTGGCTATTTGGGGACGGAACGAGAAAATGAACTTGAAGAGCCAGCAGAC |
| | | GTAGAGTGCTTTTAACTAAGAATTATTAGTCTTTTCTGCTTATTTTTTCATCATAGTTTAG |
| | | AACACTTTATATTAACGAATAGTTTATGAATCTATTTAGGTTTAAAAATTGATACAGTTTT |
| | | ATAAGTTACTTTTTCAAAGACTCGTGCTGTCTATTGCATAATGCACTGGAAGGGGAAAA |
| | | AAAAGGTGCACACGCGTGGCTTTTTCTTGAATTTGCAGTTTGAAAAATAACTACATGGA |
| | | TGATAAGAAAACATGGAGTACAGTCACTTTGAGAACCTTCAATCAGCTGGTAACGTCTT |
| | | CGTTAATTGGATACTCAAAAAAGATGGATAGCATGAATCACAAGATGGAAGGAAATGCG |
| | | GGCCACGACCACAGTGATATGCATATGGGAGATGGAGATGATACCTCCATTGGGCCGA |
| | | TGAAGTTAGTCGACGGATAGAAGCGGTTGTCCCCTTTCCCGGCGAGCCGGCAGTCGG |
| | | GCCGAGGTTCGGATAAATTTTGTATTGTGTTTTGATTCTGTCATGAGTATTACTTATGTT |
| | | CTCTTTAGGTAACCCCAGGTTAATCAATCACAGTTTCATACCGGCTAGTATTCAAATTAT |
| | | GACTTTTCTTCTGCAGTGTCAGCCTTACGACGATTATCTATGAGCTTTGAATATAGTTTG |
| | | CCGTGATTCGTATCTTTAATTGGATAATAAAATGCGAAGGATCGATGACCCTTATTATTA |
| | | TTTTTCTACACTGGCTACCGATTTAACTCATCTTCTTGAAAGTATATAAGTAACAGTAAAA |
| | | TATACCGTACTTCTGCTAATGTTATTTGTCCCTTATTTTTCTTTTCTTGTCTTATGCTATA |
| | | GTACCTAAGAATAACGACTATTGTTTTGAACTAAACAAAGTAGTAAAAGCACATAAAAGA |
| | | ATTAAGAAAATGGCCAATGCCCAGCAACCCCCCGTTTCGCATCCTTATTGTGGGCGGTTC |
| | | TGTCGCAGGCCTCATCCTTGCGCACTGTCTCGAACGCGCCAATATAGAGTACCTCATA |
| | | CTCGAAAAAGGAGAAGATGTTGCTCCACAAGTTGGTGCGTCGATAGGTATCATGCCAA |
| | | ATGGCGGACGGATCCTCGAGCAACTGGGCCTATTTGGGGAGATTGAGCGTGTGATCG |
| | | AGCCGTTGCATCAGGCGAATATCAGCTATCCAGATGGGTTCTGCTTTAGTAACGTCTAT |
| | | CCTAAGGTTCTTGGCGACAGGTTCGGATACCCGGTTGCATTCTTGGACCGGCAGAAGT |
| | | TCCTGCAGATTGCATATGAGGGGCTGAGAAAGAAGCAGAATGTTCTCACCGGTAAAAG |
| | | GGTAGTTGGACTGCGACAGTCGGATCAAGGGACTGCTGTTTCTGTGGCTGACGGGAC |
| | | AGAGTATGAGGCGGATCTCGTGGTTGGTGCTGATGGAGTACATAGTCGGGTGAGAAGT |
| | | GAGATTTGGAAGATGGCGGAAGAGAATCAGCCTGCATCAGTTTCGACACGTGAAAGAA |
| | | GAAGCATGACTGTTGAATATGTCTGCGTTTTCGGGATTTCATCAGCCATCCCAGGGCTC |
| | | GAGATAAGCGAACAGATCAACGGTATTTTCGACCATCTATCCATTCTAACAATCCATGG |
| | | CAGACATGGTCGCGTGTTCTGGTTCGTGATCCAGAAGCTGGATAGGAAGTACGTCTAT |
| | | CCTGATGTCCCGCGATTCTCAGACGAGGATGCCGTACAGCTCTTCGATCGGGTCAAAC |
| | | ACGTGCGGTTCTGGAAAAACATCTGTGTGGGGGACTTGTGGAAGAACAGAGAGGTGTC |
| | | CTCGATGACAGCGCTGGAGGAGGGAGTGTTCGAGACATGGCATCATGATAGGATGGT |
| | | TTTGATTGGAGATAGCGTTCACAAGATGACGCCCAACTTTGGCCAAGGAGCTAATTCAG |
| | | CCATCGAGGATGCTGCCGCGCTCTCTTCCCTTCTACATGATCTCGTCAACGCCCGTGG |
| | | AGTTTGCAAGCCATCGAATGTCCAGATTCAGCATCTCCTCAAGCAGTATCGGGAGACC |
| | | CGATACACTCGCATGGTAGGCATGTGTCGCACCGCGGCTTCAGTCTCTCGGATTCAGG |
| | | CCCGAGATGGCATCCTCAACACCGTCTTTGGACGATATTGGGCACCTTATGCTGGCAA |
| | | CCTGCCTGCTGACCTGGCATCAAAAGTGATGGCAGATGCAGAGGTTGTTACTTTTCTG |
| | | CCCTTGCCAGGGCGCTCAGGACCGGGCTGGGAGATGTACAGACGAAAGGGGAAGGG |
| | | AGGGCAGGTGCAATGGGTGCTTATAATCTTAAGCTTACTTACGATTGGTGGATTGTGCA |
| | | TCTGGCTACAAAGCAATGCGTTGAGTAGATAAGGAGATTGATAAGACTTTTCTAGTTGC |
| | | ATATCTTTTATATTTAAATCTTATCTATTAGTTAATTTTTTGTAATTTATCCTTATATATAGT |
| | | CTGGTTATTCTAAAATATCATTTCAGTATCTAAAAATTCCCCTCTTTTTTCAGTTATATCTT |
| | | AACAGGCGACAGTCCAAATGTTGATTTATCCCAGTCCGATTCATCAGGGTTGTGAAGCA |
| | | TTTTGTCAATGGTCGAAATCACATCAGTAATAGTGCCTCTTACTTGCCTCATAGAATTTC |
| | | TTTCTCTTAACGTCACCGTTTGGTCTTTTATAGTTTCGAAATCTATGGTGATACCAAATG |
| | | GTGTTCCCAATTCATCGTTACGGGCGTATTTTTTACCAATTGAAGTATTGGAATCGTCAA |
| | | TTTTAAAGTATATCTCTCTTTTACGTAAAGCCTGCGAGATCCTCTTAAGTATAGCGGGGA |
| | | AGCCATCGTTATTCGATATTGTCGTAACAAATACTTTGATCGGCGCTATGCGGCCGCCA |
| | | CCGCGGTGGAGCTCCAGCTTTTGTTCCCTTTAGTGAGGGTTAATTGCGCGCTTGGCGT |
| | | AATCATGGTCATAGCTGTTTCCTGTGTGAAATTGTTATCCGCTCACAATTCCACACAACA |
| | | TAGGAGCCGGAAGCATAAAGTGTAAAGCCTGGGGTGCCTAATGAGTGAGGTAACTCAC |
| | | ATTAATTGCGTTGCGCTCACTGCCCGCTTTCCAGTCGGGAAACCTGTCGTGCCAGCTG |
| | | CATTAATGAATCGGCCAACGCGCGGGGAGAGGCGGTTTGCGTATTGGGCGCTCTTCC |
| | | GCTTCCTCGCTCACTGACTCGCTGCGGTCGGTCGTTCGGCTGCGGCGAGCGGTATCA |
| | | GCTCACTCAAAGGCGGTAATACGGTTATCCACAGAATCAGGGGATAACGCAGGAAAGA |
| | | ACATGTGAGCAAAAGGCCAGCAAAAGGCCAGGAACCGTAAAAAGGCCGCGTTGCTGG |
| | | CGTTTTTCCATAGGCTCCGCCCCCCTGACGAGCATCACAAAAATCGACGCTCAAGTCA |
| | | GAGGTGGCGAAACCCGACAGGACTATAAAGATACCAGGCGTTTCCCCCTGGAAGCTC |
| | | CCTCGTGCGCTCTCCTGTTCCGACCCTGCCGCTTACCGGATACCTGTCCGCCTTTCTC |
| | | CCTTCGGGAAGCGTGGCGCTTTCTCATAGCTCACGCTGTAGGTATCTCAGTTCGGTGT |
| | | AGGTCGTTCGCTCCAAGCTGGGCTGTGTGCACGAACCCCCCGTTCAGCCCGACCGCT |
| | | GCGCCTTATCCGGTAACTATCGTCTTGAGTCCAACCCGGTAAGACACGACTTATCGCC |
| | | ACTGGCAGCAGCCACTGGTAACAGGATTAGCAGAGCGAGGTATGTAGGCGGTGCTAC |
| | | AGAGTTCTTGAAGTGGTGGCCTAACTACGGCTACACTAGAAGGACAGTATTTGGTATCT |
| | | GCGCTCTGCTGAAGCCAGTTACCTTCGGAAAAAGAGTTGGTAGCTCTTGATCCGGCAA |
| | | ACAAACCACCGCTGGTAGCGGTGGTTTTTTTGTTTGCAAGCAGCAGATTACGCGCAGA |
| | | AAAAAAGGATCTCAAGAAGATCCTTTGATCTTTTCTACGGGGTCTGACGCTCAGTGGAA |
| | | CGAAAACTCACGTTAAGGGATTTTGGTCATGAGATTATCAAAAAGGATCTTCACCTAGA |
| | | TCCTTTTAAATTAAAAATGAAGTTTTAAATCAATCTAAAGTATATATGAGTAAACTTGGTC |
| | | TGACAGTTACCAATGCTTAATCAGTGAGGCACCTATCTCAGCGATCTGTCTATTTCGTT |
| | | CATCCATAGTTGCCTGACTCCCCGTCGTGTAGATAACTACGATACGGGAGGGCTTACC |
| | | ATCTGGCCCCAGTGCTGCAATGATACCGCGAGACCCACGCTCACCGGCTCCAGATTTA |
| | | TCAGCAATAAACCAGCCAGCCGGAAGGGCCGAGCGCAGAAGTGGTCCTGCAACTTTAT |
| | | CCGCCTCCATCCAGTCTATTAATTGTTGCCGGGAAGCTAGAGTAAGTAGTTCGCCAGTT |
| | | AATAGTTTGCGCAACGTTGTTGCCATTGCTACAGGCATCGTGGTGTCACGCTCGTCGTT |
| | | TGGTATGGCTTCATTCAGCTCCGGTTCCCAACGATCAAGGCGAGTTACATGATCCCCC |
| | | ATGTTGTGCAAAAAAGCGGTTAGCTCCTTCGGTCCTCCGATCGTTGTCAGAAGTAAGTT |
| | | GGCCGCAGTGTTATCACTCATGGTTATGGCAGCACTGCATAATTCTCTTACTGTCATGC |
| | | CATCCGTAAGATGCTTTTCTGTGACTGGTGAGTACTCAACCAAGTCATTCTGAGAATAG |
| | | TGTATGCGGCGACCGAGTTGCTCTTGCCCGGCGTCAATACGGGATAATACCGCGCCA |
| | | CATAGCAGAACTTTAAAAGTGCTCATCATTGGAAAACGTTCTTCGGGGCGAAAACTCTC |
| | | AAGGATCTTACCGCTGTTGAGATCCAGTTCGATGTAACCCACTCGTGCACCCAACTGAT |
| | | CTTCAGCATCTTTTACTTTCACCAGCGTTTCTGGGTGAGCAAAAACAGGAAGGCAAAAT |
| | | GCCGCAAAAAAGGGAATAAGGGCGACACGGAAATGTTGAATACTCATACTCTTCCTTTT |
| | | TCAATATTATTGAAGCATTTATCAGGGTTATTGTCTCATGAGCGGATACATATTTGAATG |
| | | TATTTAGAAAAATAAACAAATAGGGGTTCCGCGCACATTTCCCCGAAAAGTGCCACCTG |
| | | AACGAAGCATCTGTGCTTCATTTTGTAGAACAAAAATGCAACGCGAGAGCGCTAATTTT |
| | | TCAAACAAAGAATCTGAGCTGCATTTTTACAGAACAGAAATGCAACGCGAAAGCGCTAT |
| | | TTTACCAACGAAGAATCTGTGCTTCATTTTTGTAAAACAAAAATGCAACGCGAGAGCGC |
| | | TAATTTTTCAAACAAAGAATCTGAGCTGCATTTTTACAGAACAGAAATGCAACGCGAGA |
| | | GCGCTATTTTACCAACAAAGAATCTATACTTCTTTTTTGTTCTACAAAAATGCATCCCGA |
| | | GAGCGCTATTTTTCTAACAAAGCATCTTAGATTACTTTTTTTCTCCTTTGTGCGCTCTATA |
| | | ATGCAGTCTCTTGATAACTTTTTGCACTGTAGGTCCGTTAAGGTTAGAAGAAGGCTACT |
| | | TTGGTGTCTATTTTCTCTTCCATAAAAAAAGCCTGACTCCACTTCCCGCGTTTACTGATT |
| | | ACTAGCGAAGCTGCGGGTGCATTTTTTCAAGATAAAGGCATCCCCGATTATATTCTATA |
| | | CCGATGTGGATTGCGCATACTTTGTGAACAGAAAGTGATAGCGTTGATGATTCTTCATT |
| | | GGTCAGAAAATTATGAACGGTTTCTTCTATTTTGTCTCTATATACTACGTATAGGAAATG |
| | | TTTACATTTTCGTATTGTTTTCGATTCACTCTATGAATAGTTCTTACTACAATTTTTTTGTC |
| | | TAAAGAGTAATACTAGAGATAAACATAAAAAATGTAGAGGTCGAGTTTAGATGCAAGTT |
| | | CAAGGAGCGAAAGGTGGATGGGTAGGTTATATAGGGATATAGCACAGAGATATATAGC |
| | | AAAGAGATACTTTTGAGCAATGTTTGTGGAAGCGGTATTCGCAATATTTTAGTAGCTCG |
| | | TTACAGTCCGGTGCGTTTTTGGTTTTTTGAAAGTGCGTCTTCAGAGCGCTTTTGGTTTTC |
| | | AAAAGCGCTCTGAAGTTCCTATACTTTCTAGAGAATAGGAACTTCGGAATAGGAACTTC |
| | | AAAGCGTTTCCGAAAACGAGCGCTTCCGAAAATGCAACGCGAGCTGCGCACATACAGC |
| | | TCACTGTTCACGTCGCACCTATATCTGCGTGTTGCCTGTATATATATATACATGAGAAGA |
| | | ACGGCATAGTGCGTGTTTATGCTTAAATGCGTACTTATATGCGTCTATTTATGTAGGATG |
| | | AAAGGTAGTCTAGTACCTCCTGTGATATTATCCCATTCCATGCGGGGTATCGTATGCTT |
| | | CCTTCAGCACTACCCTTTAGCTGTTCTATATGCTGCCACTCCTCAATTGGATTAGTCTCA |
| | | TCCTTCAATGCTATCATTTCCTTTGATATTGGATCATACTAAGAAACCATTATTATCATGA |
| | | CATTAACCTATAAAAATAGGCGTATCACGAGGCCCTTTCGTCTCGCGCGTTTCGGTGAT |
| | | GACGGTGAAAACCTCTGACACATGCAGCTCCCGGAGACGGTCACAGCTTGTCTGTAAG |
| | | CGGATGCCGGGAGCAGACAAGCCCGTCAGGGCGCGTCAGCGGGTGTTGGCGGGTGT |
| | | CGGGGCTGGCTTAACTATGCGGCATCAGAGCAGATTGTACTGAGAGTGCACCATATCG |
| | | ACTACGTCGTAAGGCCGTTTCTGACAGAGTAAAATTCTTGAGGGAACTTTCACCATTAT |
| | | GGGAAATGCTTCAAGAAGGTATTGACTTAAACTCCATCAAATGGTCAGGTCATTGAGTG |
| | | TTTTTTATTTGTTGTATTTTTTTTTTTTTAGAGAAAATCCTCCAATATCAAATTAGGAATCG |
| | | TAGTTTCATGATTTTCTGTTACACCTAACTTTTTGTGTGGTGCCCTCCTCCTTGTCAATA |
| | | TTAATGTTAAAGTGCAATTCTTTTTCCTTATCACGTTGAGCCATTAGTATCAATTTGCTTA |
| | | CCTGTATTCCTTTACTATCCTCCTTTTTCTCCTTCTTGATAAATGTATGTAGATTGCGTAT |
| | | ATAGTTTCGTCTACCCTATGAACATATTCCATTTTGTAATTTCGTGTCGTTTCTATTATGA |
| | | ATTTCATTTATAAAGTTTATGTACAAATATCATAAAAAAAGAGAATCTTTTTAAGCAAGGA |
| | | TTTTCTTAACTTCTTCGGCGACAGCATCACCGACTTCGGTGGTACTGTTGGAACCACCT |
| | | AAATCACCAGTTCTGATACCTGCATCCAAAACCTTTTTAACTGCATCTTCAATGGCCTTA |
| | | CCTTCTTCAGGCAAGTTCAATGACAATTTCAACATCATTGCAGCAGACAAGATAGTGGC |
| | | GATAGGGTCAACCTTATTCTTTGGCAAATCTGGAGCAGAACCGTGGCATGGTTCGTAC |
| | | AAACCAAATGCGGTGTTCTTGTCTGGCAAAGAGGCCAAGGACGCAGATGGCAACAAAC |
| | | CCAAGGAACCTGGGATAACGGAGGCTTCATCGGAGATGATATCACCAAACATGTTGCT |
| | | GGTGATTATAATACCATTTAGGTGGGTTGGGTTCTTAACTAGGATCATGGCGGCAGAAT |
| | | CAATCAATTGATGTTGAACCTTCAATGTAGGGAATTCGTTCTTGATGGTTTCCTCCACAG |
| | | TTTTTCTCCATAATCTTGAAGAGGCCAAAAGATTAGCTTTATCCAAGGACCAAATAGGCA |
| | | ATGGTGGCTCATGTTGTAGGGCCATGAAAGCGGCCATTCTTGTGATTCTTTGCACTTCT |
| | | GGAACGGTGTATTGTTCACTATCCCAAGCCACACCATCACCATCGTCTTCCTTTCTCTT |
| | | ACCAAAGTAAATACCTCCCACTAATTCTCTGACAACAACGAAGTCAGTACCTTTAGCAA |
| | | ATTGTGGCTTGATTGGAGATAAGTCTAAAAGAGAGTCGGATGCAAAGTTACATGGTCTT |
| | | AAGTTGGCGTACAATTGAAGTTCTTTACGGATTTTTAGTAAACCTTGTTCAGGTCTAACA |
| | | CTACCGGTACCCCATTTAGGACCAGCCACAGCACCTAACAAAACGGCATCAACCTTCTT |
| | | GGAGGCTTCCAGCGCCTCATCTGGAAGTGGGACACCTGTAGCATCGATAGCAGCACC |
| | | ACCAATTAAATGATTTTCGAAATCGAACTTGACATTGGAACGAACATCAGAAATAGCTTT |
| | | AAGAACCTTAATGGCTTCGGCTGTGATTTCTTGACCAACGTGGTCACCTGGCAAAACGA |
| | | CGATCTTCTTAGGGGCAGACATAGGGGCAGACATTAGAATGGTATATCCTTGAAATATA |
| | | TATATATATTGCTGAAATGTAAAAGGTAAGAAAAGTTAGAAAGTAAGACGATTGCTAACC |
| | | ACCTATTGGAAAAAACAATAGGTCCTTAAATAATATTGTCAACTTCAAGTATTGTGATGC |
| | | AAGCATTTAGTCATGAACGCTTCTCTATTCTATATGAAAAGCCGGTTCCGGCCTCTCAC |
| | | CTTTCCTTTTTCTCCCAATTTTTCAGTTGAAAAAGGTATATGCGTCAGGCGACCTCTGAA |
| | | ATTAACAAAAAATTTCCAGTCATCGAATTTGATTCTGTGCGATAGCGCCCCTGTGTGTTC |
| | | TCGTTATGTTGAGGAAAAAAATAATGGTTGCTAAGAGATTCGAACTCTTGCATCTTACGA |
| | | TACCTGAGTATTCCCACAGTTAACTGCGGTCAAGATATTTCTTGAATCAGGCGCCTTAG |
| | | ACCGCTCGGCCAAACAACCAATTACTTGTTGAGAAATAGAGTATAATTATCCTATAAATA |
| | | TAACGTTTTTGAACACACATGAACAAGGAAGTACAGGACAATTGATTTTGAAGAGAATG |
| | | TGGATTTTGATGTAATTGTTGGGATTCCATTTTTAATAAGGCAATAATATTAGGTATGTG |
| | | GATATACTAGAAGTTCTCCTCGACCGTCGATATGCGGTGTGAAATACCGCACAGATGC |
| | | GTAAGGAGAAAATACCGCATCAGGAAATTGTAAACGTTAATATTTTGTTAAAATTCGCGT |
| | | TAAATTTTTGTTAAATCAGCTCATTTTTTAACCAATAGGCCGAAATCGGCAAAATCCCTT |
| | | ATAAATCAAAAGAATAGACCGAGATAGGGTTGAGTGTTGTTCCAGTTTGGAACAAGAGT |
| | | CCACTATTAAAGAACGTGGACTCCAACGTCAAAGGGCGAAAAACCGTCTATCAGGGCG |
| | | ATGGCCCACTACGTGAACCATCACCCTAATCAAGTTTTTTGGGGTCGAGGTGCCGTAA |
| | | AGCACTAAATCGGAACCCTAAAGGGAGCCCCCGATTTAGAGCTTGACGGGGAAAGCC |
| | | GGCGAACGTGGCGAGAAAGGAAGGGAAGAAAGCGAAAGGAGCGGGCGCTAGGGCGC |
| | | TGGCAAGTGTAGCGGTCACGCTGCGCGTAACCACCACACCCGCCGCGCTTAATGCGC |
| | | CGCTACAGGGCGCGTCGCGCCATTCGCCATTCAGGCTGCGCAACTGTTGGGAAGGGC |
| | | GATCGGTGCGGGCCTCTTCGCTATTACGCCAGCTGGCGAAAGGGGGATGTGCTGCAA |
| | | GGCGATTAAGTTGGGTAACGCCAGGGTTTTCCCAGTCACGACGTTGTAAAACGACGGC |
| | | CAGTGAGCGCGCGTAATACGACTCACTATAGGGCGAATTGGGTACCGGGCCCCCCCT |
| | | CGAGGTCGACGGTATCGATAAGCTTGATATCGAATTCCTGCAGCCCGGGGGATCCACT |
| | | AGTTCTAGATTAATTAA |
| 66 | pCHIDT-2c | ATAGCTTCAAAATGTTTCTACTCCTTTTTTACTCTTCCAGATTTTCTCGGACTCCGCGCA |
| | | TCGCCGTACCACTTCAAAACACCCAAGCACAGCATACTAAATTTCCCCTCTTTCTTCCT |
| | | CTAGGGTGTCGTTAATTACCCGTACTAAAGGTTTGGAAAAGAAAAAAGAGACCGCCTC |
| | | GTTTCTTTTTCTTCGTCGAAAAAGGCAATAAAAATTTTTATCACGTTTCTTTTTCTTGAAA |
| | | ATTTTTTTTTTTGATTTTTTTCTCTTTCGATGACCTCCCATTGATATTTAAGTTAATAAACG |
| | | GTCTTCAATTTCTCAAGTTTCAGTTTCATTTTTCTTGTTCTATTACAACTTTTTTTACTTCT |
| | | TGCTCATTAGAAAGAAAGCATAGCAATCTAATCTAAGTTTTAATTACAAAATGCTGGGAT |
| | | TCCCAATGTTCAACCCAGCTACGCCTGATGTCTGGAAGATGAATACCCCTTACTTTCCA |
| | | TTTGTTACACCGGGGTTATTTCCTGCCTCAGCACCCCCATCGCCCACCAACGTAGATG |
| | | CCGAAGCTGCCAGTTCCCAACAGTCGGAAGCAAGCTATCTGGATAAGGAGAAAATTGT |
| | | TCGAGGGCCACTTGATTATCTTCTCAAATCCCCTGGAAAAGACATTCGTCGGAAATTCA |
| | | TTCACGCGTTCAATGAATGGCTGCGCATTCCTGAGGACAAGTTGAATATTATCACGGAA |
| | | ATTGTTGGATTGCTTCACACGGCCTCCCTTCTAATCGACGATATTCAGGACAATTCCAA |
| | | GCTTCGACGCGGCCTCCCAGTGGCCCATAGCATATTTGGTATTGCGCAGACAATTAAC |
| | | TCTGCCAATTATGCGTACTTTCTAGCCCAGGAAAGGCTCCGCGAACTGAATCATCCTGA |
| | | AGCGTACGAAATATACACAGAGGAACTGCTTCGTCTGCACCGCGGTCAAGGTATGGAC |
| | | TTGTACTGGCGGGACTGCCTAACCTGTCCCACAGAGGAGGACTATATTGAGATGATCG |
| | | CCAACAAGACTGGTGGCCTATTTCGACTGGCGATTAAGCTTATGCAGTTGGAAAGCAC |
| | | TTTGTGCAGCAATGTCATTGAACTAGCAGACTTGTTGGGCGTGATCTTTTAGATTCGGG |
| | | ATGATTACCAAAACTTACAGAGTGGACTATACGCCAAGAACAAGGGATTTTGCGAGGAT |
| | | TTGACGGAGGGAAAATTTTCCTTTCTGATTATCCACAGTATTAACAGTAACCCGAACAAT |
| | | CACCATCTGCTAAATATACTACGGCAGCGGAGCGAGGACGATTCGGTGAAGAAGTATG |
| | | CTGTTGATTATATCGACTCGACGGGGAGTTTTGACTACTGCCGGGAACGGCTCGCTTC |
| | | CTTATTGGAAGAGGCGGATCAAATGGTTAAGAAGTTGGAAAATGAGGGGGGACAATCA |
| | | AAGGGGATCTACGATATTCTGAGCTTTCTGTCGTGAGCGGATCTCTTATGTCTTTACGA |
| | | TTTATAGTTTTCATTATCAAGTATGCCTATATTAGTATATAGCATCTTTAGATGACAGTGT |
| | | TCGAAGTTTCACGAATAAAAGATAATATTCTACTTTTTGCTCCCACCGCGTTTGCTAGCA |
| | | CGAGTGAACACCATCCCTCGCCTGTGAGTTGTACCCATTCCTCTAAACTGTAGACATGG |
| | | TAGCTTCAGCAGTGTTCGTTATGTACGGCATCCTCCAACAAACAGTCGGTTATAGTTTG |
| | | TCCTGCTCCTCTGAATCGTCTCCCTCGATATTTCTCATTTTCCTTCGCATGCCAGCATTG |
| | | AAATGATCGAAGTTCAATGATGAAACGGTAATTCTTCTGTCATTTACTCATCTCATCTCA |
| | | TCAAGTTATATAATTCTATACGGATGTAATTTTTCACTTTTCGTCTTGACGTCCACCCTAT |
| | | AATTTCAATTATTGAACCCTCACTGGGTCATTACGTAAATAATGATAGGAATGGGATTCT |
| | | TCTATTTTTCCTTTTTCCATTCTAGCAGCCGTCGGGAAAACGTGGCATCCTCTCTTTCG |
| | | GGCTCAATTGGAGTCACGCTGCCGTGAGCATCCTCTCTTTCCATATCTAACAACTGAGC |
| | | ACGTAACCAATGGAAAAGCATGAGCTTAGCGTTGCTCCAAAAAAGTATTGGATGGTTAA |
| | | TACCATTTGTCTGTTCTCTTCTGACTTTGACTCCTCAAAAAAAAAAAATCTACAATCAACA |
| | | GATCGCTTCAATTACGCCCTCACAAAAACTTTTTTCCTTCTTCTTCGCCCACGTTAAATT |
| | | TTATCCCTCATGTTGTCTAACGGATTTCTGCACTTGATTTATTATAAAAAGACAAAGACA |
| | | TAATACTTCTCTATCAATTTCAGTTATTGTTCTTCCTTGCGTTATTCTTCTGTTCTTCTTTT |
| | | TCTTTTGTCATATATAACCATAACCAAGTAATACATATTCAAAATGGATGGGTTCGACCA |
| | | TTCTACTGCTCCACCAGGATATAACGAGCTAAAATGGCTCGCCGATATCTTCGTCATCG |
| | | GAATGGCTGTTGGCTGGGTTGCTCACTATATGGAGATGATTCACACGTCGTTCAAGGA |
| | | CCAAACATACTGCATGACCATCGGGGGCCTTTGCATCAATTTTGCCTGGGAAATCATAT |
| | | TCTGCACAATGTATCCTGCCAAAGGATTTGTCGAGCGGGTTGCCTTTCTCATGGGCATT |
| | | TCTCTCGACCTTGGGGTTATTTACGCGGGAATCAAGAACGCCCCAAATGAATGGCACC |
| | | ACTCTGCAATGGTGAGGGACCATATGCCCCTTGTCTTCGCAGCAACGACACTTTGTTGT |
| | | CTGAGCGGTCATATGGCTCTTACTGCCCAGGTTGGTCCCGCACAAGCCTATACGTGGG |
| | | GGGCAATTGCATGCCAGCTCTTTATCAGCATAGGGAATGTGTTTCAATTGTTGAGTCGG |
| | | GGAAACACACGAGGGGCGTCATGGACGCTATGGACCTCCAGGTTTTTTGGATCAACAT |
| | | CAGCCATTGGCTTTGCTCTTGTTCGATATATTCGCTGGTGGGAGGCCTTTTCTTGGTTG |
| | | AACTGCCCGCTTGTGATATGGTCCGTGGCCATGTTCTTTCTGTTTGAAACACTCTATGG |
| | | AGCCCTATTCTATTCTGTCAAGCGACAAGAAGGGAGATCCCAGCGTGGAATCAAGCAC |
| | | AAAGAGAGGTAGACAAATCGCTCTTAAATATATACCTAAAGAACATTAAAGCTATATTAT |
| | | AAGCAAAGATACGTAAATTTTGCTTATATTATTATACACATATCATATTTCTATATTTTTAA |
| | | GATTTGGTTATATAATGTACGTAATGCAAAGGAAATAAATTTTATACATTATTGAACAGC |
| | | GTCCAAGTAACTACATTATGTGCACTAATAGTTTAGCGTCGTGAAGACTTTATTGTGTCG |
| | | CGAAAAGTAAAAATTTTAAAAATTAGAGCACCTTGAACTTGCGAAAAAGGTTCTCATCAA |
| | | CTGTTTAAAAGGAGGATATCAGGTCCTATTTCTGACAAACAATATACAAATTTAGTTTCA |
| | | AAGATGAATCAGTGCGCGAAGGACATAACTCAACAGTTTATTCCTGGCATCCACTAAAT |
| | | ATAATGGAGCCCGCTTTTTAAGCTGGCATCCAGAAAAAAAAAGAATCCCAGCACCAAAA |
| | | TATTGTTTTCTTCACCAACCATCAGTTCATAGGTCCATTCTCTTAGCGCAACTACAGAGA |
| | | ACAGGGGCACAAACAGGCAAAAAACGGGCACAACCTCAATGGAGTGATGCAACCTGC |
| | | CTGGAGTAAATGATGACACAAGGCAATTGACCCACGCATGTATCTATCTCATTTTCTTA |
| | | CACCTTCTATTACCTTCTGCTCTCTCTGATTTGGAAAAAGCTGAAAAAAAAGGTTGAAAC |
| | | CAGTTCCCTGAAATTATTCCCCTACTTGACTAATAAGTATATAAAGACGGTAGGTATTGA |
| | | TTGTAATTCTGTAAATCTATTTCTTAAACTTCTTAAATTCTACTTTTATAGTTAGTCTTTTT |
| | | TTTAGTTTTAAAACACCAAGAACTTAGTTTCGAATAAACACACATAAACAAACAAAATGG |
| | | CGGCACTTCCGGACGTTGCCTCCATTCCCATCCCTCTGGTGGCAACCCTAGGCATTGC |
| | | CCCTCTAATTTTCTATCTCGTCCTTGATAGAATTAGCCCCTTGTGGCCAAATTCCAAAGC |
| | | TTTCCTGATTGGCAAGAAGAAACCGGAGACCGTGACATCGTTCGAGTGCCCATATGCC |
| | | TACATCCGTCAGATCTATGGGAAGTATCACTGGGAGCCATTCGTACAGAAGCTGTCTC |
| | | CGAGGCTTAAGGATGAGGATCCGGCCAAATATAAGATGGTTCTGGAGATAATGGATGC |
| | | AATCCACCTGTGTCTGATGCTAGTTGACGATATAACTGACAATAGCGACTATCGAAAAG |
| | | GCAAGCCAGCAGCCCACCGGATATATGGCCCTTCAGAGACAGCAAATCGCGCTTACTA |
| | | CCGAGTCACCCAGATTCTAAACAAGACCGTGCAAAAGTTCCCCAAGCTGGCCAAGTTC |
| | | CTGCTTCAGAATCTGGAAGAAATTCTCGAAGGCCAAGACCTGTCACTAATCTGGCGAC |
| | | GGGATGGACTGGGTAGCCTTTCGACTGTTCCTGATGAGCGAGTTGCAGCCTATCGCAA |
| | | GATGGCGTCATTGAAAACTGGGGCGTTATTCCGGCTGCTGGGGCAATTGGTGATGGA |
| | | GGACCAATCGATGGACGGGACGATGACTACTCTTGCGTGGTGCTCTCAGCTGCAGAAT |
| | | GACTGCAAGAATGTCTACTCATCTGAATATGCTAAGGCCAAAGGGGCGCTTGCCGAAG |
| | | ACCTCCGAAATCGAGAGCTCTCATTTCCAATTATCCTCGCGCTGGAAGCTCCTGAAGG |
| | | GCATTGGGTCGCCAGTGCTTTGGAGACCAGCTCACCGCGCAACATTCGCAAGGCGCT |
| | | TGCTGTGATTCAGAGTGAGAGAGTGCGCAATGCTTGTTTCAAGGAGCTCAAGTCGGCG |
| | | AGTGCTTCGGTCCAGGACTGGTTGGCTATTTGGGGACGGAACGAGAAAATGAACTTGA |
| | | AGAGCCAGCAGACGTAGAGTGCTTTTAACTAAGAATTATTAGTCTTTTCTGCTTATTTTT |
| | | TCATCATAGTTTAGAACACTTTATATTAACGAATAGTTTATGAATCTATTTAGGTTTAAAA |
| | | ATTGATACAGTTTTATAAGTTACTTTTTCAAAGACTCGTGCTGTCTATTGCATAATGCACT |
| | | GGAAGGGGAAAAAAAAGGTGCACACGCGTGGCTTTTTCTTGAATTTGCAGTTTGAAAAA |
| | | TAACTACATGGATGATAAGAAAACATGGAGTACAGTCACTTTGAGAACCTTCAATCAGC |
| | | TGGTAACGTCTTCGTTAATTGGATACTCAAAAAAGATGGATAGCATGAATCACAAGATG |
| | | GAAGGAAATGCGGGCCACGACCACAGTGATATGCATATGGGAGATGGAGATGATACCT |
| | | TATATCTAGGAACCCATCAGGTTGGTGGAAGATTACCCGTTCTAAGACTTTTCAGCTTC |
| | | CTCTATTGATGTTACACCTGGACACCCCTTTTCTGGCATCCAGTTTTTAATCTTCAGTGG |
| | | CATGTGAGATTCTCCGAAATTAATTAAAGCAATCACATTCTCTCGGATACCACCTC |
| | | GGTTGAAACTGACAGGTGGTFTGTTACGCATGCTAATGCAAAGGAGCCTATATACCTTT |
| | | GGCTCGGCTGCTGTAACAGGGAATATAAAGGGCAGCATAATTTAGGAGTTTAGTGAAC |
| | | TTGCAACATTTACTATTTTCCCTTCTTACGTAAATATTTTTCTTTTTAATTCTAAATCAATC |
| | | TTTTTCAATTTTTTGTTTGTATTCTTTTCTTGCTTAAATCTATAACTACAAAAAACACATAC |
| | | ATAAACTAAAAATGGCCAATGCCCAGCAACCCCCCTTTCGCATCCTTATTGTGGGCGGT |
| | | TCTGTCGCAGGCCTCATCCTTGCGCACTGTCTCGAACGCGCCAATATAGAGTACCTCA |
| | | TACTCGAAAAAGGAGAAGATGTTGCTCCACAAGTTGGTGCCTCGATAGGTATCATGCC |
| | | AAATGGCGGACGGATCCTCGAGCAACTGGGCCTATTTGGGGAGATTGAGCGTGTGAT |
| | | CGAGCCGTTGCATCAGGCGAATATCAGCTATCCAGATGGGTTCTGCTTTAGTAACGTCT |
| | | ATCCTAAGGTTCTTGGCGACAGGTTCGGATACCCGGTTGCATTCTTGGACCGGCAGAA |
| | | GTTCCTGCAGATTGCATATGAGGGGCTGAGAAAGAAGCAGAATGTTCTCACCGGTAAA |
| | | AGGGTAGTTGGACTGCGACAGTCGGATCAAGGGACTGCTGTTTCTGTGGCTGACGGG |
| | | ACAGAGTATGAGGCGGATCTCGTGGTTGGTGCTGATGGAGTACATAGTCGGGTGAGAA |
| | | GTGAGATTTGGAAGATGGCGGAAGAGAATCAGCCTGCATCAGTTTCGACACGTGAAAG |
| | | AAGAAGCATGACTGTTGAATATGTCTGCGTTTTCGGGATTTCATCAGCCATCCCAGGGC |
| | | TCGAGATAAGCGAACAGATCAACGGTATTTTCGACCATCTATCCATTCTAACAATCCAT |
| | | GGCAGACATGGTCGCGTGTTCTGGTTCGTGATCCAGAAGCTGGATAGGAAGTACGTCT |
| | | ATCCTGATGTCCCGCGATTCTCAGACGAGGATGCCGTACAGCTCTTCGATCGGGTCAA |
| | | ACACGTGCGGTTCTGGAAAAACATCTGTGTGGGGGACTTGTGGAAGAACAGAGAGGT |
| | | GTCCTCGATGACAGCGCTGGAGGAGGGAGTGTTCGAGACATGGCATCATGATAGGAT |
| | | GGTTTTGATTGGAGATAGCGTTCACAAGATGACGCCCAACTTTGGCCAAGGAGCTAATT |
| | | CAGCCATCGAGGATGCTGCCGCGCTCTCTTCCCTTCTACATGATCTCGTCAACGCCCG |
| | | TGGAGTTTGCAAGCCATCGAATGTCCAGATTCAGCATCTCCTCAAGCAGTATCGGGAG |
| | | ACCCGATACACTCGCATGGTAGGCATGTGTCGCACCGCGGCTTCAGTCTCTCGGATTC |
| | | AGGCCCGAGATGGCATCCTCAACACCGTCTTTGGACGATATTGGGCACCTTATGCTGG |
| | | CAACCTGCCTGCTGACCTGGCATCAAAAGTGATGGCAGATGCAGAGGTTGTTACTTTT |
| | | CTGCCCTTGCCAGGGCGCTCAGGACCGGGCTGGGAGATGTACAGACGAAAGGGGAA |
| | | GGGAGGGCAGGTGCAATGGGTGCTTATAATCTTAAGCTTACTTACGATTGGTGGATTG |
| | | TGCATCTGGCTACAAAGCAATGCGTTGAGTAGATAAGGAGATTGATAAGACTTTTCTAG |
| | | TTGCATATCTTTTATATTTAAATCTTATCTATTAGTTAATTTTTTGTAATTTATCCTTATATA |
| | | TAGTCTGGTTATTCTAAAATATCATTTCAGTATCTAAAAATTCCCCTCTTTTTTCAGTTAT |
| | | ATCTTAACAGGCGACAGTCCAAATGTTGATTTATCCCAGTCCGATTCATCAGGGTTGTG |
| | | AAGCATTTTGTCAATGGTCGAAATCACATCAGTAATAGTGCCTCTTACTTGCCTCATAGA |
| | | ATTTCTTTCTCTTAACGTCACCGTTTGGTCTTTTATAGTTTCGAAATCTATGGTGATACCA |
| | | AATGGTGTTCCCAATTCATCGTTACGGGCGTATTTTTTACCAATTGAAGTATTGGAATCG |
| | | TCAATTTTAAAGTATATCTCTCTTTTACGTAAAGCCTGCGAGATCCTCTTAAGTATAGCG |
| | | GGGAAGCCATCGTTATTCGATATTGTCGTAACAAATACTTTGATCGGCGCTATGCGGCC |
| | | GCCACCGCGGTGGAGCTCCAGCTTTTGTTCCCTTTAGTGAGGGTTAATTGCGCGCTTG |
| | | GCGTAATCATGGTCATAGCTGTTTCCTGTGTGAAATTGTTATCCGCTCACAATTCCACA |
| | | CAACATAGGAGCCGGAAGCATAAAGTGTAAAGCCTGGGGTGCCTAATGAGTGAGGTAA |
| | | CTCACATTAATTGCGTTGCGCTCACTGCCCGCTTTCCAGTCGGGAAACCTGTCGTGCC |
| | | AGCTGCATTAATGAATCGGCCAACGCGCGGGGAGAGGCGGTTTGCGTATTGGGCGCT |
| | | CTTCCGCTTCCTCGCTCACTGACTCGCTGCGCTCGGTCGTTCGGCTGCGGCGAGCGG |
| | | TATCAGCTCACTCAAAGGCGGTAATACGGTTATCCACAGAATCAGGGGATAACGCAGG |
| | | AAAGAACATGTGAGCAAAAGGCCAGCAAAAGGCCAGGAACCGTAAAAAGGCCGCGTT |
| | | GCTGGCGTTTTTCCATAGGCTCCGCCCCCCTGACGAGCATCACAAAAATCGACGCTCA |
| | | AGTCAGAGGTGGCGAAACCCGACAGGACTATAAAGATACCAGGCGTTTCCCCCTGGAA |
| | | GCTCCCTCGTGCGCTCTCCTGTTCCGACCCTGCCGCTTACCGGATACCTGTCCGCCTT |
| | | TCTCCCTTCGGGAAGCGTGGCGCTTTCTCATAGCTCACGCTGTAGGTATCTCAGTTCG |
| | | GTGTAGGTCGTTCGCTCCAAGCTGGGCTGTGTGCACGAACCCCCCGTTCAGCCCGAC |
| | | CGCTGCGCCTTATCCGGTAACTATCGTCTTGAGTCCAACCCGGTAAGACACGACTTAT |
| | | CGCCACTGGCAGCAGCCACTGGTAACAGGATTAGCAGAGCGAGGTATGTAGGCGGTG |
| | | CTACAGAGTTCTTGAAGTGGTGGCCTAACTACGGCTACACTAGAAGGACAGTATTTGGT |
| | | ATCTGCGCTCTGCTGAAGCCAGTTACCTTCGGAAAAAGAGTTGGTAGCTCTTGATCCG |
| | | GCAAACAAACCACCGCTGGTAGCGGTGGTTTTTTTGTTTGCAAGCAGCAGATTACGCG |
| | | CAGAAAAAAAGGATCTCAAGAAGATCCTTTGATCTTTTCTACGGGGTCTGACGCTCAGT |
| | | GGAACGAAAACTCACGTTAAGGGATTTTGGTCATGAGATTATCAAAAAGGATCTTCACC |
| | | TAGATCCTTTTAAATTAAAAATGAAGTTTTAAATCAATCTAAAGTATATATGAGTAAACTT |
| | | GGTCTGACAGTTACCAATGCTTAATCAGTGAGGCACCTATCTCAGCGATCTGTCTATTT |
| | | CGTTCATCCATAGTTGCCTGACTCCCCGTCGTGTAGATAACTACGATACGGGAGGGCT |
| | | TACCATCTGGCCCCAGTGCTGCAATGATACCGCGAGACCCACGCTCACCGGCTCCAG |
| | | ATTTATCAGCAATAAACCAGCCAGCCGGAAGGGCCGAGCGCAGAAGTGGTCCTGCAA |
| | | CTTTATCCGCCTCCATCCAGTCTATTAATTGTTGCCGGGAAGCTAGAGTAAGTAGTTCG |
| | | CCAGTTAATAGTTTGCGCAACGTTGTTGCCATTGCTACAGGCATCGTGGTGTCACGCTC |
| | | GTCGTTTGGTATGGCTTCATTCAGCTCCGGTTCCCAACGATCAAGGCGAGTTACATGAT |
| | | CCCCCATGTTGTGCAAAAAAGCGGTTAGCTCCTTCGGTCCTCCGATCGTTGTCAGAAG |
| | | TAAGTTGGCCGCAGTGTTATCACTCATGGTTATGGCAGCACTGCATAATTCTCTTACTG |
| | | TCATGCCATCCGTAAGATGCTTTTCTGTGACTGGTGAGTACTCAACCAAGTCATTCTGA |
| | | GAATAGTGTATGCGGCGACCGAGTTGCTCTTGCCCGGCGTCAATACGGGATAATACCG |
| | | CGCCACATAGCAGAACTTTAAAAGTGCTCATCATTGGAAAACGTTCTTCGGGGCGAAAA |
| | | CTCTCAAGGATCTTACCGCTGTTGAGATCCAGTTCGATGTAACCCACTCGTGCACCCAA |
| | | CTGATCTTCAGCATCTTTTACTTTCACCAGCGTTTCTGGGTGAGCAAAAACAGGAAGGC |
| | | AAAATGCCGCAAAAAAGGGAATAAGGGCGACACGGAAATGTTGAATACTCATACTCTTC |
| | | CTTTTTCAATATTATTGAAGCATTTATCAGGGTTATTGTCTCATGAGCGGATACATATTT |
| | | GAATGTATTTAGAAAAATAAACAAATAGGGGTTCCGCGCACATTTCCCCGAAAAGTGCC |
| | | ACCTGAACGAAGCATCTGTGCTTCATTTTGTAGAACAAAAATGCAACGCGAGAGCGCTA |
| | | ATTTTTCAAACAAAGAATCTGAGCTGCATTTTTACAGAACAGAAATGCAACGCGAAAGC |
| | | GCTATTTTACCAACGAAGAATCTGTGCTTCATTTTTGTAAAACAAAAATGCAACGCGAGA |
| | | GCGCTAATTTTTCAAACAAAGAATCTGAGCTGCATTTTTACAGAACAGAAATGCAACGC |
| | | GAGAGCGCTATTTTACCAACAAAGAATCTATACTTCTTTTTTGTTCTACAAAAATGCATC |
| | | CCGAGAGCGCTATTTTTCTAACAAAGCATCTTAGATTACTTTTTTTCTCCTTTGTGCGCT |
| | | CTATAATGCAGTCTCTTGATAACTTTTTGCACTGTAGGTCCGTTAAGGTTAGAAGAAGG |
| | | CTACTTTGGTGTCTATTTTCTCTTCCATAAAAAAAGCCTGACTCCACTTCCCGCGTTTAC |
| | | TGATTACTAGCGAAGCTGCGGGTGCATTTTTTCAAGATAAAGGCATCCCCGATTATATT |
| | | CTATACCGATGTGGATTGCGCATACTTTGTGAACAGAAAGTGATAGCGTTGATGATTCT |
| | | TCATTGGTCAGAAAATTATGAACGGTTTCTTCTATTTTGTCTCTATATACTACGTATAGG |
| | | AAATGTTTACATTTTCGTATTGTTTTCGATTCACTCTATGAATAGTTCTTACTACAATTTTT |
| | | TTGTCTAAAGAGTAATACTAGAGATAAACATAAAAAATGTAGAGGTCGAGTTTAGATGC |
| | | AAGTTCAAGGAGCGAAAGGTGGATGGGTAGGTTATATAGGGATATAGCACAGAGATAT |
| | | ATAGCAAAGAGATACTTTTGAGCAATGTTTGTGGAAGCGGTATTCGCAATATTTTAGTA |
| | | GCTCGTTACAGTCCGGTGCGTTTTTGGTTTTTTGAAAGTGCGTCTTCAGAGCGCTTTTG |
| | | GTTTTCAAAAGCGCTCTGAAGTTCCTATACTTTCTAGAGAATAGGAACTTCGGAATAGG |
| | | AACTTCAAAGCGTTTCCGAAAACGAGCGCTTCCGAAAATGCAACGCGAGCTGCGCACA |
| | | TACAGCTCACTGTTCACGTCGCACCTATATCTGCGTGTTGCCTGTATATATATATACATG |
| | | AGAAGAACGGCATAGTGCGTGTTTATGCTTAAATGCGTACTTATATGCGTCTATTTATGT |
| | | AGGATGAAAGGTAGTCTTAGTACCTCCTGTGATATTATCCCATTCCATGCGGGGTATCGT |
| | | ATGCTTCCTTCAGCACTACCCTTTAGCTGTTCTATATGCTGCCACTCCTCAATTGGATTA |
| | | GTCTCATCCTTCAATGCTATCATTTCCTTTGATATTGGATCATACTAAGAAACCATTATTA |
| | | TCATGACATTAACCTATAAAAATAGGCGTATCACGAGGCCCTTTCGTCTCGCGCGTTTC |
| | | GGTGATGACGGTGAAAACCTCTGACACATGCAGCTCCCGGAGACGGTCACAGCTTGTC |
| | | TGTAAGCGGATGCCGGGAGCAGACAAGCCCGTCAGGGCGCGTCAGCGGGTGTTGGC |
| | | GGGTGTCGGGGCTGGCTTAACTATGCGGCATCAGAGCAGATTGTACTGAGAGTGCAC |
| | | CATATCGACTACGTCGTAAGGCCGTTTCTGACAGAGTAAAATTCTTGAGGGAACTTTCA |
| | | CCATTATGGGAAATGCTTCAAGAAGGTATTGACTTAAACTCCATCAAATGGTCAGGTCA |
| | | TTGAGTGTTTTTTATTTGTTGTATTTTTTTTTTTTTAGAGAAAATCCTCCAATATCAAATTA |
| | | GGAATCGTAGTTTCATGATTTTCTGTTACACCTAACTTTTTGTGTGGTGCCCTCCTCCTT |
| | | GTCAATATTAATGTTAAAGTGCAATTCTTTTTCCTTATCACGTTGAGCCATTAGTATCAAT |
| | | TTGCTTACCTGTATTCCTTTACTATCCTCCTTTTTCTCCTTCTTGATAAATGTATGTAGAT |
| | | TGCGTATATAGTTTCGTCTACCCTATGAACATATTCCATTTTGTAATTTCGTGTCGTTTCT |
| | | ATTATGAATTTCATTTATAAAGTTTATGTACAAATATCATAAAAAAAGAGAATCTTTTTAA |
| | | GCAAGGATTTTCTTAACTTCTTCGGCGACAGCATCACCGACTTCGGTGGTACTGTTGGA |
| | | ACCACCTAAATCACCAGTTCTGATACCTGCATCCAAAACCTTTTTAACTGCATCTTCAAT |
| | | GGCCTTACCTTCTTCAGGCAAGTTCAATGACAATTTCAACATCATTGCAGCAGACAAGA |
| | | TAGTGGCGATAGGGTCAACCTTATTCTTTGGCAAATCTGGAGCAGAACCGTGGCATGG |
| | | TTCGTACAAACCAAATGCGGTGTTCTTGTCTGGCAAAGAGGCCAAGGACGCAGATGGC |
| | | AACAAACCCAAGGAACCTGGGATAACGGAGGCTTCATCGGAGATGATATCACCAAACA |
| | | TGTTGCTGGTGATTATAATACCATTTAGGTGGGTTGGGTTCTTAACTAGGATCATGGCG |
| | | GCAGAATCAATCAATTGATGTTGAACCTTCAATGTAGGGAATTCGTTCTTGATGGTTTCC |
| | | TCCACAGTTTTTCTCCATAATCTTGAAGAGGCCAAAAGATTAGCTTTATCCAAGGACCAA |
| | | ATAGGCAATGGTGGCTCATGTTGTAGGGCCATGAAAGCGGCCATTCTTGTGATTCTTTG |
| | | CACTTCTGGAACGGTGTATTGTTCACTATCCCAAGCGACACCATCACCATCGTCTTCCT |
| | | TTCTCTTACCAAAGTAAATACCTCCCACTAATTCTCTGACAACAACGAAGTCAGTACCTT |
| | | TAGCAAATTGTGGCTTGATTGGAGATAAGTCTAAAAGAGAGTCGGATGCAAAGTTACAT |
| | | GGTCTTAAGTTGGCGTACAATTGAAGTTCTTTACGGATTTTTAGTAAACCTTGTTCAGGT |
| | | CTAACACTACCGGTACCCCATTTAGGACCAGCCACAGCACCTAACAAAACGGCATCAA |
| | | CCTTCTTGGAGGCTTCCAGCGCCTCATCTGGAAGTGGGACACCTGTAGCATCGATAGC |
| | | AGCACCACCAATTAAATGATTTTCGAAATCGAACTTGACATTGGAACGAACATCAGAAA |
| | | TAGCTTTAAGAACCTTAATGGCTTCGGCTGTGATTTCTTGACCAACGTGGTCACCTGGC |
| | | AAAACGACGATCTTCTTAGGGGCAGACATAGGGGCAGACATTAGAATGGTATATCCTT |
| | | GAAATATATATATATATTGCTGAAATGTAAAAGGTAAGAAAAGTTAGAAAGTAAGACGAT |
| | | TGCTAACCACCTATTGGAAAAAACAATAGGTCCTTAAATAATATTGTCAACTTCAAGTAT |
| | | TGTGATGCAAGCATTTAGTCATGAACGCTTCTTCTATTCTTATATGAAAAGCCGGTTCCGG |
| | | CCTCTCACCTTTCCTTTTTCTCCCAATTTTTCAGTTGAAAAAGGTATATGCGTCAGGCGA |
| | | CCTCTGAAATTAACAAAAAATTTCCAGTCATCGAATTTGATTCTGTGCGATAGCGCCCCT |
| | | GTGTGTTCTCGTTATGTTGAGGAAAAAAATAATGGTTGCTAAGAGATTCGAACTCTTGC |
| | | ATCTTACGATACCTGAGTATTCCCACAGTTAACTGCGGTCAAGATATTTCTTGAATCAG |
| | | GCGCCTTAGACCGCTCGGCCAAACAACCAATTACTTGTTGAGAAATAGAGTATAATTAT |
| | | CCTATAAATATAACGTTTTTGAACACACATGAACAAGGAAGTACAGGACAATTGATTTTG |
| | | AAGAGAATGTGGATTTTGATGTAATTGTTGGGATTCCATTTTTAATAAGGCAATAATATT |
| | | AGGTATGTGGATATACTAGAAGTTCTCCTCGACCGTCGATATGCGGTGTGAAATACCG |
| | | CACAGATGCGTAAGGAGAAAATACCGCATCAGGAAATTGTAAACGTTAATATTTTGTTA |
| | | AAATTCGCGTTAAATTTTTGTTAAATCAGCTCATTTTTTAACCAATAGGCCGAAATCGGC |
| | | AAAATCCCTTATAAATCAAAAGAATAGACCGAGATAGGGTTGAGTGTTGTTCCAGTTTG |
| | | GAACAAGAGTCCACTATTAAAGAACGTGGACTCCAACGTCAAAGGGCGAAAAACCGTC |
| | | TATCAGGGCGATGGCCCACTACGTGAACCATCACCCTAATCAAGTTTTTTGGGGTCGA |
| | | GGTGCCGTAAAGCACTAAATCGGAACCCTAAAGGGAGCCCCCGATTTAGAGCTTGACG |
| | | GGGAAAGCCGGCGAACGTGGCGAGAAAGGAAGGGAAGAAAGCGAAAGGAGCGGGCG |
| | | CTAGGGCGCTGGCAAGTGTAGCGGTCACGCTGCGCGTAACCACCACACCCGCCGCGC |
| | | TTAATGCGCCGCTACAGGGCGCGTCGCGCCATTCGCCATTCAGGCTGCGCAACTGTT |
| | | GGGAAGGGCGATCGGTGCGGGCCTCTTCGCTATTACGCCAGCTGGCGAAAGGGGGAT |
| | | GTGCTGCAAGGCGATTAAGTTGGGTAACGCCAGGGTTTTCCCAGTCACGACGTTGTAA |
| | | AACGACGGCCAGTGAGCGCGCGTAATACGACTCACTATAGGGCGAATTGGGTACCGG |
| | | GCCCCCCCTCGAGGTCGACGGTATCGATAAGCTTGATATCGAATTCCTGCAGCCCGGG |
| | | GGATCCACTAGTTCTAGATTAATTAA |

Doctrine of Equivalents

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A DNA molecule composition comprising:
at least one exogenous DNA vector comprising at least two different production-phase promoters;
wherein the two production-phase promoters are each capable of repressing heterologous expression of an exogenous gene in a Saccharomyces cerevisiae cell when the S. cerevisiae cell predominantly exhibits anaerobic energy metabolism; and
wherein the two production-phase promoters are each also capable of inducing heterologous expression of the exogenous gene in the S. cerevisiae cell when the S. cerevisiae cell predominantly exhibits aerobic energy metabolism.
